(19) United States
(12) Patent Application Publication
(10) Pub. No.: US 2026/0079455 A1
(43) Pub. Date: Mar. 19, 2026

(54) COMPUTER-IMPLEMENTED SYSTEM AND METHOD FOR DETERMINING AN INTERIM LIFE SAFETY MEASURE (ILSM) TO AUTOMATICALLY CONTROL ONE OR MORE ASSETS WITHIN ONE OR MORE SMOKE COMPARTMENTS IN A FACILITY

(71) Applicant: Healthcare Facility Compliance Corp, Woodcliff Lake, NJ (US)

(72) Inventors: Ilan Nachmany, Upper Saddle River, NJ (US); Sumer Mathur, Great Falls, VA (US)

(21) Appl. No.: 19/377,263

(22) Filed: Nov. 3, 2025

Related U.S. Application Data
(63) Continuation-in-part of application No. 18/180,890, filed on Mar. 9, 2023, now abandoned.

(51) Int. Cl. G05B 13/02

(52) U.S. Cl. CPC G05B 13/0265

(57) ABSTRACT

A computer-implemented system and method for determining an ILSM to automatically control assets within smoke compartments in facility is disclosed. The computer-implemented system analyzes potential risk factors associated with individuals in smoke compartments of facility, based on type of smoke compartments. Further, the computer-implemented system classifies assets associated with smoke compartments into risk levels, based on asset classes and location environment associated assets comprising risk levels. Additionally, the computer-implemented system assigns risk assessment scores to inspection points corresponding to classified assets. Furthermore, the computer-implemented system determines ILSMs. Additionally, the computer-implemented system configures information associated with the selected ILSM actions with IoT controllers for adapting the IoT controllers to automatically control the assets within smoke compartments to protect the individuals in the smoke compartments of the facility. The computer-implemented system further provides controlled activities of the assets, to users through user interfaces associated with electronic devices of the users.

### CROSS REFERENCE TO RELATED APPLICATIONS

This application is a continuation-in-part of U.S. patent application Ser. No. 18/180,890, filed on Mar. 9, 2023, entitled “SYSTEM AND METHOD FOR DETERMINING AN INTERIM LIFE SAFETY MEASURE (ILSM) FOR ONE OR MORE SMOKE COMPARTMENTS IN A FACILITY” which claims the benefit of U.S. patent application having Ser. No. 16/190,908, filed on Nov. 14, 2018, entitled “Enterprise Mobile, Cloud Based Health Care Risk Assessment System,” which claims the benefit of continuation-in-part of U.S. patent application having Ser. No. 16/184,891, filed on Nov. 8, 2018, and entitled “Enterprise Mobile, Cloud Based Health Care Compliance System,” which again claims the priority of U.S. provisional patent application having Ser. No. 62/583,453, filed on Nov. 8, 2017, all of which are hereby incorporated by reference in their entirety.

### TECHNICAL FIELD

Embodiments of present disclosure generally relate to a risk assessment of a facility for a health care compliance and more particularly embodiments of present disclosure relate to a computer-implemented system and method for determining an interim life safety measure (ILSM) to automatically control one or more assets within one or more smoke compartments in the facility.

### BACKGROUND

Generally, to improve healthcare quality, organizations have been developed to inspect and accredit health care facilities. Examples of such organizations include, but are not limited to, centers for Medicare and medic-aid services (CMS) or an accredited independent, not-for-profit organizations, i.e., the joint commission (TJC), det norske veritas (DNV), a center for improvement in healthcare quality (CIHQ), a healthcare facilities accreditation program (HFAP), and the like. For example, the TJC helps in continuously improving health care for the public, by evaluating health care organizations. Further, the TJC accredits and certifies more than 21,000 health care organizations and programs in the United States, including hospitals and health care organizations that provide ambulatory and office-based surgery, behavioral health, home health care, laboratory, and nursing care center services. This organization dominates the market regulating 15,716 healthcare facilities in the United States, 3959 Hospitals, 848 Nursing care Center 363 Critical Access Hospitals 1941 Ambulatory Care 3017 Behavioral Health 5298 Home Care and 290 Office Based Surgery, a total of 78% of the US healthcare facilities.

Further, hospitals may depend to varying degrees on receipt of Federal funds to meet their increasing budget needs. Along with partial Federal financing of hospitals, specific Federal requirements have been introduced requiring hospitals to demonstrate compliance to receive Medicare/Medicaid funding. To demonstrate compliance, hospitals were initially given two options by Congress: Either submit to a federal audit or achieve accreditation from The Joint Commission (originally known as TJC on accreditation of hospitals, and later as the joint commission on accreditation of healthcare organizations). Passing an audit by either of these two organizations would give a hospital “Deemed Status” for a 3-year period which would enable them to collect Medicare/Medicaid funds while they hold this status. Not obtaining or loosing this status would prohibit them from collecting this funding while they were not in “Deemed Status.”

Over the years the healthcare environment has gotten far more complex from a regulatory and technology standpoint as well as a medical and business standpoint. As this has occurred, the number of regulations has increased dramatically; the regulations themselves have become far more complex; documentation requirements have greatly increased; yet due to cost constraints, spending on programs and staffing has been continually cut by the healthcare providers. Currently, hospitals and healthcare facilities lack the ability to conduct risk analysis at the time of the deficiency's discovery, putting pressure on management to conduct risk analysis within a specific mandatory time frame, which may be missed. In addition, incorporating and monitoring multiple interim life safety measures (ILSM) requirements, which are health and safety measures that were put in place to protect the safety of patients, visitors, and staff who work in hospitals and other healthcare facilities, may not happen on time. Since construction, inspection and maintenance activities can impact the facility's life safety; regulations were put in place, which dictate specific mandatory actions, based on risk level, and must be performed with a specific pre-defined timeframe. Facilities must also develop their own ILSM policy for the protection, safety, and health of patients, in order to alleviate hazards, incorporating measures of their own, together with the regulated mandatory requirements.

When under construction or when a failed inspection accrues, a list of Interim Life Safety Measures needs to be determined. The reason for determining and activating the specific ILSM is to protect the safety of patients, visitors, and those who work in the hospital. They are based on conducting risk analysis, to determine which of the ILSM items must be activated and they need to occur within a certain time, from the time of discovery. It is almost impossible for healthcare facility departments to be compliant, at all times, and meet all ILSM compliant requirements, specifically for smoke compartments, which is a type of a passive fire protection within a building and an area within a fire compartment that is required to be separated by barriers (on all sides) such as walls, doors, and/or floors and ceilings having the appropriate resistance to the spread of smoke. In addition to this, the facilities must require an automated system that needs to automatically control assets in the smoke compartments, in order to protect individuals in the smoke compartments of the facilities.

Hence, there is a need to address at least the aforementioned issues/problems in the existing approaches by providing an improved computer-implemented system and method for determining an interim life safety measure (ILSM) to automatically control one or more assets in one or more smoke compartments in the facility, to ensure the ILSM requirements for a safe environment for all a hospital's occupants are completed, while ensuring and validating that the required activities are performed on time. In addition, the computer-implemented system should ensure a complete documentation audit trail is maintained and all responsible parties are notified, thus providing hospitals with the ability to assure ongoing compliance and reduce cost by replacing their current existing costly manual inspections.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

An aspect of the present disclosure includes a computer-implemented system for determining an interim life safety measure (ILSM) to automatically control one or more assets within one or more smoke compartments in a facility. The computer-implemented system includes one or more hardware processors and a memory. The memory is connected to the one or more hardware processors. The memory comprises a set of program instructions in form of a plurality of subsystems that are configured to be executed by the one or more hardware processors. The plurality of subsystems comprises a potential risk analyzing subsystem configured to: (a) receive real-time sensor data comprising at least one of: smoke density, temperature, humidity, environmental factors and image data captured from the one or more smoke compartments of the facility; and (b) analyze a plurality of potential risk factors associated with one or more individuals in the one or more smoke compartments of the facility, based on a type of the one or more smoke compartments. The potential risk factors may be a potential harm/hazards to the one or more individuals. The individuals may include, but are not limited to, health care workers, workers, labors, patients, staff, health care providers, vendors, technicians, maintenance staff, visitors, and the like. The type of the one or more smoke compartments may include, but are not limited to, smoke dampers, fire-rated walls, and fire doors smoke barriers, smoke-tight partitions, and the like.

For analyzing the plurality of potential risk factors, the potential risk analyzing subsystem is configured to utilize one or more trained Artificial Intelligence (AI)-based machine learning models, by at least one of: (a) detecting at least one of: smoke and flames in the one or more smoke compartments, based on large datasets of smoke and non-smoke image data; (b) analyzing the real-time sensor data from at least one of: temperature and humidity sensors, to predict fire risks in the one or more smoke compartments; (c) modeling causal relationships between one or more fire risk factors contributing to the fire risks in the one or more smoke compartments; (d) classifying sensor data patterns comprising variations in temperature, humidity, and smoke density, indicative of the fire risks in the one or more smoke compartments; and (e) providing recommendations for at least one of: preventive measures and emergency response based on sensor data patterns associated with potential fire risks. The one or more trained AI-based machine learning models comprise at least one of: first convolutional neural networks (CNNs), recurrent neural networks (RNNs), Bayesian networks, first support vector machines (SVMs), and decision trees.

The plurality of subsystems further comprises a risk level determining subsystem configured to determine a plurality of risk levels for each of the one or more smoke compartments, based on the analyzed plurality of potential risk factors. The plurality of subsystems further comprises an asset classifying subsystem, in response to determining the plurality of risk levels, configured to classify one or more assets associated with each of the one or more smoke compartments into the plurality of risk levels, based on at least one of: one or more asset classes and a location environment associated with each of the one or more assets in each of the one or more smoke compartments comprising the plurality of risk levels. The one or more asset classes are generated using at least one of: asset class inheritance-based AI models and asset class acquisition-based AI models. At least one of: the asset class inheritance-based AI models and asset class acquisition-based AI models is trained to: (a) analyze image data and historical data associated with the one or more assets; and (b) classify the one or more assets into asset classes based on the analyzed image data and historical data. At least one of the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, and Generative Adversarial Networks (GANs).

The plurality of subsystems further comprises a score assigning subsystem configured to assign a plurality of risk assessment scores to each of one or more inspection points corresponding to the classified one or more assets. The risk assessment score corresponds to at least one of an importance and a potential harm created when the one or more inspection points fail an inspection. Each of the one or more inspection points corresponds to a requirement of an element of performance (EP). For example, mandated standards include a plurality of elements of performance (EPs) to indicate what needs to be completed to satisfy the standard. An example EP could be “the fire doors need to be inspected once per year.” Embodiments of the present disclosure look not only at what frequency an asset should be inspected, but also how the inspection should be performed (what should be the inspection points). Each inspection point indicates a requirement of the respective EP. For example, an inspection point can be an asset inspection, a document, or an action to be taken. More specifically, an inspection point is any requirement relating to: assets, machinery, instruments and other facility optional equipment, facility condition inspections, construction status and/or inspections relating to having valid current supporting document, as they relate to environmental care, life safety, and emergency management.

The plurality of subsystems further comprises an aggregated score determining subsystem configured to determine an aggregated score of the plurality of risk assessment scores associated with one or more failed inspection points for the one or more assets within each of the one or more smoke compartments. The plurality of subsystems further comprises an action storing subsystem configured to store information associated with a plurality of Interim Life Safety Measure (ILSM) actions and a plurality of correlating deficiency assets, in a database. Each ILSM action defines an action related to protecting the one or more individuals at the facility. Each correlating deficiency asset defines a correlation between a deficiency encountered at the facility and at least one ILSM action.

The plurality of subsystems further comprises an Interim Life Safety Measure (ILSM) determining subsystem configured to determine a plurality of ILSMs for each of the one or more assets within each of the one or more smoke compartments, when the aggregated score is greater than a pre-determined threshold value for each of the one or more assets within each of the one or more smoke compartments. The ILSM is a health and safety measure to protect the one or more individuals at the facility. For determining the plurality of ILSMs, the ILSM determining subsystem is configured to select the plurality of ILSMs from the database, based on deficiencies associated with the one or more failed inspection points and the plurality of correlating deficiency assets.

The plurality of subsystems further comprises an asset controlling subsystem configured to configure the information associated with the selected plurality of ILSM actions with a plurality of internet of things (IoT) controllers for adapting the plurality of IoT controllers to automatically control the one or more assets within each of the one or more smoke compartments to protect the one or more individuals in the one or more smoke compartments of the facility. Each IoT controller of the plurality of IoT controllers is configured in corresponding one or more assets within each of the one or more smoke compartments. The plurality of subsystems further comprises an output subsystem configured to provide one or more controlled activities of the one or more assets within each of the one or more smoke compartments, to one or more users through one or more user interfaces associated with one or more electronic devices of the one or more users.

In an embodiment, for determining the plurality of risk levels for each of the one or more smoke compartments, based on the analyzed plurality of potential risk factors, the risk level determining subsystem is configured to: (a) analyze one or more correlation patterns between the plurality of potential risk factors and historical fire incident data to determine risk weighting coefficients for each potential risk factor within each of the one or more smoke compartments; (b) apply the one or more trained AI-based machine learning models comprising at least one of: neural networks, decision trees, and regression models to process the analyzed plurality of potential risk factors and generate quantitative risk assessment values for each of the one or more smoke compartments; (c) categorize the quantitative risk assessment values into the plurality of risk levels comprising at least one of: low risk, moderate risk, high risk, and critical risk levels based on predetermined threshold ranges; (d) incorporate environmental context factors comprising at least one of: occupancy type, building age, construction materials, and facility usage patterns to adjust the plurality of risk levels for each of the one or more smoke compartments; (e) perform dynamic risk level re-computation in real-time based on changes in the real-time sensor data and updated potential risk factors to maintain current risk level assessments; (f) validate risk level accuracy by comparing determined risk levels against historical incident patterns and regulatory compliance standards for analogous smoke compartments; and (g) generate risk level confidence scores indicating the reliability of each determined risk level based on data quality, sensor accuracy, and completeness of the analyzed plurality of potential risk factors.

In another embodiment, for classifying the one or more assets associated with each of the one or more smoke compartments into the plurality of risk levels, the asset classifying subsystem is configured to: (a) retrieve asset inventory data comprising asset identifiers, asset specifications, installation dates, and maintenance history for each of the one or more assets within each of the one or more smoke compartments from the database; (b) determine asset criticality levels by analyzing functional importance of each of the one or more assets to life safety operations within the corresponding one or more smoke compartments using predefined criticality matrices; (c) map asset locations to specific zones within each of the one or more smoke compartments using coordinate data, floor plans, and spatial relationship algorithms to establish the location environment for each asset; (d) apply the asset class inheritance-based AI models to automatically inherit risk characteristics from parent asset categories and propagate risk classifications to child assets based on hierarchical asset relationships; (e) execute the asset class acquisition-based AI models to dynamically acquire new risk classifications by analyzing real-time performance data and environmental conditions affecting each of the one or more assets; (f) correlate the one or more asset classes with the plurality of risk levels associated with each smoke compartment by matching asset safety functions, failure impact potential, and regulatory compliance requirements to the determined plurality of risk levels; (g) assign weighted risk factors to each of the one or more assets based on proximity to high-risk areas, interdependencies with other critical assets, and potential cascade failure effects within the smoke compartments; (h) validate asset classifications by cross-referencing determined asset risk levels against regulatory standards, manufacturer specifications, and historical failure patterns for similar assets; (i) generate asset risk profiles comprising asset class, assigned risk level, location environment factors, and classification confidence scores for each of the one or more assets; and (j) update asset classifications dynamically in response to changes in smoke compartment risk levels, asset performance degradation, or modifications to the location environment within the smoke compartments.

In yet another embodiment, for determining the aggregated score of the plurality of risk assessment scores associated with the one or more failed inspection points, the aggregated score determining subsystem is configured to: (a) identify the one or more failed inspection points by retrieving inspection results data and comparing actual inspection outcomes against required element of performance (EP) standards for each of the one or more inspection points within each of the one or more smoke compartments; (b) filter the plurality of risk assessment scores to isolate the plurality of risk assessment scores corresponding to the one or more failed inspection points during excluding scores from the one or more inspection points that passed the inspection; (c) apply one or more aggregation models comprising at least one of: weighted summation, root mean square computations, and maximum value selection, to combine the plurality of risk assessment scores associated with the one or more failed inspection points within each smoke compartment; (d) incorporate smoke compartment weighting factors based on compartment size, occupancy levels, and critical function designations to adjust the aggregated score computation for each of the one or more smoke compartments; (e) determine cumulative risk impact by analyzing the combined effect of the plurality of failed inspection points within the same smoke compartment, comprising potential synergistic effects amplifying overall risk levels; (f) apply temporal decay functions to adjust the plurality of risk assessment scores based on time elapsed as each inspection point failure was identified, wherein recent inspection point failures receive optimized weighting in the aggregated score determination; (g) normalize the aggregated scores across distinct smoke compartments to adapt consistent comparison and threshold evaluation regardless of compartment size and a number of the one or more assets contained within each compartment; (h) validate aggregated score accuracy by cross-referencing computed scores against historical incident data and regulatory risk assessment benchmarks for analogous facility types and smoke compartment configurations; and (i) generate aggregated score breakdown reports documenting the individual risk assessment scores, weighting factors, and computation methodologies used to determine a final aggregated score for each smoke compartment.

In yet another embodiment, for determining the plurality of ILSMs for each of the one or more assets within each of the one or more smoke compartments, the ILSM determining subsystem is configured to: (a) compare the aggregated score against the pre-determined threshold value for each of the one or more smoke compartments to identify the one or more smoke compartments requiring the plurality of ILSMs; (b) retrieve threshold configuration parameters from the database comprising pre-determined threshold values specific to distinct facility types, occupancy classifications, and regulatory requirements applicable to each of the one or more smoke compartments; (c) identify triggering assets by analyzing which of the one or more assets within each smoke compartment contributed the one or more failed inspection points that caused the aggregated score to exceed the pre-determined threshold value; (d) correlate the one or more failed inspection points with the plurality of correlating deficiency assets stored in the database to determine relationships between specific deficiencies and applicable ILSM actions; (e) select corresponding ILSM actions from the plurality of ILSM actions stored in the database based on the type of deficiencies, asset classifications, and smoke compartment characteristics associated with the one or more failed inspection points; (f) prioritize the plurality of ILSM actions based on severity of risk, regulatory compliance requirements, and potential impact on the one or more individuals within each of the one or more smoke compartments; (g) validate ILSM appropriateness by cross-referencing selected ILSM actions against regulatory standards, facility policies, and best practices for similar deficiency scenarios; (h) generate ILSM implementation plans comprising specific actions, required resources, implementation timelines, and responsible parties for each determined ILSM within each affected smoke compartment; (i) determine ILSM effectiveness metrics to estimate risk reduction achieved by implementing each determined ILSM action based on historical performance data and risk mitigation models; and (j) generate a report associated with ILSM determinations comprising justification for each selected ILSM action, expected duration of implementation, and criteria for ILSM termination when permanent corrections are completed.

In yet another embodiment, for configuring the information associated with the selected plurality of ILSM actions with the plurality of IoT controllers for adapting the plurality of IoT controllers to automatically control the one or more assets, the asset controlling subsystem is configured to: (a) identify IoT controller assignments by mapping each of the plurality of IoT controllers to corresponding one or more assets within each of the one or more smoke compartments based on asset location data and controller communication capabilities; (b) translate the plurality of ILSM actions into control commands by converting the selected plurality of ILSM actions into machine-readable instructions and control parameters compatible with the plurality of IoT controllers; (c) establish communication protocols between the asset controlling subsystem and the plurality of IoT controllers using at least one of: wireless communication, wired networks, and mesh networking topologies to enable real-time command transmission; (d) configure controller operating parameters by programming each IoT controller with specific control logic, safety thresholds, and automated response sequences corresponding to the determined ILSM actions for protecting the one or more individuals; (e) execute fail-safe mechanisms within each of the plurality of IoT controllers to determine whether safe operation and automatic reversion to safe states when communication failures and system malfunctions occur; (f) coordinate multi-controller operations by synchronizing actions between the plurality of IoT controllers when ILSM implementation requires coordinated control of interdependent assets across one or more areas of the smoke compartments; (g) monitor a status of the plurality of IoT controllers by continuously receiving operational feedback, error reports, and performance data from each of the plurality of IoT controllers to verify proper ILSM action execution; (h) validate control effectiveness by analyzing the real-time sensor data and asset performance metrics to determine that the automatically controlled assets are successfully protecting the one or more individuals as intended by the plurality of ILSM actions; (i) generate control audit logs documenting a plurality of commands sent to the plurality of IoT controllers, controller responses, and asset control actions performed for regulatory compliance and system troubleshooting purposes; and (j) update one or more configurations of the plurality of IoT controllers dynamically in response to changes in ILSM requirements, asset status modifications, and emergency conditions that require immediate adjustment of automated control parameters.

In yet another embodiment, the plurality of subsystems further comprises: (a) a work order generating subsystem configured to: generate at least one of one or more maintenance work orders and one or more corrective work orders, based on the determined plurality of ILSMs; and determine a status of the one or more maintenance work orders that is indicated as not completed and a status of the one or more corrective work orders that comprise one or more reported deficiencies requiring an immediate corrective action; (b) a profile retrieving subsystem configured to retrieve a risk profile corresponding to the location environment associated with each of the one or more assets in each of the one or more smoke compartments, from the database, wherein the risk profile comprises at least one of the potential risk factors, the plurality of risk levels, plurality of risk assessment scores, and the aggregated score. The work order creating subsystem is further configured to prioritize each of at least of: the one or more maintenance work orders and the one or more corrective work orders, based on the risk profile.

In yet another embodiment, the score assigning subsystem is further configured to: (a) determine at least one of: the one or more failed inspection points correspond to risk assessment assets, the location environment of the one or more assets corresponding to at least one of one or more supplementary facilities and one or more third party vendors, and a proximity to a subsequently discovered risk assessment assets; and (b) increment the risk assessment score in response to the determined risk assessment assets impacting the aggregated risk assessment score.

Another aspect of the present disclosure includes a computer-implemented method for determining an interim life safety measure (ILSM) to automatically control one or more assets within one or more smoke compartments in a facility. The computer-implemented method includes receiving, by one or more hardware processors associated with a computer-implemented system, real-time sensor data comprising at least one of: smoke density, temperature, humidity, environmental factors and image data captured from the one or more smoke compartments of the facility. The computer-implemented method further includes analyzing, by the one or more hardware processors, a plurality of potential risk factors associated with one or more individuals in the one or more smoke compartments of the facility, based on a type of the one or more smoke compartments. The individuals may include, but are not limited to, health care workers, workers, labors, patients, staff, health care providers, vendors, technicians, maintenance staff, visitors, and the like.

Analyzing the plurality of potential risk factors, comprises utilizing, by the one or more hardware processors, one or more trained Artificial Intelligence (AI)-based machine learning models, by at least one of: (a) detecting, by the one or more hardware processors, at least one of: smoke and flames in the one or more smoke compartments, based on large datasets of smoke and non-smoke image data; (b) analyzing, by the one or more hardware processors, the real-time sensor data from at least one of: temperature and humidity sensors, to predict fire risks in the one or more smoke compartments; (c) modeling, by the one or more hardware processors, causal relationships between one or more fire risk factors contributing to the fire risks in the one or more smoke compartments; (d) classifying, by the one or more hardware processors, the sensor data patterns comprising variations in temperature, humidity, and smoke density, indicative of the fire risks in the one or more smoke compartments; and (e) providing, by the one or more hardware processors, recommendations for at least one of: preventive measures and emergency response based on sensor data patterns associated with potential fire risks. The one or more trained AI-based machine learning models comprise at least one of: first convolutional neural networks (CNNs), recurrent neural networks (RNNs), Bayesian networks, first support vector machines (SVMs), and decision trees.

Further, the computer-implemented method further includes determining, by the one or more hardware processors, a plurality of risk levels for each of the one or more smoke compartments, based on the analyzed plurality of potential risk factors. Furthermore, the computer-implemented method further includes, in response to determining the plurality of risk levels, classifying, by the one or more hardware processors, one or more assets associated with each of the one or more smoke compartments into the plurality of risk levels, based on at least one of one or more asset classes and a location environment associated with each of the one or more assets in each of the one or more smoke compartments comprising the plurality of risk levels. The one or more asset classes are generated using at least one of: asset class inheritance-based AI models and asset class acquisition-based AI models. At least one of: the asset class inheritance-based AI models and asset class acquisition-based AI models is trained to: (a) analyze image data and historical data associated with the one or more assets; and (b) classify the one or more assets into asset classes based on the analyzed image data and historical data. At least one of the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, and Generative Adversarial Networks (GANs).

Additionally, the computer-implemented method further includes assigning, by the one or more hardware processors, a plurality of risk assessment scores to each of one or more inspection points corresponding to the classified one or more assets. The risk assessment score corresponds to at least one of an importance and a potential harm created when the one or more inspection point fails an inspection. Each of the one or more inspection points corresponds to a requirement of an element of performance (EP).

Further, the computer-implemented method includes determining, by the one or more hardware processors, an aggregated score of the plurality of risk assessment scores associated with one or more failed inspection points for the one or more assets within each of the one or more smoke compartments. The computer-implemented method further includes storing, by the one or more hardware processors, information associated with a plurality of Interim Life Safety Measure (ILSM) actions and a plurality of correlating deficiency assets, in a database. Each ILSM action defines an action related to protecting the one or more individuals at the facility. Each correlating deficiency asset defines a correlation between a deficiency encountered at the facility and at least one ILSM action.

Furthermore, the computer-implemented method includes determining, by the one or more hardware processors, a plurality of ILSMs for each of the one or more assets within each of the one or more smoke compartments, when the aggregated score is greater than a pre-determined threshold value for each of the one or more assets within each of the one or more smoke compartments. The ILSM is a health and safety measure to protect the one or more individuals at the facility. Determining the plurality of ILSMs comprises selecting, by the one or more hardware processors, the plurality of ILSMs from the database, based on deficiencies associated with the one or more failed inspection points and the plurality of correlating deficiency assets.

Additionally, the computer-implemented method includes configuring, by the one or more hardware processors, the information associated with the selected plurality of ILSM actions with a plurality of internet of things (IoT) controllers for adapting the plurality of IoT controllers to automatically control the one or more assets within each of the one or more smoke compartments to protect the one or more individuals in the one or more smoke compartments of the facility. Each IoT controller of the plurality of IoT controllers is configured in corresponding one or more assets within each of the one or more smoke compartments. Further, the computer-implemented method includes providing, by the one or more hardware processors, one or more controlled activities of the one or more assets within each of the one or more smoke compartments, as an output to one or more users through one or more user interfaces associated with one or more electronic devices of the one or more users.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates an exemplary block diagram representation of a network architecture for a computer-implemented system for determining an interim life safety measure (ILSM) to automatically control one or more assets within one or more smoke compartments in a facility, in accordance with an embodiment of the present disclosure;

FIG. 2 illustrates an exemplary block diagram representation of a detailed view of the computer-implemented system, in accordance with an embodiment of the present disclosure;

FIG. 3 illustrates an exemplary block diagram representation of an enterprise mobile, cloud-based compliance system to increase hospital facility management compliance and to improve management and maintenance of healthcare facilities, in accordance with an embodiment of the present disclosure;

FIG. 4 illustrates an exemplary flow diagram representation of an inspection method for compliance and risk assessment, in accordance with an embodiment of the present disclosure;

FIG. 5 illustrates an exemplary block diagram representation of elements of a compliance and risk assessment computer program, in accordance with an embodiment of the present disclosure;

FIG. 6 illustrates an exemplary flow diagram representation of a process for risk assessment in a compliance system, in accordance with an embodiment of the present disclosure;

FIG. 7 illustrates an exemplary flow diagram representation of a method for issuing an ILSM, in accordance with an embodiment of the present disclosure;

FIG. 8 illustrates a flow chart depicting a computer-implemented method for determining the interim life safety measure (ILSM) to automatically detect the one or more assets within the one or more smoke compartments in the facility, according to an example embodiment of the present disclosure; and

FIG. 9 illustrates an exemplary block diagram representation of a hardware platform for implementation of the disclosed computer-implemented system, according to an example embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

For simplicity and illustrative purposes, the present disclosure is described by referring mainly to examples thereof. The examples of the present disclosure described herein may be used together in different combinations. In the following description, details are set forth in order to provide an understanding of the present disclosure. It will be readily apparent, however, that the present disclosure may be practiced without limitation to all these details. Also, throughout the present disclosure, the terms “a” and “an” are intended to denote at least one example of a particular element. The terms “a” and “an” may also denote more than one example of a particular element. In the following specification and the claims, reference will be made to a number of terms, which shall be defined to have the following meanings. The singular forms “a,” “an,” and “the” include plural references unless the context clearly dictates otherwise.

As used herein, the term “includes” means includes but not limited to, the term “including” means including but not limited to. The term “based on” means based at least in part on, the term “based upon” means based at least in part upon, and the term “such as” means such as but not limited to. The term “relevant” means closely connected or appropriate to what is being performed or considered. The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements, structures, or components preceded by “comprises . . . a” does not, without more constraints, preclude the existence of other devices, subsystems, elements, structures, components, additional devices, additional subsystems, additional elements, additional structures, or additional components. Appearances of the phrase “in an embodiment”, “in another embodiment” and similar language throughout this specification may, but not necessarily do, all refer to the same embodiment.

Before describing in detail exemplary embodiments, it is noted that the embodiments reside primarily in combinations of components and procedures related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Unless otherwise defined, all technical and scientific terms used herein have the same meaning as commonly understood by those skilled in the art to which this disclosure belongs. The system, methods, and examples provided herein are only illustrative and not intended to be limiting.

Various embodiments of the present disclosure provide a computer-implemented system and method for determining an interim life safety measure (ILSM) to automatically control one or more assets in one or more smoke compartments in a facility. The smoke compartments are designated areas within a building that are separated from each other by fire-resistant barriers, such as walls, doors, floors, or ceilings, and the like, to prevent the spread of smoke and fire. The purpose of smoke compartments is to provide a safe means of egress for occupants of a building during a fire. In the event of a fire, smoke compartments help to contain smoke and heat, which can make it easier for firefighters to locate and extinguish the fire. They also allow for a safer evacuation of building occupants by preventing the spread of smoke and fire to other areas of the building. The smoke compartments are commonly found in high-rise buildings, hospitals, hotels, and other large structures. They are typically identified by signs or labels and are often included in building codes and fire safety regulations.

The present disclosure provides the computer-implemented system and method that affords hospitals the ability to assure ongoing compliance and reduce cost by migrating from their current existing costly manual inspections to using a central repository and tracking solution that helps improve healthcare physical environment quality of service and provides for successful compliance outcomes. Embodiments of the present disclosure ensure Interim Life Safety Measures (ILSMs) are completed, while also ensuring and validating the required activities are performed on time. Thus, embodiments of the present disclosure schedule regulatory compliance activities around regulatory mandated timelines. Embodiments of the present disclosure also provide warnings and alarms when deadlines approach, give emergency notifications to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall.

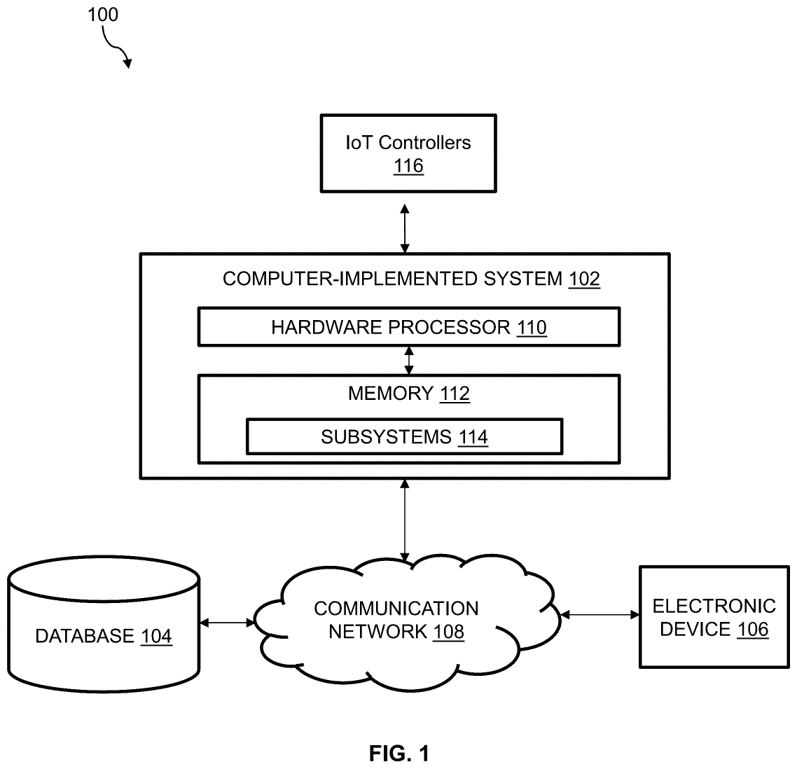

FIG. 1 illustrates an exemplary block diagram representation of a network architecture 100 for the computer-implemented system 102 for determining an interim life safety measure (ILSM) to automatically control the one or more assets within the one or more smoke compartments in the facility, in accordance with an embodiment of the present disclosure. The network architecture 100 may include the computer-implemented system 102, a database 104, and a user device 106. The computer-implemented system 102 may be communicatively coupled to the database 104, and the user device 106 via a communication network 108. The communication network 108 may be a wired communication network and/or a wireless communication network. The database 104 may include, but is not limited to, compartment data of a plurality of compartments for a facility, wherein each compartment defines a portion of the facility, risk assessment score for each inspection point, wherein the risk assessment score is a number indicating an importance and potential harm created when the inspection point fails inspection, a list of ILSM actions, correlating relationship data for correlating relationships between the types of deficiencies that may be encountered and the ILSM actions that address these deficiencies, any other content, and combinations thereof.

Further, the user device 106 may be associated with, but not limited to, a user, an administrator, a vendor, a technician, health care worker, a supervisor, compliance team, an entity, a facility, and the like. The user device 106 may be used to provide input and/or receive output to/from the system 102. The user device 106 may present to the user one or more user interfaces for the user to interact with the system 102 for the interim life safety measure (ILSM) needs.

The user device 106 may be at least one of, an electrical, an electronic, an electromechanical, and a computing device. The user device 106 may include, but is not limited to, a mobile device, a smart phone, a Personal Digital Assistant (PDA), a tablet computer, a phablet computer, a wearable computing device, a Virtual Reality/Augmented Reality (VR/AR) device, a laptop, a desktop, a server, and the like. The entities and the facility may include, but are not limited to, a hospital, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like.

Further, the computer-implemented system 102 may be implemented by way of a single device or a combination of multiple devices that may be operatively connected or networked together. The computer-implemented system 102 may be implemented in hardware or a suitable combination of hardware and software. The system 102 includes a hardware processor 110 and a memory 112. The memory 112 may include a plurality of subsystems 114.

The computer-implemented system 102 may be a hardware device including the hardware processor 110 executing machine-readable program instructions for determining an interim life safety measure (ILSM) for one or more smoke compartments in a facility. The one or more smoke compartments includes, but are not limited to, high-rise buildings, hospitals, hotels, office buildings, residential buildings, and the like. The high-rise building are the buildings that are taller than six stories usually require smoke compartments to provide a safe means of egress during a fire. Further, the smoke compartments in hospitals are used to separate patient care areas from other areas of the building, such as administrative offices, to prevent the spread of smoke and fire.

Furthermore, large hotels often have smoke compartments to separate guest rooms from other areas of the building, such as lobbies and restaurants. The smoke compartments can be used in office buildings to separate different departments or floors from each other, which can help prevent the spread of smoke and fire. The smoke compartments can be used in apartment buildings to separate individual units from each other, which can help prevent the spread of smoke and fire. Any building that requires multiple areas to be separated from each other to prevent the spread of smoke and fire may have smoke compartments.

Execution of the machine-readable program instructions by the hardware processor 110 may enable the proposed computer-implemented system 102 to determine an interim life safety measure (ILSM) in order to control the one or more assets within the one or more smoke compartments in the facility, using one or more internet of things (IoT) devices that are configured in each of the one or more assets. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The one or more hardware processors 110 may include, for example, microprocessors, microcomputers, microcontrollers, digital signal processors, central processing units, state machines, logic circuits, and/or any devices that manipulate data or signals based on operational instructions. Among other capabilities, one or more hardware processors 110 may fetch and execute computer-readable instructions in a memory operationally coupled with the computer-implemented system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, assets, machinery, instruments, facility equipment, optional equipment, facility condition inspection equipment, heating, ventilation and air-conditioning systems (HVAC), environmental care devices, life safety devices, intensive care devices, treatment devices, emergency management devices, health care device, door closures, and the like. The person skilled in the art should not be limiting the components/subsystems shown in FIG. 1.

In an exemplary embodiment, the computer-implemented system 102 may further execute the one or more hardware processors 110 to receive real-time sensor data comprising at least one of: smoke density, temperature, humidity, environmental factors and image data captured from the one or more smoke compartments of the facility.

The computer-implemented system 102 may further execute the one or more hardware processors 110 to analyze a plurality of potential risk factors associated with one or more individuals in the one or more smoke compartments of the facility, based on a type of the one or more smoke compartments. In an embodiment, for analyzing the plurality of potential risk factors, the computer-implemented system 102 may further execute the one or more hardware processors 110 to utilize one or more trained Artificial Intelligence (AI)-based machine learning models, by at least one of: (a) detecting at least one of: smoke and flames in the one or more smoke compartments, based on large datasets of smoke and non-smoke image data, (b) analyzing real-time data from at least one of: temperature and humidity sensors, to predict fire risks in the one or more smoke compartments, (c) modeling causal relationships between one or more fire risk factors contributing to a fire risk in the one or more smoke compartments, (d) classifying sensor data patterns comprising variations in temperature, humidity, and smoke density, indicative of a fire risk in the one or more smoke compartments, and (e) providing recommendations for at least one of: preventive measures and emergency response based on sensor data patterns associated with potential fire risks. In an embodiment, the one or more trained AI-based machine learning models comprise at least one of: first convolutional neural networks (CNNs), recurrent neural networks (RNNs), Bayesian networks, first support vector machines (SVMs), decision trees, and the like.

The computer-implemented system 102 may further execute the one or more hardware processors 110 to determine a plurality of risk levels for each of the one or more smoke compartments, based on the analyzed plurality of potential risk factors.

In an exemplary embodiment, the plurality of risk levels includes one or more dynamic values for each of the one or more asset classes, based on the location environment associated with each of the one or more assets. The individuals may include, but are not limited to, health care workers, workers, labors, patients, staff, health care providers, vendors, technicians, maintenance staff, visitors, and the like. For example, the one or more smoke compartments are designed to limit the spread of smoke within a building and protect occupants from the harmful effects of smoke inhalation. However, there are plurality of potential risk factors to one or more individuals in the one or more smoke compartments that can compromise their safety. Examples of potential riskfactors to one or more individuals include, but are not limited to, lack of oxygen, toxic smoke, physical obstructions, panic, injuries, and the like. Lack of oxygen: the smoke compartments are designed to contain smoke, but they can also contain oxygen. If the smoke compartment is not properly ventilated, occupants may experience a lack of oxygen, which can lead to dizziness, confusion, and unconsciousness. Toxic smoke: Smoke from a fire can be toxic, containing carbon monoxide, hydrogen cyanide, and other dangerous chemicals. If occupants are exposed to toxic smoke for an extended period, it can cause serious health problems and even death. Physical obstructions: the smoke compartments may be obstructed by debris or furniture, preventing occupants from exiting the compartment safely. Panic: In a fire emergency, occupants may panic, leading to disorientation and difficulty in finding the exits. Panic can also lead to poor decision-making and reckless behavior, putting occupants at greater risk. Injuries: Smoke compartments may contain hazards such as sharp objects, hot surfaces, or tripping hazards that can cause injuries to occupants.

Further, the risk levels include low risk, medium risk, high risk and highest risk level. The low risk level includes non-patient care areas, office areas, and plant area. Further, the medium risk level includes patient care support areas such as cardiology, echocardiography, endoscopy, fitness center, kitchen/cafeteria, lobby, nuclear medicine, outpatient area, radiology (all excluding magnetic resonance imaging (MRI)), radiology (MRI only), respiratory therapy rooms, and the like. Furthermore, the high-risk level includes patient care areas such as emergency room, inpatient unit, intensive care units, labor & delivery, laboratories (specimen), medical units, negative pressure isolation rooms, newborn nursery, oncology, operating rooms including c-section, orthopedics, outpatient surgery, physical therapy, and the like. Furthermore, the highest risk level includes procedural, invasive, sterile support and highly compromised patient care areas such as any area caring for immunocompromised patients, burn unit cardiac cath/electrophysiology lab, central sterile supply, critical care unit, pediatrics, pharmacy (incl retail pharmacy), post-anesthesia care unit, surgical units, and the like.

In an exemplary embodiment, the computer-implemented system 102 may execute the one or more hardware processors 110 to, in response to determining the plurality of risk levels, classify one or more assets associated with each of the one or more smoke compartments into the plurality of risk levels, based on at least one of: the one or more asset classes and the location environment associated with each of the one or more assets in each of the one or more smoke compartments comprising the plurality of risk levels. In an embodiment, the one or more asset classes are generated using at least one of: asset class inheritance-based AI models and asset class acquisition-based AI models. In an embodiment, at least one of: the asset class inheritance-based AI models and asset class acquisition-based AI models is trained to: (a) analyze image data and historical data associated with the assets, and (b) classify the assets into asset classes based on the analyzed image data and historical data. In an embodiment, at least one of the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, Generative Adversarial Networks (GANs), and the like.

The computer-implemented system 102 may further execute the one or more hardware processors 110 to assign a plurality of risk assessment scores to each of one or more inspection points corresponding to the classified one or more assets. In an exemplary embodiment, the risk assessment score corresponds to at least one of an importance and a potential harm created when the one or more inspection point fails an inspection. Each of the one or more inspection points corresponds to a requirement of an element of performance (EP). In an exemplary embodiment, the risk assessment is not performed for a failed inspection point when the risk assessment score associated with the failed inspection point is zero. In an exemplary embodiment, the risk assessment for a failed inspection point is delayed for a predetermined time when a “time to resolve” option is selected for a failed inspection point, and wherein risk assessment for the failed inspection point is performed when the failed inspection point is not completed within the predefined time.

The computer-implemented system 102 may further execute the one or more hardware processors 110 to determine an aggregated score of the plurality of risk assessment scores associated with one or more failed inspection points for the one or more assets within each of the one or more smoke compartments. The computer-implemented system 102 may further execute the one or more hardware processors 110 to store information associated with a plurality of Interim Life Safety Measure (ILSM) actions and a plurality of correlating deficiency assets, in the database 104. In an embodiment, each ILSM action defines an action related to protecting the one or more individuals at the facility. In another embodiment, each correlating deficiency asset defines a correlation between a deficiency encountered at the facility and at least one ILSM action.

The computer-implemented system 102 may further execute the one or more hardware processors 110 to determine one or more ILSMs (otherwise referred as a plurality of ILSMs), when the aggregated score is greater than a pre-determined threshold value for each of the one or more assets within each of the one or more smoke compartments. The ILSM is a health and safety measure to protect the one or more individuals at the facility. For determining the plurality of ILSMs, the computer-implemented system 102 is configured to select the plurality of ILSMs from the database 104, based on deficiencies associated with the one or more failed inspection points and the plurality of correlating deficiency assets.

The computer-implemented system 102 may further execute the one or more hardware processors 110 to configure the information associated with the selected plurality of ILSM actions with a plurality of internet of things (IoT) controllers 116, within each of a plurality of IoT devices, for adapting the plurality of IoT controllers to automatically control the one or more assets within each of the one or more smoke compartments to protect the one or more individuals in the one or more smoke compartments of the facility

The computer-implemented system 102 may further execute the one or more hardware processors 110 to provide one or more controlled activities of the one or more assets within each of the one or more smoke compartments, to one or more users through one or more user interfaces associated with one or more electronic devices 106 of the one or more users.

Optionally, the computer-implemented system 102 may further execute the one or more hardware processors 110 to generate at least one of one or more maintenance work orders and one or more corrective work orders, based on the determined one or more ILSMs. The computer-implemented system 102 may further execute the one or more hardware processors 110 to generate the one or more notifications that the determined ILSM is no longer needed when at least one of the one or more maintenance work orders and the one or more corrective work orders associated with the generated ILSM are completed.

The computer-implemented system 102 may further execute the one or more hardware processors 110 to determine the status of at least one of the one or more maintenance work orders is indicated as not completed and the status of the one or more corrective work orders comprises one or more reported deficiencies requiring an immediate corrective action.

The computer-implemented system 102 may further execute the one or more hardware processors 110 to retrieve from the database 104, a risk profile corresponding to the location environment associated with each of the one or more assets in each of the one or more smoke compartments. In an exemplary embodiment, the risk profile includes, but not limited to, the potential risk factors, the plurality of risk levels, plurality of risk assessment scores, the aggregated score, and the like.

The computer-implemented system 102 may further execute the one or more hardware processors 110 to prioritize each of at least of the one or more maintenance work orders and the one or more corrective work orders, based on the risk profile.

The computer-implemented system 102 may further execute the one or more hardware processors 110 to determine, when the failed inspection points correspond to risk assessment assets, the location environment of the one or more assets corresponding to at least one of one or more supplementary facilities and one or more third party vendors, and a proximity to a subsequently discovered risk assessment assets.

The computer-implemented system 102 may further execute the one or more hardware processors 110 to increment the risk assessment score, in response to the determined risk assessment assets impacting the aggregated risk assessment score.

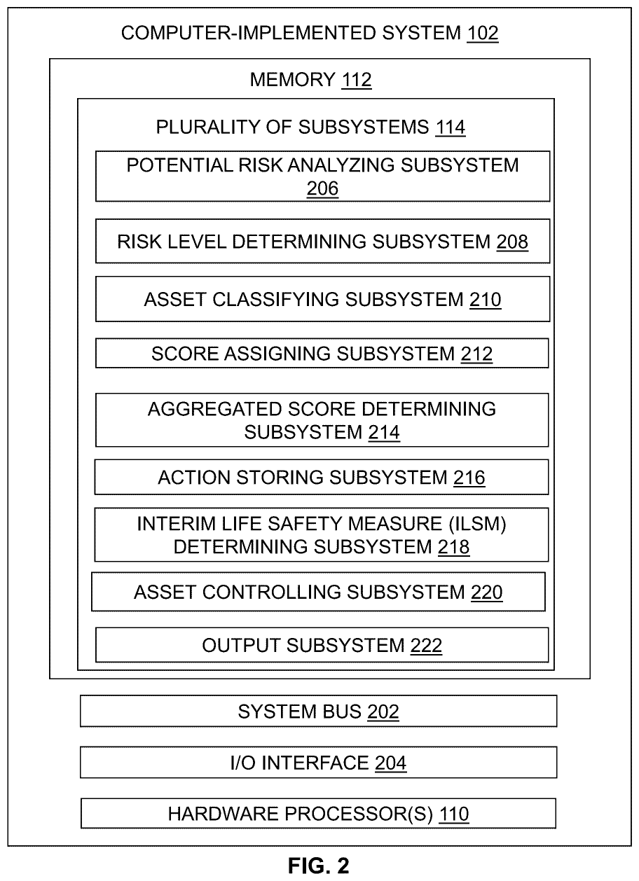

FIG. 2 illustrates an exemplary block diagram representation of a detailed view of the computer-implemented system 102, in accordance with an embodiment of the present disclosure. The computer-implemented system 102 includes the one or more hardware processors 110. The computer-implemented system 102 also includes a memory 112 coupled to the one or more hardware processors 110. The memory 112 includes a set of program instructions in the form of the plurality of subsystems 114.

The one or more hardware processor(s) 110, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 112 includes the plurality of subsystems 104 stored in the form of executable program which instructs the one or more hardware processors 110 via a system bus 202 to perform the above-mentioned method steps. Further, the system 102 may include an Input/Output (I/O) interface 204, which may be used to receive user inputs, from the user devices 106 associated with the users (not shown), and store/retrieve data from the database 104.

Further, the system 102 includes a memory 112 to store executable program and the plurality of subsystems 114. The plurality of subsystems 114 include a potential risk analyzing subsystem 206, a risk level determining subsystem 208, an asset classifying subsystem 210, a score assigning subsystem 212, an aggregated score determining subsystem 214, an action storing subsystem 216, an Interim Life Safety Measure (ILSM) determining subsystem 218, an asset controlling subsystem 220, and an output subsystem 222.

The plurality of subsystems 114 may be stored within the memory 112. In an example, the plurality of subsystems 114 communicatively coupled to the one or more hardware processors 110 configured in the system 102, may also be present outside the memory 112, and implemented as hardware. As used herein, the term “subsystems” may refer to an Application-Specific Integrated Circuit (ASIC), an electronic circuit, a processor (shared, dedicated, or group) and memory that execute one or more software or firmware programs, a combinational logic circuit, and/or other suitable components that provide the described functionality.

Further, the computer-implemented system 102 may also include other units such as a display unit, an input unit, an output unit, and the like, however the same are not shown in FIG. 1 and FIG. 2, for the purpose of clarity. Also, in FIG. 2 only a few units are shown, however, the computer-implemented system 102 or the network architecture 100 may include multiple such units or the computer-implemented system 102/network architecture 100 may include any such numbers of the units, obvious to a person skilled in the art or as required to implement the features of the present disclosure.

Computer memory elements may include any suitable memory device(s) for storing data and executable program, such as read only memory, random access memory, erasable programmable read only memory, electronically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 110.

The plurality of subsystems 114 includes the potential risk analyzing subsystem 206 that is communicatively connected to the one or more hardware processors 110. The potential risk analyzing subsystem 206 is configured to receive the real-time sensor data comprising at least one of: smoke density, temperature, humidity, environmental factors and image data captured from the one or more smoke compartments of the facility. The smoke density refers to the concentration or thickness of smoke particles present in the air within a smoke compartment, typically measured in units such as optical density per meter or percentage obscuration, which indicates the level of visibility impairment and potential fire hazard. For example, a smoke density reading of 5% obscuration per meter would indicate light smoke presence, while readings above 50% would represent dense smoke conditions requiring immediate evacuation protocols. The temperature refers to the ambient air temperature within the smoke compartment measured in degrees Celsius or Fahrenheit, which serves as a critical indicator of fire development, heat buildup, and potential thermal hazards to occupants. For example, normal room temperature of 20-25° C. (68-77° F.) would be considered safe, while temperatures exceeding 60° C. (140° F.) would indicate dangerous conditions requiring immediate safety measures. The humidity refers to the amount of water vapor present in the air within the smoke compartment, typically expressed as relative humidity percentage, which can affect fire behavior, smoke dispersion patterns, and the effectiveness of fire suppression systems. For example, normal indoor humidity levels of 30-50% relative humidity support optimal fire safety system performance, while extremely low humidity below 20% may increase fire spread risk and extremely high humidity above 80% may impair smoke detection sensitivity. The environmental factors refer to additional atmospheric and physical conditions within the smoke compartment that may influence fire risk and safety, including air pressure differentials, air flow patterns, ventilation rates, lighting conditions, and atmospheric contaminants. For example, negative air pressure in a smoke compartment may help contain smoke spread, while positive pressure could cause smoke migration to adjacent areas, or poor ventilation rates below 6 air changes per hour in healthcare facilities may allow smoke accumulation. The image data refers to visual information captured by cameras, thermal imaging devices, or other optical sensors within the smoke compartments, providing real-time visual monitoring capabilities for detecting smoke, flames, occupant presence, and emergency conditions. For example, visible light cameras may capture images showing smoke plumes or flame visibility, while thermal imaging cameras may detect heat signatures indicating fire development or occupant locations even in smoke-obscured environments.

The potential risk analyzing subsystem 206 is further configured to analyze the plurality of potential risk factors associated with the one or more individuals in the one or more smoke compartments of the facility, based on the type of the one or more smoke compartments. In an exemplary embodiment, the plurality of potential risk factors may be analyzed using at least one of, but not limited to, an artificial intelligence (AI) based smoke detectors, a predictive maintenance based AI model, Real-time monitoring based AI model, emergency response based AI model, occupant tracking based AI model, and the like.

For example, smoke detection systems: AI-powered smoke detectors can be trained to differentiate between different types of smoke and alert building managers to potential fire risks. These systems can use machine learning algorithms to analyze data on the composition and behavior of smoke and identify patterns that indicate a potential fire. In predictive maintenance, the AI can be used to analyze data on the condition and performance of building systems such as HVAC and electrical systems. By identifying patterns of failure and predicting equipment malfunctions, AI can help building managers schedule preventive maintenance and reduce the risk of equipment failure and fire. Furthermore, for real-time monitoring, the AI-powered sensors can be installed throughout a building to monitor temperature, humidity, and other environmental factors. These sensors can be linked to a central AI system that can analyze the data in real-time and provide alerts to building managers when conditions are conducive to a fire. Additionally, using emergency response systems, the AI can be used to coordinate emergency response efforts in the event of a fire. For example, AI-powered systems can analyze data on the location of the fire, the number of occupants in the building, and the location of emergency responders to provide real-time information to emergency responders and help them respond quickly and efficiently. Further, for occupant tracking, the AI-powered systems can track the location of occupants in a building and provide real-time information on their movements. In the event of a fire, this information can be used to locate and evacuate occupants quickly and safely.

In another aspect, for analyzing the plurality of potential risk factors, the potential risk analyzing subsystem 206 is configured to utilize the one or more trained AI-based machine learning models, by at least one of: (a) detecting at least one of: smoke and flames in the one or more smoke compartments, based on large datasets of smoke and non-smoke image data, (b) analyzing real-time data from at least one of: temperature and humidity sensors, to predict fire risks in the one or more smoke compartments, (c) modeling causal relationships between one or more fire risk factors contributing to a fire risk in the one or more smoke compartments, (d) classifying sensor data patterns comprising variations in temperature, humidity, and smoke density, indicative of a fire risk in the one or more smoke compartments, and (e) providing recommendations for at least one of: preventive measures and emergency response based on sensor data patterns associated with potential fire risks.

Examples of AI based models for analyzing potential risk factors to one or more individuals in smoke compartments include, but are not limited to, convolutional neural networks (CNNs), recurrent neural networks (RNNs), Bayesian networks, support vector machines (SVMs), decision trees, and the like. The CNNs are a type of deep learning model that can analyze images and video data to detect smoke or flames in a smoke compartment. These models are trained on large datasets of smoke and non-smoke images to learn to accurately classify smoke and alert building managers to potential fire risks. The RNNs are a type of deep learning model that can analyze data over time and make predictions about future events. These models can be used to analyze data from sensors in a smoke compartment to identify patterns that may indicate a fire risk. For example, an RNN model could analyze data from temperature or humidity sensors to predict when conditions in a smoke compartment are conducive to a fire. The Bayesian Networks are a probabilistic graphical model that can be used to model the causal relationships between different factors that contribute to a fire risk. For example, a Bayesian Network could model the relationships between factors such as the age of building systems, the presence of flammable materials, and the location of smoke detectors to identify areas of high fire risk. The SVMs are a type of machine learning model that can analyze data to identify patterns and classify data into different categories. SVMs can be used to analyze data from sensors in a smoke compartment to identify patterns that may indicate a fire risk, such as changes in temperature, humidity, or smoke density. Decision trees are a type of machine learning model that can analyze data and make predictions based on a series of binary decisions. These models can be used to analyze data from sensors in a smoke compartment to identify potential fire risks and provide recommendations for preventive measures or emergency response.

In an embodiment, the CNN utilizes hierarchical feature extraction through convolutional layers, pooling operations, and activation functions to automatically learn spatial patterns and visual characteristics from image data, making them exceptionally effective at detecting smoke and flames by identifying distinctive visual signatures such as smoke's translucent, billowing patterns and flames' bright, flickering appearances. For example, the CNN trained on 100,000 smoke and non-smoke images can achieve 95% accuracy in detecting smoke plumes in hospital corridors by learning to recognize subtle visual cues like opacity gradients, edge patterns, and color variations that distinguish smoke from steam, dust, or normal air. The CNNs process multi-dimensional sensor data (i.e., analyzing the real-time sensor data) representations such as 2D heat maps, time-frequency spectrograms, or spatial-temporal matrices to identify complex patterns across multiple sensors (i.e., temperature and humidity sensors) and time periods. For example, a CNN analyzing a 24-hour temperature-humidity matrix from 50 sensors can detect localized anomalies (i.e., prediction of the fire risks) like a 5° C. temperature spike in sector 3 combined with 15% humidity drop, indicating potential fire development in that specific area. The CNNs learn complex non-linear interactions (i.e., modeling causal relationships) between multiple risk factors by processing multi-channel input representations that combine various risk indicators into unified feature maps. For example, a CNN processing 10-channel input combining electrical load, temperature, humidity, air flow, material combustibility, occupancy, maintenance status, equipment age, ventilation efficiency, and historical incident data can learn that electrical overload+poor ventilation+combustible materials creates a specific pattern signature indicating 90% fire probability. The CNNs classify multi-dimensional sensor patterns by processing concatenated sensor data (i.e., variations in temperature, humidity, and smoke density) as image-like representations or multi-channel feature maps. For example, a CNN analyzing a 3D tensor combining temperature (channel 1), humidity (channel 2), and smoke density (channel 3) over 100 time steps can classify patterns like “gradual_onset” (slow temperature rise), “flash fire” (rapid multi-sensor spike), or “false_alarm” (single sensor anomaly). The CNNs generate recommendations for at least one of: preventive measures (proactive safety actions, protocols, equipment deployments, and system modifications implemented before fire incidents occur to reduce fire risk, eliminate hazardous conditions, improve detection capabilities, enhance suppression readiness, and protect facility occupants from potential fire-related harm) and emergency response (immediate coordinated actions, procedures, and interventions implemented during active fire incidents or safety emergencies to protect lives, control hazards, facilitate evacuations, and minimize damage.) by processing complex multi-modal inputs including current sensor data, facility layouts, occupancy information, and equipment status to output appropriate response actions. For example, a CNN analyzing current fire indicators, building blueprints, and real-time occupancy data might recommend “Activate suppression zone 3, evacuate sectors 3-5 via east stairwell, deploy fire teams to floors 2-4, isolate HVAC system” based on learned associations between fire patterns and optimal response strategies.

The RNNs process sequential image frames through memory cells and recurrent connections to capture temporal dynamics and movement patterns over time, enabling detection of smoke and flame development through motion analysis and temporal feature evolution. For example, an RNN analyzing video sequences can detect the characteristic upward movement and expansion of smoke plumes or the dynamic flickering patterns of flames by tracking pixel intensity changes across 30 consecutive frames captured at 1-second intervals. The RNNs excel at processing sequential sensor measurements (i.e., real-time sensor data from at least one of: temperature and humidity sensors) through LSTM or GRU cells to capture long-term dependencies and predict future fire risks based on temporal trends and patterns. For example, an RNN analyzing hourly temperature and humidity readings can predict fire risk by detecting patterns like “temperature increasing 2° C./hour for 4 consecutive hours while humidity decreases 5%/hour,” forecasting high fire probability within the next 2 hours. The RNNs capture temporal causal chains and delayed effects (i.e., modeling of causal relationships) between risk factors through recurrent connections that maintain memory of previous states. For example, an RNN might learn the causal sequence: “electrical fault at t=0→temperature rise at t=10 min→humidity decrease at t=20 min→ventilation system strain at t=30 min→critical fire risk at t=45 min,” enabling prediction of cascading failure scenarios. The RNNs classify temporal sensor data patterns by learning characteristic sequences that distinguish different fire development stages. For example, an RNN might classify the pattern [temp: 25→30→38→45° C., humidity: 50→45→35→25%, smoke: 0→2→8→15%] over 20 minutes as “INCIPIENT_FIRE_STAGE” versus [temp: 25→55° C., humidity: 50→20%, smoke: 0→40%] over 5 minutes as “RAPID_FIRE_DEVELOPMENT.” The RNNs provide time-aware recommendations by considering current conditions, predicted future states, and temporal context to suggest proactive measures (i.e., preventive measures and emergency response). For example, an RNN detecting early fire indicators might recommend “T+0 min: Increase ventilation sector 3, T+5 min: Alert fire watch team, T+10 min: Prepare suppression systems, T+15 min: Initiate evacuation if temperature exceeds 50° C.” based on predicted fire development timeline.

The Bayesian networks represent probabilistic relationships between visual evidence and fire indicators (i.e., smoke and flames in the one or more smoke compartments) through directed acyclic graphs with conditional probability distributions, combining multiple sources of visual evidence to determine fire detection confidence while handling uncertainty. For example, a Bayesian network might integrate image analysis showing 70% smoke probability with infrared signatures indicating 80% heat source probability to compute an overall fire detection confidence of 85% while accounting for false positive rates from steam or dust. The Bayesian networks model probabilistic relationships between environmental conditions and fire risk through conditional probability tables that incorporate domain expertise and historical data. For example, a network might determine the real-time sensor data i.e., temperature >40° C. has 30% base fire risk, but when combined with humidity <25%, the conditional probability increases to 75% based on learned relationships from 10,000 historical fire incidents. The Bayesian networks explicitly represent causal relationships (i.e., modeling of causal relationships) through directed graphs with nodes representing risk factors and edges showing causal influences, quantified by conditional probability distributions. For example, a network might show “Faulty_Wiring→Electrical_Overheating (P=0.8)→Ignition_Source (P=0.6)→Fire_Occurrence (P=0.9)” while “Poor_Ventilation→Heat_Accumulation (P=0.7)→Fire_Spread (P=0.8).” The Bayesian networks classify the sensor data patterns by calculating posterior probabilities for different fire risk categories given observed sensor evidence. For example, given evidence [temperature=42° C., humidity=28%, smoke_density=12%, air flow=low], the network might output probabilities: Normal=5%, Early_Fire=25%, Active_Fire=60%, False_Alarm=10%. The Bayesian networks generate recommendations for at least one of: preventive measures and emergency response by calculating expected utilities of different actions given current evidence and associated uncertainties. For example, given 65% fire probability, the network might recommend “Activate enhanced monitoring (utility=0.8), Deploy fire watch (utility=0.9), Prepare evacuation (utility=0.7)” while avoiding full evacuation (utility=0.3) due to high operational disruption cost versus moderate fire probability.

The Support Vector Machines classify image regions as smoke, flame, or normal by finding optimal hyperplanes in high-dimensional feature spaces derived from image characteristics such as color histograms, texture descriptors, and edge patterns. For example, an SVM trained on extracted features like red-channel intensity (>180 for flames), gray-scale variance (>0.3 for smoke), and edge density can classify image patches with 92% accuracy by separating fire-related visual patterns from normal environmental conditions. The SVMs analyzes (i.e., analyzing of the real-time sensor data) environmental conditions into risk categories by mapping temperature-humidity combinations to high-dimensional feature spaces where fire-risk and normal conditions become linearly separable. For example, an SVM might classify conditions with features [temperature=45° C., humidity=20%, rate_of_change=3° C./min] as “HIGH_RISK” while [temperature=25° C., humidity=55%, rate_of_change=0.1° C./min] as “LOW_RISK.” The SVMs identify complex interaction patterns between risk factors by mapping them to high-dimensional kernel spaces where causal relationships (i.e., modeling causal relationships) become apparent through support vector boundaries. For example, an SVM using RBF kernel might discover that the combination [electrical_load=0.9, ventilation_efficiency=0.3, combustible_density=0.8] creates a distinct cluster in feature space associated with fire incidents, revealing hidden causal interactions. The SVMs classify sensor data patterns by finding optimal decision boundaries in multi-dimensional sensor feature space. For example, an SVM with polynomial kernel might classify sensor vectors where [temp>38° C. AND humidity<35% AND smoke>8% AND temp_gradient>2° C./min] as “FIRE_PATTERN” class versus vectors with stable readings as “NORMAL_PATTERN” class. The SVMs provide recommendations by classifying current conditions into predefined response categories with associated standard operating procedures. For example, an SVM classifying current sensor patterns as “RISK_LEVEL_3” might automatically trigger recommendations: “Increase patrol frequency to 15-minute intervals, Test suppression systems, Notify fire department standby, Restrict access to affected zones.”

The Decision Trees generate interpretable, rule-based classification systems using hierarchical threshold-based decisions on image features such as pixel intensities, color ratios, and texture measurements (i.e., detecting at least one of: smoke and flames). For example, a decision tree might follow rules like “if red_intensity>200 AND flickering_frequency >3 Hz then classify as FLAME; else if gray_opacity >0.4 AND upward_motion detected then classify as SMOKE; else classify as NORMAL.” The Decision Trees provide clear, interpretable rules for fire risk prediction (i.e., analyzing the real-time sensor data from at least one of: temperature and humidity sensors, to predict the fire risks) using threshold-based decisions on temperature and humidity values. For example, “if temperature >50° C. then HIGH_RISK; else if temperature >35° C. AND humidity <30% then MEDIUM_RISK; else if humidity <20% AND temperature_increase >5° C./hour then MEDIUM_RISK; else LOW_RISK.” The Decision Trees naturally represent causal logic (i.e., modeling of causal relationships) through hierarchical if-then rules that show how combinations of risk factors lead to specific outcomes. For example, “if Electrical_System_Age >20_years AND Maintenance_Frequency <6_months then check Load_Factor; if Load_Factor >0.8 then check Ventilation; if Ventilation=POOR then Fire_Risk=CRITICAL.” The Decision Trees classify sensor data patterns using interpretable threshold-based rules with clear decision paths. For example, “if smoke_density >15% then IMMEDIATE_FIRE; else if temperature >40° C. AND humidity <30% then DEVELOPING_FIRE; else if temperature_rate >3° C./min then EARLY_FIRE; else NORMAL.” The Decision Trees generate clear, rule-based recommendations that facility personnel can easily understand and execute. For example, “if fire_risk=CRITICAL then RECOMMEND: [Immediate evacuation+Activate all suppression+Call fire department+Isolate utilities]; if fire_risk=HIGH then RECOMMEND: [Prepare evacuation+Activate local suppression+Alert fire department+Increase monitoring]; if fire_risk=MEDIUM then RECOMMEND: [Enhanced monitoring+Test systems+Brief staff+Document conditions].”

The plurality of subsystems 114 further includes the risk level determining subsystem 208 that is communicatively connected to the one or more hardware processors 110. The risk level determining subsystem 208 is configured to determine a plurality of risk levels for each of the one or more smoke compartments, based on the analyzed plurality of potential risk factors. The risk level determining subsystem 208 performs determination by processing the analyzed plurality of potential risk factors through algorithmic assessment methodologies (e.g., Weighted Linear Combination Algorithm, Fuzzy Logic Assessment Algorithm, Multi-Criteria Decision Analysis (MCDA) Algorithm, Machine Learning Ensemble Algorithm, and the like) to generate multiple categorized risk levels for each smoke compartment, wherein the risk level determining subsystem 208 applies weighted scoring algorithms that combine quantified risk factors such as smoke density readings (e.g., 12% obscuration), temperature measurements (e.g., 45° C.), humidity levels (e.g., 25% RH), and AI-analyzed image data indicating flame presence to determine composite risk scores, then categorizes these scores into a plurality of distinct risk levels such as “Immediate Fire Risk” (score 85/100), “Evacuation Risk” (score 70/100), “Equipment Damage Risk” (score 60/100), and “Regulatory Compliance Risk” (score 40/100) for each smoke compartment. For example, Smoke Compartment A in the ICU wing might receive risk level determinations of [Critical Immediate Fire Risk, High Evacuation Risk, Moderate Equipment Risk, Low Compliance Risk] based on analyzed factors showing high smoke density (15% obscuration), elevated temperature (48° C.), low humidity (22%), and CNN-detected flame signatures, while Smoke Compartment B in the storage area receives [Low Immediate Fire Risk, Low Evacuation Risk, High Equipment Risk, Moderate Compliance Risk] based on normal environmental readings but failed sprinkler system inspection points, thereby enabling the risk level determining subsystem 208 to generate multiple dimensional risk assessments that reflect different safety concerns and operational priorities for each specific smoke compartment location and function within the facility.

In an exemplary embodiment, the plurality of risk levels includes one or more dynamic values for each of the one or more asset classes, based on the location environment associated with each of the one or more assets. For example, one or more asset classes in hospital smoke compartments can include various types of equipment and furnishings that are required for patient care and safety, heating, ventilation and air-conditioning systems (HVAC) system, fire systems (alarms, sprinklers), electrical system, fire doors, other mechanical systems present in the room.

For instance, medical equipment, such as patient monitors, infusion pumps, ventilators, and dialysis machines, are essential for patient care and can be considered an asset class in hospital smoke compartments. The furniture and fixtures such as Hospital beds, exam tables, chairs, and other furnishings used in patient rooms and treatment areas are also considered an asset class within smoke compartments. An emergency equipment, such as fire extinguishers, fire alarms, and emergency lighting, are critical components of hospital smoke compartments and are necessary to ensure the safety of patients, staff, and visitors. The building infrastructure, including heating, ventilation and air-conditioning systems (HVAC) systems, electrical systems, and plumbing systems, can also be considered an asset class within smoke compartments, as these systems must be designed and maintained to prevent the spread of smoke and fire. A personal protective equipment (PPE) such as masks, gloves, and gowns, are essential for preventing the spread of infectious diseases in hospitals and can also be considered an asset class within smoke compartments.

For example, the plurality of risk levels for the one or more assets may be based on inheritance or acquisition. The inheritance of the plurality of risks/risk levels are based on the one or more asset classes. Further, acquisition of the risks/risk levels is based on the location environment of the asset classes. The plurality of risk levels are not a static value for an asset type, it can be higher or lower for same asset, based on the asset location. For instance, a heating, ventilation and air-conditioning systems (HVAC) unit in a negative pressure room may include risk score=very high while the risk level for the exact same asset when the HVAC location is in a cafeteria or lobby is a score=Low. When an inspection of an asset fails, the computer-implemented system 102 may consider addition risk assessment, a risk profile based on the location environment of the assets.

The risk profile includes different risks for different environments, for example, negative-air pressure rooms, emergency rooms (ER), and the like. The risk profile may be generated based on the type of asset/asset classes and possibility of harm/potential risk factors, based on location of assets, amount of risk in areas such as lobby vs. emergency rooms, different departments such as infectious diseases vs. emergency rooms vs. cafeteria, and the like.

Further, the inheritance or the acquisition for the asset class may be based on using inheritance or acquisition-based AI models. Such AI models may include convolutional neural networks (CNNs), random forests, SVM, long short-term memory (LSTM) networks, generative adversarial networks (GANs), and the like. For example, the CNNs are a type of deep learning model that can analyze images and classify them based on their visual features. For example, a CNN could be trained to classify real estate properties based on their architectural style, location, and age. The random forests are a type of decision tree model that can be used to classify data into multiple classes based on a combination of features. For example, a Random Forest model could be trained to classify stocks into different asset classes based on their historical performance, industry sector, and financial metrics. The SVMs are a type of machine learning model that can be used to classify data into different categories based on a set of features. For example, an SVM model could be trained to classify artworks into different asset classes based on their style, medium, and historical auction prices. The LSTMs are a type of recurrent neural network that can be used to analyze time-series data and make predictions about future values. For example, an LSTM model could be trained to predict the asset class of a stock based on its historical performance over time. The GANs are a type of deep learning model that can be used to generate new data samples that are similar to a given dataset. For example, a GAN model could be trained to generate new artworks that are similar in style to existing artworks in a particular asset class.

In an example, certain areas/location environment such as immunocompromised patients, surgery, intensive care, neonatal intensive care unit (NICU) may be assigned as very high-risk level. Further, certain areas/location environment such as front lobby waiting area or cafeteria may be assigned as low risk level.

In an aspect, for determining the plurality of risk levels for each of the one or more smoke compartments, based on the analyzed plurality of potential risk factors, the risk level determining subsystem 208 is configured to analyze one or more correlation patterns between the plurality of potential risk factors and historical fire incident data to determine risk weighting coefficients for each potential risk factor within each of the one or more smoke compartments. The risk level determining subsystem 208 analyzes correlation patterns by performing statistical analysis to identify relationships between current potential risk factors (such as smoke density, temperature, humidity, environmental conditions, and AI-processed image data) and historical fire incident data from similar facilities, then uses these correlations to determine risk weighting coefficients that quantify how much each risk factor contributes to fire probability within specific smoke compartments, enabling the determination of multiple risk levels by applying these weighted coefficients to current risk factor measurements. For example, if historical data shows that in ICU smoke compartments, temperature increases above 40° C. preceded 85% of fire incidents while humidity drops below 25% preceded only 45% of incidents, the risk level determining subsystem 208 assigns a higher weighting coefficient of 0.6 to temperature and 0.3 to humidity, then applies these weights to current readings (temperature 43° C.×0.6=25.8 points, humidity 22%×0.3=6.6 points) to calculate composite risk scores that are categorized into multiple risk levels such as “Immediate Fire Risk=High (32.4/40 points)”, “Evacuation Risk=Moderate”, and “Equipment Risk=Low” for that specific smoke compartment.

For determining the plurality of risk levels for each of the one or more smoke compartments, based on the analyzed plurality of potential risk factors, the risk level determining subsystem 208 is configured to apply the one or more trained AI-based machine learning models comprising at least one of: neural networks, decision trees, and regression models to process the analyzed plurality of potential risk factors and generate quantitative risk assessment values for each of the one or more smoke compartments. The risk level determining subsystem 208 applies trained AI-based machine learning models including neural networks (which use interconnected nodes to learn complex non-linear relationships), decision trees (which create rule-based branching logic), and regression models (which establish mathematical relationships between variables) to process the analyzed potential risk factors and automatically generate numerical risk assessment values that quantify fire danger levels for each smoke compartment. The neural networks process the analyzed plurality of potential risk factors by utilizing interconnected layers of artificial neurons that receive input data (such as smoke density readings, temperature variations, humidity levels, occupancy patterns, and historical incident data), apply weighted mathematical transformations through hidden layers that identify complex non-linear relationships and patterns between multiple risk factors simultaneously, and generate quantitative risk assessment values through output neurons that produce numerical scores representing fire risk probability for each smoke compartment; for example, when processing risk factors for ICU Smoke Compartment A including “high patient density: 24 patients, flammable oxygen concentration: 35%, building age: 15 years, maintenance score: 78%, recent temperature anomalies: 3 incidents,” the neural network applies learned weights through multiple hidden layers to identify that the combination of high patient density+elevated oxygen levels+moderate building age creates a complex risk interaction pattern, ultimately generating a quantitative risk assessment value of 8.7 out of 10.0 for this smoke compartment based on the network's training on thousands of similar facility scenarios and historical fire incident outcomes.

The decision trees process the analyzed plurality of potential risk factors by constructing hierarchical branching structures that systematically evaluate each risk factor through a series of binary decision nodes, where each branch represents a specific condition or threshold that splits the data based on risk factor values, following decision paths through multiple levels of criteria evaluation until reaching terminal leaf nodes that assign specific quantitative risk assessment values to each smoke compartment; for example, when evaluating ICU Smoke Compartment A, the decision tree follows the path “Is patient density >20?YES →Is oxygen concentration >30%?YES →Is building age >10 years?YES →Is maintenance score <80%?YES →Terminal Node: Assign quantitative risk assessment value=8.5,” thereby generating a clear, interpretable risk score of 8.5 out of 10.0 through logical decision sequences that demonstrate exactly which combination of risk factors led to this specific numerical rating. The regression models process the analyzed plurality of potential risk factors by establishing mathematical relationships between multiple input variables (independent risk factors such as building age, construction materials, occupancy density, maintenance history, and environmental conditions) and output variables (quantitative risk assessment values), using statistical algorithms to determine correlation coefficients and generate predictive equations that calculate numerical risk scores based on weighted combinations of risk factor values; for example, when processing ICU Smoke Compartment A risk factors, the regression model applies the learned equation “Risk Score=2.1×(Patient Density/10)+1.8×(Oxygen Level/100)+0.9×(Building Age/20)+1.2×(100−Maintenance Score)/100” to calculate Risk Score=2.1×(24/10)+1.8×(35/100)+0.9×(15/20)+1.2×(100−78)/100=5.04+0.63+0.675+0.264=6.6, generating a quantitative risk assessment value of 6.6 out of 10.0 for this smoke compartment based on the mathematical relationship between weighted risk factors.

For determining the plurality of risk levels for each of the one or more smoke compartments, based on the analyzed plurality of potential risk factors, the risk level determining subsystem 208 is configured to categorize the quantitative risk assessment values into the plurality of risk levels comprising at least one of: low risk, moderate risk, high risk, and critical risk levels based on predetermined threshold ranges. The risk level determining subsystem 208 categorizes the quantitative risk assessment values by applying predetermined threshold ranges that define specific boundaries for each risk level classification, wherein numerical risk scores generated by AI models are systematically assigned to discrete risk categories based on where they fall within established ranges, enabling consistent and standardized risk level determination across all smoke compartments. For example, if the predetermined threshold ranges are defined as Low Risk (0-25 points), Moderate Risk (26-50 points), High Risk (51-75 points), and Critical Risk (76-100 points), then a smoke compartment with a quantitative risk assessment value of 32.4 points would be categorized as “Moderate Risk,” while another compartment scoring 78.9 points would be categorized as “Critical Risk,” and a third compartment with 15.2 points would be classified as “Low Risk,” thereby converting continuous numerical risk scores into actionable categorical risk levels that facility managers can easily understand and use for decision-making regarding interim life safety measures and emergency response protocols.

For determining the plurality of risk levels for each of the one or more smoke compartments, based on the analyzed plurality of potential risk factors, the risk level determining subsystem 208 is configured to incorporate environmental context factors comprising at least one of: occupancy type, building age, construction materials, and facility usage patterns to adjust the plurality of risk levels for each of the one or more smoke compartments. The risk level determining subsystem 208 incorporates environmental context factors by retrieving and analyzing specific facility characteristics including occupancy type (classification of space usage and occupant vulnerability), building age (chronological age affecting system reliability and code compliance), construction materials (physical building components influencing fire behavior), and facility usage patterns (operational activities and occupancy variations), then applies contextual adjustment algorithms that modify the initially determined risk levels based on these factors to ensure accurate risk assessment reflecting real-world conditions. For example, a smoke compartment initially assessed as “Moderate Risk” (base score: 50/100) might be adjusted upward to “High Risk” (adjusted score: 72/100) when environmental context analysis reveals it has occupancy type=“intensive care unit” (+15 points for vulnerable patients), building age=“35 years old” (+10 points for aging systems), construction materials=“mixed combustible/non-combustible” (+5 points for partial fire resistance), and facility usage patterns=“24/7 high-acuity operations” (+7 points for continuous critical activities), while another compartment with identical sensor readings but environmental context of occupancy type=“storage room” (−5 points), building age=“5 years old” (−8 points), construction materials=“fully non−combustible” (−10 points), and facility usage patterns=“daytime intermittent use” (−3 points) would be adjusted downward to “Low Risk” (adjusted score: 24/100), demonstrating how environmental context factors provide essential modifications that ensure risk levels accurately reflect the unique safety challenges, regulatory requirements, and operational realities of each specific smoke compartment's physical and functional environment.

For determining the plurality of risk levels for each of the one or more smoke compartments, based on the analyzed plurality of potential risk factors, the risk level determining subsystem 208 is configured to perform dynamic risk level re-computation in real-time based on changes in the real-time sensor data and updated potential risk factors to maintain current risk level assessments. The risk level determining subsystem 208 performs dynamic risk level re-computation by continuously monitoring incoming real-time sensor data streams and updated potential risk factors (i.e., newly identified or modified hazards, vulnerabilities, and safety concerns that emerge or change over time due to facility modifications, equipment changes, environmental shifts, or operational adjustments that could affect fire risk levels), then automatically re-computing and updating risk level assessments whenever significant changes are detected to ensure that risk evaluations remain current and accurate as conditions evolve within the smoke compartments. For example, if a smoke compartment initially has a “Moderate Risk” level (score: 45/100) based on temperature=35° C., humidity=40%, and smoke density=3%, but real-time sensors detect rapid changes showing temperature rising to 48° C., humidity dropping to 25%, and smoke density increasing to 12% over a 10-minute period, the risk level determining subsystem 208 immediately triggers dynamic re-computation using the updated sensor values and revised potential risk factors (such as AI models detecting flame signatures in new image data), resulting in an updated risk level assessment of “Critical Risk” (new score: 85/100) that reflects the rapidly deteriorating conditions, with this re-computation process occurring continuously in real-time (every 30 seconds or upon threshold changes) to maintain current risk level assessments that accurately represent the evolving fire safety situation and enable immediate response to changing hazard conditions within each smoke compartment.

For determining the plurality of risk levels for each of the one or more smoke compartments, based on the analyzed plurality of potential risk factors, the risk level determining subsystem 208 is configured to validate risk level accuracy by comparing determined risk levels against historical incident patterns and regulatory compliance standards for analogous smoke compartments. The risk level determining subsystem 208 validates risk level accuracy by performing comparative analysis that cross-references the newly determined risk levels against established benchmarks including historical incident patterns (documented records of past fire events and their associated conditions) and regulatory compliance standards (official safety codes and requirements) for analogous smoke compartments (similar compartments with comparable characteristics, functions, and risk profiles), using statistical correlation analysis and compliance verification algorithms to ensure the determined risk levels are consistent with empirical evidence and regulatory expectations. For example, if the risk level determining subsystem 208 determines a “High Risk” level (score: 78/100) for an ICU smoke compartment based on current sensor readings of temperature=46° C., humidity=22%, and smoke density=14%, the validation process compares this determination against historical incident patterns showing that 85% of actual fires in similar ICU compartments occurred when risk scores exceeded 75/100, and against regulatory compliance standards such as NFPA 101 Life Safety Code requirements for healthcare occupancies that mandate enhanced protection measures when environmental conditions exceed specified thresholds, confirming that the “High Risk” determination is accurate and appropriate, while a compartment receiving “Low Risk” (score: 15/100) would be validated by showing that similar scores in analogous compartments historically correlated with only 2% fire incident probability and met all regulatory safety margins, thereby ensuring that all risk level determinations are supported by both empirical evidence from past incidents and compliance with established safety standards.

For determining the plurality of risk levels for each of the one or more smoke compartments, based on the analyzed plurality of potential risk factors, the risk level determining subsystem 208 is configured to generate risk level confidence scores indicating the reliability of each determined risk level based on data quality, sensor accuracy, and completeness of the analyzed plurality of potential risk factors. The risk level determining subsystem 208 generates risk level confidence scores by performing reliability assessment that evaluates three critical factors: data quality (accuracy, consistency, and validity of collected sensor measurements), sensor accuracy (precision and calibration status of monitoring equipment), and completeness of analyzed potential risk factors (percentage of required risk indicators successfully captured and processed), then combines these evaluations using weighted algorithms to produce numerical confidence scores that indicate the trustworthiness and reliability of each determined risk level. For example, if a smoke compartment receives a “High Risk” determination (score: 82/100) based on sensor data where data quality assessment shows 95% accuracy (temperature sensors recently calibrated, humidity readings consistent over time, no data corruption detected), sensor accuracy evaluation indicates 98% precision (all sensors within calibration tolerances, minimal measurement drift), and completeness analysis reveals 90% of potential risk factors captured (smoke density, temperature, humidity, image data all available, but air flow sensor offline), the risk level determining subsystem 208 would generate a high confidence score of 94% for this risk level determination, while another compartment with “Moderate Risk” determination but poor data quality (70% due to intermittent sensor failures), lower sensor accuracy (85% due to overdue calibrations), and incomplete risk factor analysis (60% due to multiple sensor outages) would receive a low confidence score of 72%, enabling facility managers to understand the reliability of each risk assessment and make informed decisions about whether additional verification or sensor maintenance is needed before implementing interim life safety measures.

The plurality of subsystems 114 further includes the asset classifying subsystem 210 that is communicatively connected to the one or more hardware processors 110. The asset classifying subsystem 210 is configured to, in response to determining the plurality of risk levels, classify one or more assets associated with each of the one or more smoke compartments into the plurality of risk levels, based on at least one of one or more asset classes and a location environment associated with each of the one or more assets in each of the one or more smoke compartments comprising the plurality of risk levels. The asset classifying subsystem 210 performs classification by receiving the plurality of risk levels determined for each smoke compartment and then systematically categorizing the one or more assets (fire safety equipment, mechanical systems, electrical components, and building infrastructure) located within those compartments into corresponding risk level categories based on two primary criteria: (a) asset classes (predetermined categories that group assets by function, criticality, and safety importance such as “critical life safety assets,” “fire suppression assets,” “detection assets,” and “support assets”), and (b) the location environment (the specific physical and operational context where each asset is situated, including proximity to high-risk areas, environmental conditions, and compartment characteristics). For example, in a smoke compartment determined to have risk levels of [High Fire Risk, Critical Evacuation Risk, Moderate Equipment Risk], the asset classifying subsystem 210 would classify a fire sprinkler system as “Critical Life Safety Asset” and assign it to the “Critical Evacuation Risk” level due to its essential role in occupant protection, while classifying an HVAC unit in the same compartment as “Support Asset” and assigning it to the “Moderate Equipment Risk” level based on its secondary safety function, and a smoke detector would be classified as “Detection Asset” assigned to “High Fire Risk” level due to its direct fire detection role, with the location environment factors such as the compartment's patient care function, high occupancy density, and proximity to oxygen storage areas influencing these asset classifications to ensure that each asset's risk level assignment accurately reflects both its functional importance and the specific hazards present in its operational environment.

The one or more asset classes are generated using at least one of: asset class inheritance-based AI models and asset class acquisition-based AI models. At least one of: the asset class inheritance-based AI models and asset class acquisition-based AI models is trained to: (a) analyze image data and historical data associated with the assets; and (b) classify the assets into asset classes based on the analyzed image data and historical data. In an embodiment, at least one of the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, Generative Adversarial Networks (GANs), and the like.

The asset classifying subsystem 210 generates asset classes using two specialized AI model types: asset class inheritance-based AI models (which automatically inherit and propagate risk characteristics from parent asset categories to child assets through hierarchical relationships) and asset class acquisition-based AI models (which dynamically learn and acquire new asset classifications by analyzing real-time data and environmental conditions), wherein these AI models are trained to analyze image data (visual information captured from cameras showing asset physical characteristics, installation conditions, and operational status) and historical data (archived records of asset performance, maintenance history, failure patterns, and incident involvement) to automatically classify assets into appropriate asset classes based on learned patterns and relationships. For example, an asset class inheritance-based AI model trained on 10,000 images of fire suppression equipment and 5 years of maintenance records might analyze a new sprinkler head image showing corrosion patterns and historical data indicating 15% failure rate in similar humid environments, then automatically inherit the classification “Critical Life Safety Asset—High Maintenance Risk” from its parent category of “Fire Suppression Systems,” while an asset class acquisition-based AI model analyzing the same sprinkler head's real-time performance data (water pressure readings, activation response times) and environmental sensor data (humidity levels, temperature fluctuations) might dynamically acquire a new classification of “Environmentally Compromised Critical Asset” based on detected performance degradation patterns, thereby enabling the system to automatically generate comprehensive asset classes that reflect both inherited safety characteristics and dynamically acquired risk factors specific to each asset's current condition and operational environment.

The second Convolutional Neural Networks (CNNs) process asset image data through multiple convolutional layers that apply filters to detect visual features such as corrosion patterns, physical damage, installation defects, and equipment conditions, followed by pooling layers that reduce dimensionality while preserving important characteristics, enabling automatic asset classification by learning to recognize visual signatures that distinguish different asset types and condition states. For example, a CNN trained on 50,000 images of fire safety equipment can analyze a photograph of a smoke detector and automatically classify it as “Photoelectric Smoke Detector—Good Condition” by recognizing visual features like the detector's circular shape, LED indicator status, mounting configuration, and absence of visible damage or dust accumulation. The Random Forests operate by constructing multiple decision trees that each analyze different subsets of asset features (such as age, maintenance history, performance metrics, and environmental conditions) and make individual classification predictions, then combine these predictions through majority voting to generate robust asset classifications that are less prone to overfitting. For example, a random forest model with 100 decision trees might analyze an HVAC unit where Tree 1 classifies it as “Critical Asset” based on age >15 years, Tree 2 classifies it as “Moderate Risk Asset” based on recent maintenance, and Tree 3 classifies it as “High Priority Asset” based on location in ICU, with the final classification being “Critical Asset” based on majority vote across all trees.

The Long Short Term Memory (LSTM) networks process sequential time-series data related to asset performance, maintenance records, and operational patterns through specialized memory cells that can retain important information over long time periods while forgetting irrelevant details, enabling classification of the assets based on temporal patterns and trends. For example, an LSTM network analyzing 2 years of fire pump performance data (monthly pressure readings, activation times, maintenance intervals) can classify the pump as “Degrading Critical Asset” by detecting subtle patterns like gradually increasing activation response times (2.1 seconds →2.8 seconds over 18 months) and decreasing pressure consistency that indicate impending failure despite normal current readings. The second Support Vector Machines (SVMs) based models classify assets by finding optimal decision boundaries (hyperplanes) in high-dimensional feature spaces that separate different asset classes, using kernel functions to handle non-linear relationships between asset characteristics such as performance metrics, environmental exposure, and failure history. For example, an SVM model using RBF kernel might classify fire extinguishers by mapping features like [pressure_reading=180_psi, last_inspection=45_days_ago, location_humidity=65%, usage_frequency=low] to a high-dimensional space where it finds the optimal boundary separating “Compliant Assets” from “Non-Compliant Assets,” classifying this extinguisher as “Compliant Asset” based on its position relative to the learned decision boundary. The Generative Adversarial Networks (GANs) consist of two competing neural networks—a generator that creates synthetic asset data and a discriminator that distinguishes between real and synthetic data—which train together to learn complex asset data distributions, enabling classification of the assets by generating realistic asset scenarios and comparing actual assets against learned patterns. For example, a GAN trained on sprinkler system data might generate thousands of synthetic “normal operation” scenarios and “failure condition” scenarios, then classify a real sprinkler system by comparing its current operational parameters (water pressure, valve positions, sensor readings) against the generated patterns to determine whether it matches “normal operation” distribution (classification: “Operational Asset”) or “failure condition” distribution (classification: “At-Risk Asset”).

In an aspect, for classifying the one or more assets associated with each of the one or more smoke compartments into the plurality of risk levels, the asset classifying subsystem 210 is configured to retrieve asset inventory data comprising asset identifiers, asset specifications, installation dates, and maintenance history for each of the one or more assets within each of the one or more smoke compartments from the database. The asset classifying subsystem 210 retrieves asset inventory data by accessing and extracting comprehensive asset information stored in the database, wherein asset identifiers are unique alphanumeric codes or tags that distinguish each individual asset (such as “SPR-ICU-001” for a sprinkler head or “SD-ER-025” for a smoke detector), asset specifications include technical details and performance characteristics (such as flow rate, pressure ratings, detection sensitivity, power requirements, and operational parameters), installation dates record when each asset was originally installed or last replaced (providing age and lifecycle information), and maintenance history contains chronological records of all service activities, inspections, repairs, and performance issues for each asset. For example, when classifying a fire sprinkler system in Smoke Compartment A, the asset classifying subsystem 210 retrieves asset inventory data showing asset identifier “SPR-A-012,” asset specifications including “68° C. activation temperature, 80 GPM flow rate, pendant-type sprinkler head,” installation date “Mar. 15, 2018” (indicating 5.8 years of service), and maintenance history showing “Annual inspection passed (January 2023), pressure test completed (January 2023), head replacement due to corrosion (September 2022), flow test satisfactory (January 2022),” which provides the foundational data necessary for the AI models to analyze the asset's current condition, reliability, and appropriate risk classification within the smoke compartment's risk level framework.

For classifying the one or more assets associated with each of the one or more smoke compartments into the plurality of risk levels, the asset classifying subsystem 210 is configured to determine asset criticality levels by analyzing functional importance of each of the one or more assets to life safety operations within the corresponding one or more smoke compartments using predefined criticality matrices. The asset classifying subsystem 210 determines asset criticality levels by performing systematic analysis that evaluates the functional importance of each asset to life safety operations (essential activities and systems required to protect human life during fire emergencies, including detection, alarm, suppression, evacuation, and emergency communication functions) within their corresponding smoke compartments, utilizing predefined criticality matrices (structured evaluation frameworks that contain predetermined scoring criteria, weighting factors, and classification rules based on regulatory standards, industry best practices, and facility-specific safety requirements) to assign numerical criticality scores and categorical importance levels to each asset. For example, when analyzing a smoke detector in an ICU smoke compartment, the predefined criticality matrix might evaluate functional importance factors such as “detection capability” (score: 9/10 for early fire detection), “life safety impact” (score: 10/10 for patient protection), “regulatory requirement” (score: 10/10 for code compliance), “evacuation support” (score: 8/10 for alarm notification), and “system redundancy” (score: 6/10 for backup detection), resulting in a total criticality score of 43/50 and classification as “Critical Level 1 Asset,” while a general lighting fixture in the same compartment might score “illumination support” (score: 4/10), “life safety impact” (score: 3/10), “regulatory requirement” (score: 2/10), “evacuation support” (score: 5/10), and “system redundancy” (score: 7/10) for a total of 21/50 and classification as “Non-Critical Level 3 Asset,” thereby enabling the subsystem to prioritize assets based on their actual contribution to life safety operations rather than treating all assets equally.

For classifying the one or more assets associated with each of the one or more smoke compartments into the plurality of risk levels, the asset classifying subsystem 210 is configured to map asset locations to specific zones within each of the one or more smoke compartments using coordinate data, floor plans, and spatial relationship algorithms to establish the location environment for each asset. The asset classifying subsystem 210 maps asset locations to specific zones by utilizing coordinate data (precise numerical positioning information such as X, Y, Z coordinates or grid references that pinpoint each asset's exact physical location within the facility), floor plans (architectural drawings and digital blueprints that show the spatial layout, room boundaries, corridors, and structural elements of each smoke compartment), and spatial relationship algorithms (computational methods that calculate distances, proximities, adjacencies, and geometric relationships between assets and environmental features) to establish the location environment for each asset (the specific physical and operational context surrounding each asset, including nearby hazards, accessibility factors, environmental conditions, and functional zone characteristics). For example, when mapping a fire extinguisher with coordinate data showing position (X: 125.3 m, Y: 67.8 m, Floor: 3), the asset classifying subsystem 210 uses floor plans to identify that this location is in “Zone 3B—Patient Care Corridor” within Smoke Compartment A, then applies spatial relationship algorithms to calculate that the extinguisher is 15 meters from the nearest exit, 8 meters from oxygen storage (high-risk area), 3 meters from a nurse station (high-traffic zone), and 12 meters from the nearest sprinkler head, thereby establishing a location environment classification of “High-Access, Moderate-Risk Zone with Critical Proximity to Oxygen Hazard,” which influences how this asset is classified within the smoke compartment's risk level framework and determines appropriate interim life safety measures for its protection and accessibility during emergency situations.

For classifying the one or more assets associated with each of the one or more smoke compartments into the plurality of risk levels, the asset classifying subsystem 210 is configured to apply the asset class inheritance-based AI models to automatically inherit risk characteristics from parent asset categories and propagate risk classifications to child assets based on hierarchical asset relationships. The asset classifying subsystem 210 applies asset class inheritance-based AI models by utilizing hierarchical classification structures where parent asset categories (broad, high-level asset groups such as “Fire Suppression Systems,” “Detection Systems,” or “Emergency Communication Systems”) contain predefined risk characteristics, safety properties, and operational parameters that are automatically inherited by child assets (specific individual assets or sub-categories that belong to the parent category), with the AI models using inheritance algorithms to propagate risk classifications down the hierarchy based on established parent-child relationships, ensuring consistent risk assessment across related assets while allowing for specific modifications based on individual asset conditions. For example, when a parent asset category “Fire Suppression Systems” is classified with risk characteristics including “Critical Life Safety Function=True,” “Failure Impact=High,” “Regulatory Priority=Level 1,” and “Maintenance Frequency=Monthly,” the asset class inheritance-based AI model automatically propagates these characteristics to all child assets including “Sprinkler Head SPR-A-012,” “Fire Pump FP-B-003,” and “Standpipe System SP-C-007,” so that Sprinkler Head SPR-A-012 inherits the parent's “Critical Life Safety Function=True” and “Failure Impact=High” classifications, but the AI model may modify the inherited “Maintenance Frequency” from “Monthly” to “Annual” based on the specific child asset's operational requirements, thereby ensuring that all fire suppression assets maintain consistent critical safety classifications while allowing for asset-specific operational adjustments within the hierarchical framework.

For classifying the one or more assets associated with each of the one or more smoke compartments into the plurality of risk levels, the asset classifying subsystem 210 is configured to execute the asset class acquisition-based AI models to dynamically acquire new risk classifications by analyzing real-time performance data and environmental conditions affecting each of the one or more assets. The asset classifying subsystem 210 executes asset class acquisition-based AI models by performing dynamic analysis that continuously monitors and processes real-time performance data (current operational metrics, sensor readings, and system status information such as pressure levels, temperature readings, activation response times, power consumption, and error codes) and environmental conditions (surrounding physical factors including humidity levels, temperature fluctuations, air quality, vibration levels, corrosive exposure, and operational stress factors) affecting each asset, then uses machine learning algorithms to dynamically acquire new risk classifications (updated categorizations that reflect current asset condition and risk profile) by identifying patterns, anomalies, and performance degradation indicators that may not have been present during initial classification. For example, a fire pump initially classified as “Standard Operational Asset” based on historical data might be continuously monitored by the asset class acquisition-based AI model, which analyzes real-time performance data showing gradually increasing activation response times (2.1 seconds →3.8 seconds over 6 months), decreasing pressure output (180 PSI →165 PSI), and environmental conditions revealing high humidity exposure (85% RH) and excessive vibration levels (0.8 g acceleration), causing the AI model to dynamically acquire a new risk classification of “Degrading Critical Asset—High Maintenance Priority” based on the detected performance decline patterns and adverse environmental impacts, thereby enabling the asset classifying subsystem 210 to proactively identify emerging risks and update asset classifications in real-time rather than relying solely on static historical assessments.

For classifying the one or more assets associated with each of the one or more smoke compartments into the plurality of risk levels, the asset classifying subsystem 210 is configured to correlate the one or more asset classes with the plurality of risk levels associated with each smoke compartment by matching asset safety functions, failure impact potential, and regulatory compliance requirements to the determined plurality of risk levels. The asset classifying subsystem 210 correlates asset classes with smoke compartment risk levels by performing systematic matching analysis that aligns asset safety functions (the specific life safety roles and protective capabilities each asset provides, such as fire detection, suppression, alarm notification, or evacuation support), failure impact potential (the severity and scope of consequences that would occur if the asset fails to operate properly, including immediate danger to occupants, system cascade failures, and emergency response complications), and regulatory compliance requirements (mandatory safety standards, codes, and legal obligations that govern asset performance and maintenance, such as NFPA standards, Joint Commission requirements, and local fire codes) with the determined plurality of risk levels for each smoke compartment, using correlation algorithms that match asset characteristics to appropriate risk categories based on functional alignment and safety criticality. For example, in Smoke Compartment A with determined risk levels of [High Fire Risk, Critical Evacuation Risk, Moderate Equipment Risk], a smoke detector classified as “Detection Asset Class” would be correlated to the “High Fire Risk” level because its asset safety function is fire detection, its failure impact potential includes delayed fire discovery and compromised early warning (high severity), and regulatory compliance requirements mandate immediate detection capability in patient care areas, while a fire sprinkler system classified as “Suppression Asset Class” would be correlated to the “Critical Evacuation Risk” level because its asset safety function is fire suppression to enable safe evacuation, its failure impact potential includes uncontrolled fire spread threatening occupant safety (critical severity), and regulatory compliance requirements specify automatic suppression in healthcare facilities, thereby ensuring that each asset's risk level assignment accurately reflects both the compartment's specific hazards and the asset's role in mitigating those risks.

For classifying the one or more assets associated with each of the one or more smoke compartments into the plurality of risk levels, the asset classifying subsystem 210 is configured to assign weighted risk factors to each of the one or more assets based on proximity to high-risk areas, interdependencies with other critical assets, and potential cascade failure effects within the smoke compartments. The asset classifying subsystem 210 assigns weighted risk factors by performing comprehensive risk assessment that evaluates three critical criteria: proximity to high-risk areas (physical distance and spatial relationship between each asset and locations with elevated fire hazards, such as oxygen storage rooms, electrical panels, chemical storage, or areas with combustible materials), interdependencies with other critical assets (functional relationships and operational connections where one asset's failure directly affects another asset's performance, creating chains of dependency that can amplify overall system risk), and potential cascade failure effects (the likelihood and severity of sequential failures where one asset's malfunction triggers failures in connected or dependent assets, potentially leading to widespread system breakdown), then applies mathematical weighting algorithms that assign numerical risk multipliers to each asset based on these factors. For example, a fire sprinkler head located 5 meters from an oxygen storage room (high-risk area proximity=weight factor 1.8), connected to a fire pump that also serves 12 other sprinkler zones (high interdependency=weight factor 1.5), and whose failure could disable suppression for the entire ICU wing causing potential cascade failure affecting patient evacuation systems and smoke control (high cascade potential=weight factor 2.0) would receive a combined weighted risk factor of 5.4 (1.8×1.5×2.0), while a standard corridor light fixture located 50 meters from any high-risk areas (low proximity=weight factor 1.0), operating independently without critical dependencies (low interdependency=weight factor 1.0), and whose failure would not affect other safety systems (minimal cascade potential=weight factor 1.1) would receive a weighted risk factor of 1.1, thereby ensuring that assets with greater potential to impact overall smoke compartment safety receive appropriately higher risk classifications and priority for interim life safety measures.

For classifying the one or more assets associated with each of the one or more smoke compartments into the plurality of risk levels, the asset classifying subsystem 210 is configured to validate asset classifications by cross-referencing determined asset risk levels against regulatory standards, manufacturer specifications, and historical failure patterns for similar assets. The asset classifying subsystem 210 validates asset classifications by performing comprehensive verification analysis that cross-references the determined asset risk levels (the calculated risk categories and priority levels assigned to each asset based on previous classification steps) against three authoritative benchmarks: regulatory standards (official safety codes, compliance requirements, and legal mandates such as NFPA 25 for fire protection systems, Joint Commission standards for healthcare facilities, and local fire codes that specify minimum safety requirements for different asset types), manufacturer specifications (technical documentation, performance parameters, operational limits, and recommended maintenance intervals provided by equipment manufacturers that define normal operating conditions and failure thresholds), and historical failure patterns for similar assets (documented records and statistical data showing how comparable assets in similar environments have performed, failed, or degraded over time, including failure rates, common failure modes, and performance trends), using correlation algorithms and compliance checking procedures to ensure classification accuracy and regulatory alignment. For example, when validating a fire pump classified as “Critical Asset—High Risk,” the subsystem cross-references this classification against NFPA 25 standards requiring weekly testing and annual flow tests (regulatory validation: compliant), manufacturer specifications showing maximum 4-second activation time and 150 PSI minimum pressure (technical validation: current 3.2-second response and 165 PSI output confirm proper classification), and historical failure patterns from a database of 500 similar fire pumps showing 15% failure rate after 12 years of service (statistical validation: this 8-year-old pump's “High Risk” classification is appropriate given the historical trend), thereby confirming that the asset's risk level determination is supported by regulatory requirements, technical specifications, and empirical evidence from comparable equipment performance.

For classifying the one or more assets associated with each of the one or more smoke compartments into the plurality of risk levels, the asset classifying subsystem 210 is configured to generate asset risk profiles comprising asset class, assigned risk level, location environment factors, and classification confidence scores for each of the one or more assets. The asset classifying subsystem 210 generates asset risk profiles by creating comprehensive documentation records that consolidate four essential components for each asset: asset class (the categorical classification such as “Critical Life Safety Asset,” “Fire Suppression Asset,” or “Detection Asset” determined through AI model analysis), assigned risk level (the specific risk category such as “High Risk,” “Critical Risk,” or “Moderate Risk” correlated to the smoke compartment's risk levels), location environment factors (contextual information about the asset's physical and operational setting including proximity to hazards, environmental conditions, accessibility, and zone characteristics), and classification confidence scores (numerical indicators ranging from 0-100% that represent the reliability and certainty of the classification decision based on data quality, model accuracy, and validation results). For example, the asset classifying subsystem 210 generates an asset risk profile for Fire Sprinkler SPR-ICU-015 showing: Asset Class=“Critical Life Safety Asset—Fire Suppression,” Assigned Risk Level=“High Risk” (correlated to the ICU smoke compartment's High Fire Risk level), Location Environment Factors=“Zone 2A-ICU Patient Care, 8 m from oxygen storage, high humidity environment (75% RH), 24/7 occupied space, 3 m ceiling height, concrete construction,” and Classification Confidence Score=“92%” (based on high-quality sensor data, recent calibration, complete maintenance records, and successful validation against regulatory standards), thereby providing facility managers with a complete risk assessment summary that enables informed decision-making regarding interim life safety measures, maintenance priorities, and emergency response planning for each individual asset within the facility's fire safety system.

For classifying the one or more assets associated with each of the one or more smoke compartments into the plurality of risk levels, the asset classifying subsystem 210 is configured to update asset classifications dynamically in response to changes in smoke compartment risk levels, asset performance degradation, or modifications to the location environment within the smoke compartments. The asset classifying subsystem 210 updates asset classifications dynamically by continuously monitoring three trigger conditions and automatically recalculating asset risk categories when significant changes occur: changes in smoke compartment risk levels (modifications to the overall risk assessment of the compartment from “Moderate Risk” to “High Risk” due to new fire hazards or system failures), asset performance degradation (declining operational effectiveness, increasing failure rates, or deteriorating condition indicators detected through real-time monitoring such as slower response times, reduced output capacity, or increased error frequencies), and modifications to the location environment (alterations to the physical or operational context surrounding the asset including new hazard introductions, occupancy changes, construction modifications, or environmental condition shifts), with the subsystem applying real-time classification algorithms that instantly recalculate asset classes, risk levels, and priority rankings whenever these trigger conditions are detected. For example, a fire sprinkler initially classified as “Standard Operational Asset—Moderate Risk” in a storage room smoke compartment might be dynamically updated to “Critical Asset—High Risk” when the smoke compartment's risk level changes from “Low Risk” to “High Risk” due to introduction of flammable materials storage, combined with asset performance degradation showing 25% reduction in water pressure output over 3 months, and location environment modifications including installation of new electrical equipment creating additional ignition sources within 5 meters of the sprinkler, causing the subsystem to automatically recalculate and update the asset's classification to reflect the elevated risk conditions and ensure that interim life safety measures appropriately address the changed circumstances.

The plurality of subsystems 114 further includes the score assigning subsystem 212 that is communicatively connected to the one or more hardware processors 110. The score assigning subsystem 212 is configured to assign the plurality of risk assessment scores to each of the one or more inspection points corresponding to the classified one or more assets. In an exemplary embodiment, the risk assessment score corresponds to at least one of an importance and a potential harm created when the one or more inspection point fails an inspection. Each of the one or more inspection points corresponds to a requirement of an element of performance (EP). In an exemplary embodiment, the risk assessment is not performed for a failed inspection point when the risk assessment score associated with the failed inspection point is zero. In an exemplary embodiment, the risk assessment for a failed inspection point is delayed for a predetermined time when a “time to resolve” option is selected for a failed inspection point, and wherein risk assessment for the failed inspection point is performed when the failed inspection point is not completed within the predefined time.

The score assigning subsystem 212 performs risk assessment score assignment by systematically evaluating each inspection point (specific checkable elements, components, or requirements associated with each classified asset that must be tested, verified, or assessed during safety inspections, such as sprinkler head water pressure, smoke detector sensitivity, fire door closure mechanism, or emergency lighting battery backup) corresponding to the classified assets within each smoke compartment, then assigns the plurality of risk assessment scores (multiple numerical values that quantify different aspects of risk including importance level, potential harm severity, regulatory compliance impact, and failure consequence magnitude) to each inspection point based on predetermined scoring criteria that consider both the inspection point's criticality to life safety operations and the potential consequences if that specific inspection point fails its required assessment. The plurality of risk assessment scores correspond to at least one of two critical factors: importance (the significance, criticality, and priority level of each inspection point to overall life safety operations, system functionality, and regulatory compliance) and potential harm created when the inspection points fail an inspection (the severity, scope, and magnitude of negative consequences including occupant injury, property damage, system cascade failures, or regulatory violations that would occur if the inspection point does not meet its required performance standard), wherein each inspection point corresponds to a requirement of an element of performance (EP) which are specific, measurable performance standards established by regulatory bodies such as The Joint Commission that define mandatory compliance criteria for life safety systems and equipment.

For example, for a classified fire sprinkler asset in an ICU smoke compartment, the score assigning subsystem identifies multiple inspection points including “water pressure test” (assigned risk assessment scores: importance=95/100, potential harm=90/100, regulatory impact=100/100), “sprinkler head obstruction check” (assigned scores: importance=85/100, potential harm=80/100, regulatory impact=90/100), “pipe corrosion inspection” (assigned scores: importance=70/100, potential harm=75/100, regulatory impact=85/100), and “valve operation test” (assigned scores: importance=80/100, potential harm=85/100, regulatory impact=95/100), thereby creating a comprehensive risk assessment framework where each inspection point receives multiple scores that reflect its specific contribution to overall asset safety performance and enable prioritized maintenance and interim life safety measure implementation.

In an aspect, the score assigning subsystem 212 is further configured to determine at least one of: the one or more failed inspection points correspond to risk assessment assets, the location environment of the one or more assets corresponding to at least one of one or more supplementary facilities and one or more third party vendors, and a proximity to a subsequently discovered risk assessment assets. The score assigning subsystem 212 determines at least one of three specific risk-amplifying conditions by performing comprehensive analysis that systematically evaluates and identifies circumstances that warrant increased risk assessment scores: whether the one or more failed inspection points correspond to risk assessment assets (critical fire safety equipment or systems that have been specifically designated as high-importance assets requiring enhanced monitoring and assessment due to their vital role in life safety protection, such as primary smoke detection systems, main fire suppression equipment, or emergency egress components whose failure would create significant safety hazards), whether the location environment of the one or more assets corresponds to at least one of one or more supplementary facilities (additional buildings, annexes, or connected structures that may not have the same level of fire safety infrastructure as the main facility, such as temporary buildings, storage facilities, or auxiliary structures) and one or more third party vendors (external service providers, contractors, or maintenance companies responsible for fire safety equipment who may have different response times, service levels, or accountability standards compared to internal facility management), and whether there is proximity to a subsequently discovered risk assessment assets (the physical closeness or spatial relationship between current failed inspection points and other critical fire safety assets that were identified as high-risk after the initial assessment, creating potential cascade failure scenarios or compounded risk effects). For example, when analyzing failed inspection point FIP-ICU-025 “smoke detector sensitivity failure” in ICU Smoke Compartment A, the score assigning subsystem 212 determines that this failed inspection point corresponds to risk assessment asset SMK-ICU-022 (designated as critical life safety asset due to protecting 24 vulnerable ICU patients), the location environment corresponds to supplementary facility “Temporary ICU Expansion Wing” (constructed as emergency capacity during pandemic with reduced fire safety infrastructure) and third party vendor “ABC Fire Systems Inc.” (external contractor with 4-hour response time versus internal 1-hour response), and proximity analysis reveals subsequently discovered risk assessment asset SPR-ICU-015 (critical fire sprinkler system) located 15 feet away that was identified as high-risk during follow-up inspection, thereby determining that all three risk-amplifying conditions are present and warrant score increment adjustments to reflect the compounded safety risks created by critical asset failure in a supplementary facility managed by external vendors with additional high-risk assets in close proximity.

The score assigning subsystem 212 is further configured to increment the risk assessment score in response to the determined risk assessment assets impacting the aggregated risk assessment score. The score assigning subsystem 212 increments the risk assessment score by performing systematic score adjustment operations that automatically increase the numerical risk values assigned to specific inspection points when the determined risk assessment assets (the critical fire safety equipment identified as high-importance assets requiring enhanced monitoring) are found to be impacting the aggregated risk assessment score (the cumulative numerical value representing total risk from all failed inspection points within a smoke compartment), where incrementing means adding pre-determined numerical values or percentage increases to the original risk assessment scores to reflect the amplified safety hazards created by the identified risk-amplifying conditions, with these score increases designed to ensure that the aggregated risk assessment score accurately represents the true level of danger posed by failed inspection points involving critical assets, supplementary facilities, third-party vendors, or proximity to other high-risk equipment. For example, when the score assigning subsystem 212 determines that failed inspection point FIP-ICU-025 “smoke detector sensitivity failure” involves risk assessment asset SMK-ICU-022 (critical life safety asset), is located in a supplementary facility managed by a third-party vendor, and is in proximity to subsequently discovered risk assessment asset SPR-ICU-015 (critical sprinkler system), the score assigning subsystem 212 increments the original risk assessment score from 75 points to 135 points by applying: +25 points for “critical risk assessment asset designation” (reflecting the vital importance of the failed smoke detector), +20 points for “supplementary facility location with reduced infrastructure” (reflecting increased vulnerability due to temporary construction), +10 points for “third-party vendor management with extended response time” (reflecting delayed repair capabilities), and +5 points for “proximity to subsequently discovered high-risk asset within 20 feet” (reflecting potential cascade failure risk), thereby increasing the individual inspection point contribution to the aggregated risk assessment score from 75 to 135 points, which elevates the smoke compartment's total aggregated score from 385 to 445 points and ensures that the cumulative risk determination accurately reflects the compounded safety hazards created by multiple risk-amplifying factors affecting this critical life safety asset.

The plurality of subsystems 114 further includes the aggregated score determining subsystem 214 that is communicatively connected to the one or more hardware processors 110. The aggregated score determining subsystem 214 is configured to determine the aggregated score of the plurality of risk assessment scores associated with the plurality of failed inspection points (otherwise referred as one or more failed inspection points) for the one or more assets within each of the one or more smoke compartments. The aggregated score determining subsystem 214 performs comprehensive score aggregation by systematically identifying the one or more failed inspection points (specific inspection checkpoints that did not meet their required performance standards during safety assessments, such as a sprinkler head failing water pressure test, smoke detector failing sensitivity calibration, or fire door failing closure timing requirements) for the classified assets within each smoke compartment, then mathematically combines the plurality of risk assessment scores (multiple numerical values including importance scores and potential harm scores) that are specifically associated with these failed inspection points to determine a single aggregated score that represents the cumulative risk impact of all inspection failures within each smoke compartment. For example, in ICU Smoke Compartment A containing three assets where Asset 1 (fire sprinkler) has two failed inspection points with risk assessment scores of [importance: 90, potential harm: 95] and [importance: 75, potential harm: 80], Asset 2 (smoke detector) has one failed inspection point with scores of [importance: 85, potential harm: 90], and Asset 3 (emergency lighting) has one failed inspection point with scores of [importance: 60, potential harm: 70], the aggregated score determining subsystem 214 would combine all failed inspection point scores using aggregation algorithms such as weighted summation: (90+95)+(75+80)+(85+90)+(60+70)=645 total points, or root mean square calculation: √[(90²+95²+75²+80²+85²+90²+60²+70²)/8]=81.2 average severity, thereby producing a single aggregated score that quantifies the overall risk level created by all inspection failures within that specific smoke compartment and enables threshold-based decision making for implementing interim life safety measures.

For determining the aggregated score of the plurality of risk assessment scores associated with the one or more failed inspection points, the aggregated score determining subsystem 214 is initially configured to identify the one or more failed inspection points by retrieving inspection results data and comparing actual inspection outcomes against required element of performance (EP) standards for each of the one or more inspection points within each of the one or more smoke compartments. The aggregated score determining subsystem 214 identifies the one or more failed inspection points by performing systematic data retrieval and comparison analysis, wherein the aggregated score determining subsystem 214 first retrieves inspection results data (documented records of actual test outcomes, measurements, and assessment findings from completed safety inspections, including numerical readings, pass/fail status, timestamps, and inspector observations for each tested component) from the database 104 or inspection management system, then compares these actual inspection outcomes (the real-world results obtained during physical testing and evaluation of each asset component, such as measured water pressure readings, detected smoke sensitivity levels, recorded door closure times, or observed physical conditions) against required element of performance (EP) standards (predetermined benchmark values, acceptable ranges, and compliance criteria established by regulatory bodies like The Joint Commission, NFPA codes, and facility policies that define the minimum acceptable performance levels for each inspection point) for each inspection point within each smoke compartment, using automated comparison algorithms that flag discrepancies where actual results fall below required standards. For example, when analyzing inspection results for a fire sprinkler system in ICU Smoke Compartment A, the aggregated score determining subsystem 214 retrieves inspection results data showing “water pressure test: 145 PSI measured on Jan. 15, 2024” and “sprinkler head obstruction check: 15% of heads partially blocked on Jan. 15, 2024,” then compares these actual outcomes against EP standards requiring “minimum 150 PSI water pressure” and “0% obstruction tolerance,” identifying two failed inspection points because the measured 145 PSI falls below the required 150 PSI standard and the 15% obstruction exceeds the 0% tolerance requirement, thereby systematically flagging all inspection points where actual performance fails to meet regulatory compliance standards within each smoke compartment.

For determining the aggregated score of the plurality of risk assessment scores associated with the one or more failed inspection points, the aggregated score determining subsystem 214 is further configured to filter the plurality of risk assessment scores to isolate the plurality of risk assessment scores corresponding to the one or more failed inspection points during excluding scores from the one or more inspection points that passed the inspection. The aggregated score determining subsystem 214 filters the plurality of risk assessment scores by performing selective data isolation that systematically separates and extracts only the risk assessment scores (numerical values quantifying importance and potential harm) that are specifically associated with the one or more failed inspection points (inspection checkpoints that did not meet required performance standards) while simultaneously excluding scores from the one or more inspection points that passed the inspection (inspection checkpoints that successfully met or exceeded their required performance standards), using filtering algorithms that create a refined dataset containing only the scores relevant to actual compliance failures rather than including scores from satisfactory inspection results. For example, in an ICU smoke compartment where a fire sprinkler system has five total inspection points with the following results: “water pressure test” (failed— scores: importance 90, potential harm 95), “sprinkler head obstruction check” (failed— scores: importance 75, potential harm 80), “pipe corrosion inspection” (passed—scores: importance 70, potential harm 75), “valve operation test” (passed—scores: importance 80, potential harm 85), and “flow alarm test” (passed—scores: importance 65, potential harm 70), the filtering process would isolate only the scores from the two failed inspection points [90, 95, 75, 80] while excluding the scores from the three passed inspection points [70, 75, 80, 85, 65, 70], thereby creating a filtered dataset of [90, 95, 75, 80] that represents only the risk assessment scores associated with actual inspection failures, ensuring that the subsequent aggregated score calculation reflects only the cumulative risk impact of compliance deficiencies rather than being diluted by scores from satisfactory inspection results.

For determining the aggregated score of the plurality of risk assessment scores associated with the one or more failed inspection points, the aggregated score determining subsystem 214 is further configured to apply one or more aggregation models comprising at least one of: weighted summation, root mean square computations, and maximum value selection, to combine the plurality of risk assessment scores associated with the one or more failed inspection points within each smoke compartment.

The Weighted summation (a mathematical approach that multiplies each risk assessment score by a predetermined weight factor based on the inspection point's criticality, then adds all weighted values together to produce a total score that emphasizes more critical failures) combines the plurality of risk assessment scores by assigning predetermined weight factors to each individual score based on the inspection point's criticality, regulatory importance, or safety impact, then multiplying each risk assessment score by its corresponding weight factor and adding all the weighted values together to produce a single aggregated score that emphasizes more critical inspection failures while maintaining proportional representation of all failed inspection points. For example, with failed inspection point scores of [importance: 90, potential harm: 95, importance: 75, potential harm: 80] and assigned weight factors of [1.5 for critical systems, 1.8 for life safety impact, 1.2 for moderate systems, 1.3 for operational impact], the weighted summation calculation would be (90×1.5)+(95×1.8)+(75×1.2)+(80×1.3)=135+171+90+104=500 total aggregated score.

The root mean square computations (a statistical method that squares each individual risk assessment score, calculates the arithmetic mean of these squared values, then takes the square root of the result to produce an aggregated score that amplifies the impact of higher individual scores while maintaining mathematical balance) combine the plurality of risk assessment scores by squaring each individual score to amplify the mathematical impact of higher values, calculating the arithmetic mean (average) of all squared scores, then taking the square root of this mean to produce an aggregated score that emphasizes the severity of the worst failures while maintaining mathematical balance across all inspection point failures. For example, with the same failed inspection point scores of [90, 95, 75, 80], the root mean square calculation would be √[(90²+95²+75²+80²)+4]=√[(8100+9025+5625+6400)÷4]=√[31150÷4]=<7787.5=88.3 aggregated score.

The maximum value selection (a method that identifies and selects the highest individual risk assessment score among all failed inspection points as the representative aggregated score, emphasizing the most severe single failure rather than cumulative impact) combines the plurality of risk assessment scores by identifying and selecting the single highest individual score among all failed inspection points as the representative aggregated score, effectively using the most severe inspection failure as the determining factor for the entire smoke compartment's risk level rather than considering cumulative or average impacts. For example, with failed inspection point scores of [90, 95, 75, 80], the maximum value selection method would simply identify 95 as the highest score and assign 95 as the aggregated score for the entire smoke compartment, representing the principle that the worst single failure determines the overall risk level regardless of other inspection point performance.

For determining the aggregated score of the plurality of risk assessment scores associated with the one or more failed inspection points, the aggregated score determining subsystem 214 is further configured to incorporate smoke compartment weighting factors based on compartment size, occupancy levels, and critical function designations to adjust the aggregated score computation for each of the one or more smoke compartments. The aggregated score determining subsystem 214 incorporates smoke compartment weighting factors by applying mathematical adjustment multipliers that modify the base aggregated score computation based on three critical compartment characteristics: compartment size (the physical dimensions, square footage, and volume of the smoke compartment which affects fire spread potential, evacuation complexity, and suppression system coverage requirements), occupancy levels (the number of people typically present in the compartment including patients, staff, and visitors, which directly impacts life safety risk and evacuation challenges), and critical function designations (the operational importance and specialized activities performed within the compartment such as intensive care, surgical operations, emergency services, or life support functions that require enhanced protection), with these weighting factors applied as multipliers to increase or decrease the aggregated score based on the compartment's inherent risk characteristics. For example, ICU Smoke Compartment A with a base aggregated score of 85 might have weighting factors applied as follows: compartment size factor of 1.3 (large 2,500 sq ft area requiring extensive suppression coverage), occupancy level factor of 1.8 (high occupancy with 24 patients and 12 staff members including vulnerable patients on life support), and critical function designation factor of 2.0 (intensive care operations requiring maximum life safety protection), resulting in an adjusted aggregated score of 85×1.3×1.8×2.0=398.1, while a small storage room with the same base score of 85 would receive weighting factors of 0.8 (small 200 sq ft size), 0.5 (minimal occupancy with occasional access), and 0.6 (non-critical storage function), producing an adjusted aggregated score of 85×0.8×0.5×0.6=20.4, thereby ensuring that compartments with greater size, higher occupancy, and more critical functions receive appropriately elevated risk scores that reflect their enhanced life safety requirements.

For determining the aggregated score of the plurality of risk assessment scores associated with the one or more failed inspection points, the aggregated score determining subsystem 214 is further configured to determine cumulative risk impact (i.e., combined total effect of multiple failed inspection points within the same smoke compartment that creates greater overall danger than individual failures would cause separately) by analyzing the combined effect of the plurality of failed inspection points within the same smoke compartment, comprising potential synergistic effects amplifying overall risk levels. The aggregated score determining subsystem 214 determines cumulative risk impact by performing comprehensive analysis that evaluates the combined effect of multiple failed inspection points within the same smoke compartment, recognizing that the total risk is not simply the sum of individual failures but includes potential synergistic effects (interactive relationships where the combination of multiple failures creates amplified risk levels that exceed the additive impact of individual failures, such as when one system failure compromises another system's effectiveness or when multiple deficiencies create cascade failure scenarios) that can significantly amplify overall risk levels beyond what would be expected from isolated individual failures. For example, in an ICU smoke compartment where three separate inspection points have failed— a smoke detector with reduced sensitivity (individual risk impact: moderate), a fire sprinkler with low water pressure (individual risk impact: moderate), and an emergency lighting system with failing battery backup (individual risk impact: low)—the cumulative risk impact analysis would identify synergistic effects such as the failed smoke detector delaying fire discovery which allows fires to grow larger before the compromised sprinkler system attempts suppression, while the failed emergency lighting would impede evacuation during the resulting emergency, creating a combined cumulative risk impact rated as “critical” rather than the “moderate” rating that would result from simply adding the three individual moderate/low impacts, because the interaction between these failures creates a scenario where early detection failure leads to suppression system inadequacy during evacuation visibility problems, demonstrating how multiple seemingly manageable individual failures can synergistically amplify to create life-threatening emergency conditions that require immediate interim life safety measures.

For determining the aggregated score of the plurality of risk assessment scores associated with the one or more failed inspection points, the aggregated score determining subsystem 214 is further configured to apply temporal decay functions (mathematical algorithms that systematically reduce the influence or weighting of older data points over time while maintaining or increasing the emphasis on more recent data points, using time-based coefficients) to adjust the plurality of risk assessment scores based on time elapsed as each inspection point failure was identified. The recent inspection point failures receive optimized weighting in the aggregated score determination. The aggregated score determining subsystem 214 applies temporal decay functions by implementing mathematical algorithms that systematically adjust the plurality of risk assessment scores based on the time elapsed since each inspection point failure was identified, where temporal decay functions are time-based mathematical formulas (such as exponential decay, linear decay, or logarithmic decay models) that reduce the weighting or influence of older inspection failures while maintaining or enhancing the impact of more recent failures, recognizing that recent inspection point failures receive optimized weighting (increased mathematical emphasis and higher priority scores) in the aggregated score determination because newer failures represent current system conditions and pose immediate risks, while older failures may have been partially mitigated, addressed through temporary measures, or may reflect outdated system states. For example, in an ICU smoke compartment with four failed inspection points discovered at different times—a fire sprinkler pressure failure identified 90 days ago (original risk score: 85, temporal decay factor: 0.6, adjusted score: 85×0.6=51), a smoke detector sensitivity failure identified 30 days ago (original risk score: 80, temporal decay factor: 0.8, adjusted score: 80×0.8=64), an emergency lighting failure identified 7 days ago (original risk score: 70, temporal decay factor: 0.95, adjusted score: 70×0.95=66.5), and a fire door closure failure identified 2 days ago (original risk score: 75, temporal decay factor: 1.0, adjusted score: 75×1.0=75)—the temporal decay function ensures that the most recent fire door failure receives full weighting while progressively reducing the influence of older failures, resulting in an aggregated score that prioritizes current system deficiencies and reflects the reality that recent failures pose more immediate and actionable risks requiring urgent interim life safety measures.

For determining the aggregated score of the plurality of risk assessment scores associated with the one or more failed inspection points, the aggregated score determining subsystem 214 is further configured to normalize the aggregated scores across distinct smoke compartments to adapt consistent comparison and threshold evaluation regardless of compartment size and a number of the one or more assets contained within each compartment. The aggregated score determining subsystem 214 normalizes the aggregated scores across distinct smoke compartments by applying mathematical standardization techniques that adjust and scale the aggregated scores to enable consistent comparison and threshold evaluation regardless of compartment size (the physical dimensions, square footage, and spatial volume of each smoke compartment which can vary dramatically from small utility rooms to large patient care areas) and the number of assets contained within each compartment (the total count of fire safety equipment, detection devices, suppression systems, and other life safety assets that can range from a few items in simple spaces to dozens of components in complex areas), using normalization algorithms such as min-max scaling, z-score standardization, or proportional adjustment methods that convert raw aggregated scores into standardized values that can be fairly compared across compartments of different scales. For example, ICU Smoke Compartment A with 2,500 square feet containing 25 assets might generate a raw aggregated score of 450 from failed inspection points, while Storage Room Compartment B with 200 square feet containing 3 assets might generate a raw aggregated score of 180, but after normalization using a formula such as normalized_score=(raw_score/(compartment_size_factor×asset_count_factor)), the ICU compartment's normalized score becomes 450/(2.5×2.5)=72, while the storage room's normalized score becomes 180/(0.2×0.3)=3000, revealing that despite the storage room's lower raw score, its normalized score indicates a much higher risk density per unit area and per asset, thereby enabling fair threshold-based comparisons where both compartments can be evaluated against the same predetermined threshold values regardless of their vastly different physical characteristics and asset inventories.

For determining the aggregated score of the plurality of risk assessment scores associated with the one or more failed inspection points, the aggregated score determining subsystem 214 is further configured to validate aggregated score accuracy by cross-referencing computed scores against historical incident data and regulatory risk assessment benchmarks for analogous facility types and smoke compartment configurations. The aggregated score determining subsystem 214 validates aggregated score accuracy by performing comprehensive verification analysis that cross-references the computed aggregated scores (the calculated numerical values representing cumulative risk impact from failed inspection points within each smoke compartment) against two authoritative benchmarks: historical incident data (documented records of actual fire emergencies, safety incidents, equipment failures, and emergency responses that have occurred in similar facilities, including incident severity, response times, casualty reports, property damage assessments, and root cause analyses) and regulatory risk assessment benchmarks (standardized risk evaluation criteria, scoring methodologies, and threshold values established by regulatory bodies such as The Joint Commission, NFPA, CMS, and local fire authorities that define acceptable risk levels and compliance standards for analogous facility types and smoke compartment configurations), using correlation algorithms and statistical analysis to ensure that computed scores accurately reflect real-world risk levels and align with established safety standards. For example, when validating an ICU smoke compartment's computed aggregated score of 385, the aggregated score determining subsystem 214 cross-references this score against historical incident data showing that similar ICU compartments with aggregated scores between 350-400 experienced an average of 2.3 fire-related incidents per year with moderate severity outcomes, and against regulatory benchmarks indicating that Joint Commission standards classify scores above 300 as “high risk requiring immediate corrective action” for intensive care environments, thereby confirming that the computed score of 385 accurately represents a high-risk condition that aligns with both empirical evidence from comparable facilities and established regulatory risk assessment criteria, validating that the mathematical aggregation process produces scores that meaningfully correspond to actual safety risks and compliance requirements.

For determining the aggregated score of the plurality of risk assessment scores associated with the one or more failed inspection points, the aggregated score determining subsystem 214 is further configured to generate aggregated score breakdown reports documenting the individual risk assessment scores, weighting factors, and computation methodologies used to determine a final aggregated score for each smoke compartment. The aggregated score determining subsystem 214 generates aggregated score breakdown reports (detailed documents that show how the final risk score for each smoke compartment was calculated, including individual inspection point scores, weighting factors, and the mathematical methods used to combine them) by creating comprehensive documentation that systematically records and presents all computational elements used in the aggregated score calculation process, where these reports document the individual risk assessment scores (the specific numerical values assigned to each failed inspection point including importance scores and potential harm scores), weighting factors (the mathematical multipliers applied based on compartment characteristics, temporal considerations, and criticality assessments), and computation methodologies (the specific mathematical algorithms, formulas, and calculation steps used to combine scores such as weighted summation, root mean square, or maximum value selection methods) to provide complete transparency and auditability for how the final aggregated score was determined for each smoke compartment. For example, an aggregated score breakdown report for ICU Smoke Compartment A might document: “Individual Risk Assessment Scores: Fire Sprinkler Pressure Test (Importance: 90, Potential Harm: 95), Smoke Detector Sensitivity (Importance: 85, Potential Harm: 90), Emergency Lighting Battery (Importance: 70, Potential Harm: 75); Weighting Factors: Compartment Size Factor: 1.3, Occupancy Level Factor: 1.8, Critical Function Factor: 2.0, Temporal Decay Factors: [1.0, 0.8, 0.6]; Computation Methodology: Weighted Summation Formula=[(90+95)+(85+90)+(70+75)]×1.3×1.8×2.0×temporal_average(0.8)=505×3.744=1,890.72; Final Aggregated Score: 1,891 (rounded),” thereby providing facility managers, inspectors, and regulatory authorities with complete documentation of how each component contributed to the final risk assessment score and enabling verification, audit trails, and informed decision-making regarding interim life safety measures.

The plurality of subsystems 114 further includes the action storing subsystem 216 that is communicatively connected to the one or more hardware processors 110. The action storing subsystem 216 is configured to store information associated with the plurality of Interim Life Safety Measure (ILSM) actions and the plurality of correlating deficiency assets, in the database 104. In an embodiment, each ILSM action defines an action related to protecting the one or more individuals at the facility. In an embodiment, each correlating deficiency asset defines a correlation between a deficiency encountered at the facility and at least one ILSM action. The action storing subsystem 216 performs systematic database storage operations by maintaining and organizing two critical types of information within a centralized database: a plurality of Interim Life Safety Measure (ILSM) actions (comprehensive catalog of specific temporary safety measures, protective procedures, equipment deployments, and operational modifications that can be implemented to protect facility occupants during fire safety system failures, where each ILSM action defines a particular protective action such as “deploy portable smoke detectors,” “implement 24-hour fire watch patrol,” “position portable fire extinguishers,” “establish enhanced evacuation protocols,” or “activate backup communication systems”) and a plurality of correlating deficiency assets (structured database records that establish precise relationships and mappings between specific types of facility deficiencies and the most appropriate ILSM actions to address each deficiency scenario, where each correlating deficiency asset defines a correlation between a particular deficiency encountered at the facility and at least one corresponding ILSM action that effectively mitigates the risk created by that deficiency). For example, the action storing subsystem 216 maintains database records such as ILSM Action ID-001: “Deploy portable photoelectric smoke detectors with 15-minute response capability in affected compartment” (defining the specific protective action), ILSM Action ID-002: “Implement continuous 24-hour fire watch patrol with trained personnel conducting hourly safety rounds” (defining another protective action), and Correlating Deficiency Asset CDA-001: “Smoke Detector Sensitivity Failure (2-5% obscuration range)→Applicable ILSM Actions: [ID-001, ID-002, ID-015]” (defining the correlation between smoke detection deficiencies and multiple applicable interim measures), and Correlating Deficiency Asset CDA-002: “Fire Sprinkler Water Pressure Deficiency (10-25 PSI below minimum)→Applicable ILSM Actions: [ID-003, ID-007, ID-012]” (defining the correlation between sprinkler system deficiencies and corresponding suppression-focused interim measures), thereby creating a comprehensive knowledge base that enables the ILSM determining subsystem 218 to automatically select the most appropriate interim life safety measures based on the specific types of deficiencies identified during facility inspections.

The plurality of subsystems 114 further includes the Interim Life Safety Measure (ILSM) determining subsystem 218 that is communicatively connected to the one or more hardware processors 110. The Interim Life Safety Measure (ILSM) determining subsystem 218 to determine one or more ILSMs, when the aggregated score is greater than the pre-determined threshold value for each of the one or more assets within each of the one or more smoke compartments. The ILSM is the health and safety measure to protect the one or more individuals at the facility. The ILSM determining subsystem 218 performs threshold-based risk assessment by systematically comparing the determined aggregated score (the numerical value representing cumulative risk impact from all failed inspection points within a smoke compartment) against the pre-determined threshold value (a specific numerical benchmark established based on facility type, regulatory requirements, occupancy classification, and safety standards that defines the maximum acceptable risk level before mandatory corrective action is required) for each smoke compartment, and when the aggregated score exceeds this threshold, the ILSM determining subsystem 218 automatically triggers the determination of the plurality of ILSMs (multiple Interim Life Safety Measures, which are temporary health and safety measures, protective actions, procedural modifications, equipment deployments, or operational changes designed to reduce immediate risk and protect individuals at the facility until permanent corrections can be implemented) for each of the assets within the affected smoke compartments. For example, if ICU Smoke Compartment A has a determined aggregated score of 385 and the pre-determined threshold value for intensive care units is set at 300 (based on Joint Commission standards and facility risk tolerance), the ILSM determining subsystem 218 would identify that 385 >300, triggering the determination of multiple ILSMs such as “implement 24-hour fire watch patrol for failed smoke detector coverage,” “deploy portable fire extinguishers near assets with failed sprinkler systems,” “establish enhanced evacuation assistance protocols for patients in areas with failed emergency lighting,” and “activate backup communication systems to compensate for failed alarm notification devices,” thereby ensuring that when cumulative inspection failures create unacceptable risk levels, immediate protective measures are automatically identified and implemented to safeguard facility occupants until permanent repairs restore full fire safety system functionality.

For determining the plurality of ILSMs, the ILSM determining subsystem 218 is configured to select the plurality of ILSMs from the database 104, based on deficiencies associated with the one or more failed inspection points and the plurality of correlating deficiency assets. The ILSM determining subsystem 218 selects the plurality of ILSMs from the database by performing systematic correlation analysis that matches deficiencies associated with the one or more failed inspection points (specific types of compliance failures, performance shortfalls, or system malfunctions identified during safety inspections, such as “smoke detector sensitivity below required threshold,” “sprinkler water pressure insufficient,” or “emergency lighting battery backup failure”) with the plurality of correlating deficiency assets (pre-stored database records that establish relationships between specific types of deficiencies and appropriate ILSM actions, where each correlating deficiency asset defines which interim life safety measures are most effective for addressing particular failure scenarios based on regulatory guidance, best practices, and historical effectiveness data), using database query algorithms that retrieve and match applicable ILSM actions based on deficiency type, asset classification, and compartment characteristics. For example, when the ILSM determining subsystem 218 identifies deficiencies including “smoke detector sensitivity failure in ICU patient room” and “fire sprinkler low water pressure in same compartment,” the ILSM determining subsystem 218 queries the database 104 for correlating deficiency assets that match these specific failure types, retrieving correlating records such as “Deficiency Type: Smoke Detection Failure →Recommended ILSMs: [24-hour fire watch, portable smoke detectors, enhanced staff monitoring]” and “Deficiency Type: Sprinkler Pressure Failure →Recommended ILSMs: [portable fire extinguishers, fire watch patrol, evacuation assistance protocols],” then selects the plurality of ILSMs including “implement 24-hour fire watch patrol,” “deploy portable smoke detection devices,” “position portable fire extinguishers,” and “establish enhanced patient evacuation procedures,” thereby ensuring that the selected interim life safety measures are specifically tailored to address the actual deficiencies identified during inspections rather than applying generic safety measures that may not effectively mitigate the specific risks created by the failed inspection points.

For determining the plurality of ILSMs for each of the one or more assets within each of the one or more smoke compartments, the ILSM determining subsystem 218 is initially configured to compare the aggregated score against the pre-determined threshold value for each of the one or more smoke compartments to identify the one or more smoke compartments requiring the plurality of ILSMs. The ILSM determining subsystem 218 compares the aggregated score against the pre-determined threshold value by performing systematic numerical evaluation that takes the calculated aggregated score (the final numerical value representing cumulative risk impact from all failed inspection points within a specific smoke compartment) and mathematically compares it to the pre-determined threshold value (a specific numerical benchmark established for each type of smoke compartment based on facility classification, occupancy type, regulatory requirements, and acceptable risk levels) to determine whether the aggregated score exceeds, meets, or falls below the threshold, thereby identifying which smoke compartments require immediate implementation of interim life safety measures. For example, the ILSM determining subsystem 218 might evaluate five smoke compartments with the following comparisons: ICU Smoke Compartment A (aggregated score: 385) compared against ICU threshold value of 300 (385 >300=requires ILSMs), Operating Room Compartment B (aggregated score: 275) compared against OR threshold value of 250 (275 >250=requires ILSMs), General Patient Room Compartment C (aggregated score: 180) compared against patient room threshold value of 200 (180<200=no ILSMs required), Storage Room Compartment D (aggregated score: 95) compared against storage threshold value of 150 (95<150=no ILSMs required), and Emergency Department Compartment E (aggregated score: 420) compared against ED threshold value of 350 (420 >350=requires ILSMs), thereby identifying that Compartments A, B, and E require plurality of ILSMs because their aggregated scores exceed their respective pre-determined threshold values, while Compartments C and D do not require ILSMs because their risk levels remain within acceptable limits as defined by their threshold values.

For determining the plurality of ILSMs for each of the one or more assets within each of the one or more smoke compartments, the ILSM determining subsystem 218 is further configured to retrieve threshold configuration parameters from the database 104 comprising pre-determined threshold values specific to distinct facility types, occupancy classifications, and regulatory requirements applicable to each of the one or more smoke compartments. The ILSM determining subsystem 218 retrieves threshold configuration parameters by performing systematic database query operations that extract and obtain pre-determined threshold values (specific numerical benchmarks that define maximum acceptable risk levels before mandatory corrective action is required) that are customized and tailored to distinct facility types (different categories of buildings such as hospitals, nursing homes, ambulatory care centers, or outpatient clinics, each with unique risk profiles and safety requirements), occupancy classifications (standardized building use categories defined by fire codes such as Group 1-2 for hospitals with patients unable to self-evacuate, Group 1-1 for assisted living facilities, or Group B for business occupancies, each requiring different safety thresholds), and regulatory requirements (specific compliance standards, safety codes, and legal mandates established by authorities such as The Joint Commission, CMS Conditions of Participation, NFPA Life Safety Code, and local fire departments that dictate minimum acceptable risk levels) applicable to each individual smoke compartment within the facility. For example, when processing smoke compartments in a multi-use healthcare facility, the subsystem retrieves threshold configuration parameters showing ICU compartments (facility type: acute care hospital, occupancy classification: 1-2 high-dependency, regulatory requirements: Joint Commission LS standards) have a threshold value of 300, while general patient rooms (facility type: acute care hospital, occupancy classification: 1-2 standard care, regulatory requirements: NFPA 101 healthcare occupancy) have a threshold value of 200, outpatient procedure rooms (facility type: ambulatory surgery center, occupancy classification: B business with medical procedures, regulatory requirements: CMS ambulatory standards) have a threshold value of 250, and administrative offices (facility type: healthcare support, occupancy classification: B business, regulatory requirements: standard commercial fire codes) have a threshold value of 400, thereby ensuring that each smoke compartment is evaluated against risk thresholds that accurately reflect its specific operational context, patient vulnerability levels, and applicable regulatory compliance requirements.

For determining the plurality of ILSMs for each of the one or more assets within each of the one or more smoke compartments, the ILSM determining subsystem 218 is further configured to identify triggering assets by analyzing which of the one or more assets within each smoke compartment contributed the one or more failed inspection points that caused the aggregated score to exceed the pre-determined threshold value. The ILSM determining subsystem 218 identifies triggering assets by performing detailed forensic analysis that systematically examines and determines which specific assets within each smoke compartment contributed the one or more failed inspection points that directly caused the aggregated score to exceed the pre-determined threshold value, where triggering assets are the particular fire safety equipment, detection devices, suppression systems, or life safety components whose inspection point failures were mathematically significant enough to push the cumulative risk assessment above the acceptable threshold limit, requiring the ILSM determining subsystem 218 to trace back through the aggregation calculation to isolate which individual asset failures were responsible for crossing the risk threshold and necessitating interim life safety measures. For example, in ICU Smoke Compartment A where the aggregated score of 385 exceeded the threshold value of 300, the subsystem performs triggering asset analysis by examining the contribution of each failed asset: Fire Sprinkler System SPR-ICU-015 contributed failed inspection points with risk scores totaling 185 points, Smoke Detector SMK-ICU-022 contributed failed inspection points with risk scores totaling 175 points, Emergency Lighting EML-ICU-008 contributed failed inspection points with risk scores totaling 145 points, and Fire Door FDR-ICU-003 contributed failed inspection points with risk scores totaling 90 points, then determines that if the Fire Sprinkler System (185 points) and Smoke Detector (175 points) had not failed, the aggregated score would have been 385−185 −175=25, which is well below the 300 threshold, thereby identifying Fire Sprinkler System SPR-ICU-015 and Smoke Detector SMK-ICU-022 as the triggering assets whose specific inspection point failures were the primary contributors that caused the compartment's risk level to exceed acceptable limits and require immediate interim life safety measure implementation.

For determining the plurality of ILSMs for each of the one or more assets within each of the one or more smoke compartments, the ILSM determining subsystem 218 is further configured to correlate the one or more failed inspection points with the plurality of correlating deficiency assets stored in the database 104 to determine relationships between specific deficiencies and applicable ILSM actions. The ILSM determining subsystem 218 correlates the one or more failed inspection points with the plurality of correlating deficiency assets by performing systematic database matching analysis that establishes precise relationships between specific deficiencies (the particular types of compliance failures, performance shortfalls, or system malfunctions identified during safety inspections, such as “smoke detector sensitivity 15% below required threshold,” “sprinkler water pressure 20 PSI insufficient,” or “emergency lighting battery providing only 45 minutes instead of required 90 minutes backup”) and applicable ILSM actions (specific interim life safety measures that are most effective for addressing each particular failure scenario), where correlating deficiency assets are pre-stored database records that function as lookup tables containing mappings between deficiency types, failure characteristics, asset classifications, and recommended corrective measures based on regulatory guidance, industry best practices, and historical effectiveness data. For example, when the subsystem identifies failed inspection points including “smoke detector SMK-ICU-022 sensitivity test failure—detected smoke at 4.2% obscuration instead of required 2.0%” and “fire sprinkler SPR-ICU-015 water pressure test failure—measured 145 PSI instead of required 150 PSI minimum,” it queries the correlating deficiency assets database to find matching records such as “Deficiency Type: Smoke Detection Sensitivity Failure (2-5% range)→Asset Class: Critical Detection →Applicable ILSM Actions: [Deploy portable smoke detectors, Implement 24-hour fire watch, Increase staff monitoring frequency]” and “Deficiency Type: Sprinkler Pressure Deficiency (10-20 PSI below minimum)→Asset Class: Fire Suppression →Applicable ILSM Actions: [Position portable fire extinguishers, Establish fire watch patrol, Reduce compartment occupancy],” thereby determining that the specific smoke detector sensitivity failure correlates to detection-focused ILSMs while the sprinkler pressure failure correlates to suppression-focused ILSMs, enabling the subsystem to select targeted interim measures that directly address the root causes and risk implications of each identified inspection point failure.

For determining the plurality of ILSMs for each of the one or more assets within each of the one or more smoke compartments, the ILSM determining subsystem 218 is further configured to select corresponding ILSM actions from the plurality of ILSM actions stored in the database based on the type of deficiencies, asset classifications, and smoke compartment characteristics associated with the one or more failed inspection points. The ILSM determining subsystem 218 selects corresponding ILSM actions from the plurality of ILSM actions stored in the database 104 by performing multi-criteria matching analysis that systematically evaluates and chooses the most appropriate interim life safety measures based on three key selection criteria: the type of deficiencies (specific categories and characteristics of inspection point failures such as “detection system sensitivity failure,” “suppression system pressure deficiency,” “egress system obstruction,” or “notification system communication breakdown,” each requiring different corrective approaches), asset classifications (the categorized risk levels, functional importance, and operational roles of the failed assets such as “Critical Life Safety Asset,” “Primary Fire Suppression Asset,” “Secondary Detection Asset,” or “Support Infrastructure Asset,” which determine the urgency and scope of required interim measures), and smoke compartment characteristics (the specific attributes, operational context, and risk profile of the compartment including occupancy type, patient vulnerability levels, compartment size, critical functions, and environmental conditions that influence which ILSM actions are feasible and effective). For example, when processing failed inspection points in an ICU smoke compartment where a critical smoke detector (asset classification: Critical Life Safety Asset) has a sensitivity failure (deficiency type: Detection System Performance Deficiency) in a high-occupancy patient care area with vulnerable patients on life support (compartment characteristics: Critical Care, High Vulnerability, 24/7 Occupancy), the ILSM determining subsystem 218 queries the database 104 and selects corresponding ILSM actions including “Deploy portable photoelectric smoke detectors with 15-minute response capability,” “Implement continuous 24-hour fire watch with trained personnel,” and “Establish enhanced patient monitoring protocols with 10-minute safety checks,” while rejecting less suitable options like “Reduce compartment occupancy” (incompatible with critical care operations) or “Install temporary heat detectors” (inadequate for smoke detection requirements), thereby ensuring that selected ILSM actions are precisely matched to address the specific deficiency type, account for the asset's critical classification, and remain operationally feasible within the unique characteristics and constraints of the affected smoke compartment.

For determining the plurality of ILSMs for each of the one or more assets within each of the one or more smoke compartments, the ILSM determining subsystem 218 is further configured to prioritize the plurality of ILSM actions based on severity of risk, regulatory compliance requirements, and potential impact on the one or more individuals within each of the one or more smoke compartments. The ILSM determining subsystem 218 prioritizes the plurality of ILSM actions by performing systematic ranking analysis that evaluates and orders the selected interim life safety measures according to three critical prioritization criteria: severity of risk (the magnitude, immediacy, and potential consequences of the safety hazards created by each failed inspection point, where higher severity failures such as complete smoke detection system failures or total fire suppression system breakdowns receive top priority over lower severity issues like minor equipment maintenance needs), regulatory compliance requirements (the urgency and mandatory nature of compliance obligations established by authorities such as The Joint Commission, CMS, NFPA codes, and local fire departments, where violations that could result in immediate facility closure, patient safety citations, or legal penalties receive higher priority than advisory recommendations), and potential impact on the one or more individuals (the scope, vulnerability, and life safety consequences affecting facility occupants including patients, staff, and visitors, where actions protecting the most vulnerable populations such as ICU patients on life support, surgical patients under anesthesia, or mobility-impaired individuals receive highest priority). For example, in an ICU smoke compartment with multiple selected ILSM actions, the ILSM determining subsystem 218 would prioritize “Deploy portable smoke detectors and implement 24-hour fire watch” as Priority 1 (severity: critical detection failure creating immediate fire discovery risk, regulatory: Joint Commission LS.02.01.20 mandatory compliance, impact: 24 vulnerable ICU patients unable to self-evacuate), “Position portable fire extinguishers near patient beds” as Priority 2 (severity: moderate suppression backup needed, regulatory: NFPA 101 recommended practice, impact: localized patient protection), and “Establish enhanced staff communication protocols” as Priority 3 (severity: low administrative improvement, regulatory: facility policy guideline, impact: operational efficiency for staff), thereby ensuring that the most critical life safety measures addressing the highest risks, most stringent regulatory requirements, and greatest potential harm to vulnerable individuals are implemented first, followed by progressively less critical measures in a systematic priority sequence.

For determining the plurality of ILSMs for each of the one or more assets within each of the one or more smoke compartments, the ILSM determining subsystem 218 is further configured to validate ILSM appropriateness by cross-referencing selected ILSM actions against regulatory standards, facility policies, and best practices for similar deficiency scenarios. The ILSM determining subsystem 218 validates ILSM appropriateness by performing comprehensive verification analysis that systematically cross-references the selected ILSM actions (the specific interim life safety measures chosen to address identified deficiencies) against three authoritative benchmarks: regulatory standards (mandatory compliance requirements, safety codes, and legal obligations established by authorities such as The Joint Commission Life Safety standards, NFPA 101 Life Safety Code, CMS Conditions of Participation, and local fire department regulations that define acceptable interim measures and prohibited actions), facility policies (internal organizational procedures, safety protocols, operational guidelines, and institutional standards established by the healthcare facility that govern emergency response, interim safety measures, staff responsibilities, and patient care continuity during safety system failures), and best practices for similar deficiency scenarios (proven effective approaches, industry-recommended solutions, and evidence-based interim measures documented in professional literature, regulatory guidance documents, and case studies from comparable facilities that have successfully addressed identical or similar inspection point failures), using validation algorithms that verify compatibility, effectiveness, and compliance before final ILSM implementation. For example, when validating the selected ILSM action “implement 24-hour fire watch patrol with trained personnel” for a smoke detector failure in an ICU, the ILSM determining subsystem 218 cross-references this action against regulatory standards confirming that Joint Commission LS.02.01.35 explicitly permits trained fire watch as an acceptable interim measure for detection system failures, against facility policies verifying that Hospital Policy FS-205 establishes fire watch procedures and staff training requirements, and against best practices documentation showing that similar ICU facilities successfully used fire watch patrols during detector failures with average response times of 3.2 minutes and zero fire-related incidents, thereby validating that the selected ILSM action is regulatory compliant, institutionally approved, and empirically effective for the specific deficiency scenario, ensuring that only appropriate, legal, and proven interim life safety measures are implemented to protect facility occupants.

For determining the plurality of ILSMs for each of the one or more assets within each of the one or more smoke compartments, the ILSM determining subsystem 218 is further configured to generate ILSM implementation plans comprising specific actions, required resources, implementation timelines, and responsible parties for each determined ILSM within each affected smoke compartment. The ILSM determining subsystem 218 generates ILSM implementation plans by creating comprehensive operational blueprints that systematically document and organize all necessary components for executing each determined interim life safety measure, where these implementation plans comprise four essential elements: specific actions (detailed step-by-step procedures, tasks, and activities that must be performed to implement each ILSM, including equipment deployment, personnel assignments, procedural modifications, and operational changes), required resources (all necessary materials, equipment, personnel, funding, and support services needed to execute the ILSM including portable devices, trained staff, communication systems, and logistical support), implementation timelines (precise schedules, deadlines, and time-sequenced milestones that specify when each component of the ILSM must be initiated, completed, and maintained, ensuring rapid deployment and sustained effectiveness), and responsible parties (specific individuals, departments, roles, and organizational units assigned accountability for implementing, monitoring, and maintaining each aspect of the ILSM including primary implementers, supervisory oversight, and backup personnel). For example, for the determined ILSM “implement 24-hour fire watch patrol with portable smoke detectors” in ICU Smoke Compartment A, the generated implementation plan would specify: specific actions including “deploy two trained fire safety officers per 12-hour shift, conduct hourly compartment patrols, position portable photoelectric smoke detectors at 15-foot intervals, maintain continuous communication with security command center, and document all patrol activities in fire watch log,” required resources including “4 certified fire watch personnel, 8 portable smoke detectors with battery backup, 2 two-way radios, fire watch patrol checklist forms, and emergency communication protocols,” implementation timeline including “initiate fire watch within 2 hours of ILSM determination, complete detector deployment within 4 hours, maintain continuous coverage for estimated 14-day duration until permanent smoke detector repair completion,” and responsible parties including “Fire Safety Manager (primary implementation oversight), Facilities Director (resource procurement), Nursing Supervisor (operational coordination), and Security Chief (personnel scheduling and backup support),” thereby providing facility management with complete operational guidance for successfully implementing each interim life safety measure.

For determining the plurality of ILSMs for each of the one or more assets within each of the one or more smoke compartments, the ILSM determining subsystem 218 is further configured to determine ILSM effectiveness metrics to estimate risk reduction achieved by implementing each determined ILSM action based on historical performance data and risk mitigation models. The ILSM determining subsystem 218 determines ILSM effectiveness metrics (measurements that show how well each interim life safety measure reduces risk and protects people, based on past performance data and safety improvement calculations) by performing quantitative analysis that systematically determines and estimates the risk reduction achieved by implementing each determined ILSM action, where ILSM effectiveness metrics are numerical measurements and statistical indicators that quantify how much each interim life safety measure reduces the overall risk level within the affected smoke compartment, using two primary data sources: historical performance data (documented records of previous ILSM implementations including actual risk reduction outcomes, incident prevention statistics, response time improvements, and safety performance measurements from similar facilities and comparable deficiency scenarios) and risk mitigation models (mathematical algorithms, statistical frameworks, and predictive models that determine expected risk reduction based on the type of interim measure, deficiency characteristics, compartment attributes, and implementation quality), with these metrics expressed as percentage risk reductions, probability improvements, or numerical score adjustments. For example, when determining effectiveness metrics for the ILSM action “implement 24-hour fire watch patrol with portable smoke detectors” in an ICU smoke compartment with a baseline aggregated risk score of 385, the ILSM determining subsystem 218 analyzes historical performance data showing that similar fire watch implementations in ICU environments achieved an average 65% reduction in fire discovery time and 40% reduction in fire-related incidents, then applies risk mitigation models determining that this specific ILSM action should reduce the compartment's risk score by approximately 180 points (from 385 to 205), representing a 47% risk reduction, while also generating effectiveness metrics including “estimated fire detection improvement: 3.2 minutes average response time,” “projected incident prevention rate: 85% based on historical data,” and “risk score reduction confidence interval: 160-200 points with 90% statistical confidence,” thereby providing facility managers with quantitative evidence of each ILSM's expected safety improvement and enabling data-driven decisions about resource allocation and implementation priorities.

For determining the plurality of ILSMs for each of the one or more assets within each of the one or more smoke compartments, the ILSM determining subsystem 218 is further configured to generate a report associated with ILSM determinations comprising justification for each selected ILSM action, expected duration of implementation, and criteria for ILSM termination when permanent corrections are completed. The ILSM determining subsystem 218 generates a report associated with ILSM determinations by creating comprehensive documentation that systematically records and presents three critical components for each selected interim life safety measure: justification for each selected ILSM action (detailed explanations and rationale that document why each specific interim measure was chosen, including the failed inspection points that triggered the need, the risk analysis that supported the selection, the regulatory requirements that mandate the action, and the evidence-based reasoning that demonstrates the appropriateness and necessity of each ILSM for protecting facility occupants), expected duration of implementation (estimated time periods and projected timelines that specify how long each interim life safety measure will remain in effect, based on factors such as the complexity of permanent repairs, availability of replacement equipment, contractor scheduling, regulatory approval processes, and facility operational constraints), and criteria for ILSM termination when permanent corrections are completed (specific conditions, requirements, and verification standards that must be met before each interim measure can be safely discontinued, including successful completion of permanent repairs, passing of follow-up inspections, regulatory approval of corrections, and confirmation that all systems are restored to full operational compliance). For example, the generated ILSM determination report for ICU Smoke Compartment A might document: “ILSM Action: 24-hour fire watch patrol with portable smoke detectors—Justification: Required due to smoke detector SMK-ICU-022 sensitivity failure (4.2% vs. required 2.0% obscuration) creating critical fire detection gap in high-vulnerability patient care area with 24 ICU patients unable to self-evacuate, mandated by Joint Commission LS.02.01.35 interim measure requirements; Expected Duration: 10-14 business days based on detector replacement part availability and contractor scheduling; Termination Criteria: (1) Installation and commissioning of new smoke detector meeting 2.0% sensitivity specification, (2) Successful completion of sensitivity calibration testing by certified technician, (3) Documentation of passing inspection results, (4) Fire department approval of restored detection system, and (5) 24-hour operational verification period confirming proper system integration,” thereby providing facility management, regulatory inspectors, and safety personnel with complete documentation that supports decision-making, ensures accountability, and establishes clear benchmarks for safely transitioning from interim measures back to permanent fire safety system protection.

The plurality of subsystems 114 further includes the asset controlling subsystem 220 that is communicatively connected to the one or more hardware processors 110. The asset controlling subsystem 220 is configured to configure the information associated with the selected plurality of ILSM actions with the plurality of internet of things (IoT) controllers 116 (configured in each of a plurality of IoT devices) for adapting the plurality of IoT controllers to automatically control the one or more assets within each of the one or more smoke compartments to protect the one or more individuals in the one or more smoke compartments of the facility. In an embodiment, each IoT controller of the plurality of IoT controllers is configured in corresponding one or more assets within each of the one or more smoke compartments. The asset controlling subsystem 220 performs automated system integration by systematically configuring the information associated with the selected plurality of ILSM actions (the specific interim life safety measures determined for implementation, such as “activate emergency ventilation,” “deploy portable suppression systems,” “initiate compartment isolation,” or “enhance detection sensitivity”) with the plurality of Internet of Things (IoT) controllers (networked electronic devices equipped with sensors, processors, and communication capabilities that can receive commands, process instructions, and automatically control connected fire safety equipment and building systems), where this configuration process involves translating human-readable ILSM instructions into machine-executable commands that adapt and program each IoT controller to automatically control the one or more assets (fire safety equipment, detection devices, suppression systems, ventilation controls, door mechanisms, lighting systems, and communication devices) within each smoke compartment to protect facility occupants, with each individual IoT controller strategically configured and installed in corresponding assets throughout the smoke compartments to enable real-time automated responses. For example, when the ILSM determining subsystem 218 selects the interim measure “enhance fire detection and activate emergency ventilation in ICU Smoke Compartment A,” the asset controlling subsystem 220 configures IoT Controller IoT-001 (installed in smoke detector SMK-ICU-022) with commands to “increase detection sensitivity to 1.5% obscuration and transmit alerts every 30 seconds,” configures IoT Controller IoT-002 (installed in HVAC damper DMR-ICU-015) with instructions to “activate smoke evacuation mode and increase exhaust fan speed to 150% capacity,” and configures IoT Controller IoT-003 (installed in emergency lighting EML-ICU-008) with parameters to “maintain continuous illumination and activate directional evacuation indicators,” thereby enabling these IoT controllers to automatically execute the determined ILSM actions without human intervention, continuously monitor system performance, and provide real-time feedback to ensure that the automated asset control effectively protects the 24 ICU patients and medical staff within the smoke compartment.

For configuring the information associated with the selected plurality of ILSM actions with the plurality of IoT controllers 116 for adapting the plurality of IoT controllers 116 to automatically control the one or more assets, the asset controlling subsystem 220 is initially configured to identify IoT controller assignments by mapping each of the plurality of IoT controllers 116 to corresponding one or more assets within each of the one or more smoke compartments based on asset location data and controller communication capabilities. The asset controlling subsystem 220 identifies IoT controller assignments (i.e., assigning of the IoT controller) by performing systematic mapping analysis that establishes precise one-to-one or one-to-many relationships between each individual IoT controller 116 and its corresponding assets within specific smoke compartments, where this mapping process utilizes two critical data sources: asset location data (detailed spatial information including GPS coordinates, floor plans, room numbers, zone designations, and physical positioning data that precisely identifies where each fire safety asset is installed within the facility's smoke compartments) and controller communication capabilities (technical specifications defining each IoT controller's wireless range, network protocols, data transmission capacity, and connectivity limitations that determine which assets each controller can effectively monitor and control), using spatial analysis algorithms and communication range calculations to ensure optimal controller-to-asset assignments that maximize coverage while maintaining reliable connectivity. For example, in ICU Smoke Compartment A measuring 2,500 square feet, the asset controlling subsystem 220 identifies IoT controller assignments by mapping IoT Controller IoT-001 (located at coordinates X:125, Y:200 with 50-foot wireless range and Zigbee protocol capability) to corresponding assets including Smoke Detector SMK-ICU-022 (coordinates X:130, Y:195, distance: 7 feet, compatible Zigbee interface), Fire Sprinkler SPR-ICU-015 (coordinates X:140, Y:210, distance: 18 feet, compatible wireless sensor), and Emergency Lighting EML-ICU-008 (coordinates X:115, Y:185, distance: 22 feet, compatible control interface), while mapping IoT Controller IoT-002 (located at coordinates X:200, Y:150 with 75-foot range and WiFi capability) to corresponding assets including HVAC Damper DMR-ICU-025 (coordinates X:220, Y:160, distance: 25 feet, WiFi-enabled actuator) and Fire Door FDR-ICU-003 (coordinates X:180, Y:140, distance: 28 feet, WiFi door controller), thereby ensuring that each IoT controller is assigned to monitor and control only those assets within its effective communication range and technical compatibility, creating a reliable automated control network that can execute ILSM actions across all critical fire safety equipment within the smoke compartment.

For configuring the information associated with the selected plurality of ILSM actions with the plurality of IoT controllers 116 for adapting the plurality of IoT controllers 116 to automatically control the one or more assets, the asset controlling subsystem 220 is further configured to translate the plurality of ILSM actions into control commands by converting the selected plurality of ILSM actions into machine-readable instructions and control parameters compatible with the plurality of IoT controllers 116. The asset controlling subsystem 220 translates the plurality of ILSM actions into control commands (specific digital instructions sent to IoT controllers that tell fire safety equipment exactly what actions to perform, such as “activate sprinkler system” or “increase smoke detector sensitivity to 1.5%) by performing systematic conversion analysis that transforms human-readable interim life safety measures (descriptive instructions such as “implement 24-hour fire watch,” “enhance smoke detection sensitivity,” “activate emergency ventilation,” or “deploy portable suppression systems”) into machine-readable instructions and control parameters (specific digital commands, numerical values, protocol messages, and executable code that the IoT controllers 116 can process and execute, including device activation codes, sensor threshold adjustments, timing parameters, communication protocols, and automated response sequences) that are fully compatible with the plurality of IoT controllers' 116 technical specifications, communication protocols, and operational capabilities, using translation algorithms that map each ILSM action to corresponding device commands while ensuring proper syntax, data formatting, and protocol compliance. For example, when translating the ILSM action “enhance fire detection and activate emergency ventilation in ICU Smoke Compartment A,” the asset controlling subsystem 220 converts this into machine-readable instructions such as: for IoT Controller IoT-001 (smoke detector controller): “SET_SENSITIVITY_THRESHOLD=1.5%, ALERT_INTERVAL=30_SECONDS, ENABLE_CONTINUOUS_MONITORING=TRUE” (adjusting detection parameters), for IoT Controller IoT-002 (HVAC controller): “ACTIVATE_SMOKE_EVACUATION_MODE=ON, SET_EXHAUST_FAN_SPEED=150%, OPEN_DAMPERS=FULL_POSITION, ENABLE_EMERGENCY_VENTILATION=TRUE” (controlling ventilation systems), and for IoT Controller IoT-003 (lighting controller): “EMERGENCY_LIGHTING=CONTINUOUS_ON, ACTIVATE_EVACUATION_INDICATORS=TRUE, BRIGHTNESS_LEVEL=100%” (managing emergency lighting), thereby ensuring that each IoT controller receives precise, executable commands that automatically implement the determined interim life safety measures without requiring human interpretation or manual intervention, enabling seamless automated protection of facility occupants through coordinated IoT device control.

For configuring the information associated with the selected plurality of ILSM actions with the plurality of IoT controllers 116 for adapting the plurality of IoT controllers 116 to automatically control the one or more assets, the asset controlling subsystem 220 is further configured to establish communication protocols between the asset controlling subsystem 220 and the plurality of IoT controllers 116 using at least one of: wireless communication, wired networks, and mesh networking topologies to enable real-time command transmission. The asset controlling subsystem 220 establishes communication protocols (standardized methods and rules that allow the asset controlling subsystem and IoT controllers to send and receive messages, data, and commands through wireless, wired, or network connections) by implementing standardized data transmission frameworks and networking architectures that create reliable, secure, and efficient communication channels between the central asset controlling subsystem 220 and the plurality of distributed IoT controllers 116 throughout the facility, where communication protocols are technical specifications and rules that govern how data is formatted, transmitted, received, and processed between networked devices, utilizing three primary networking approaches: wireless communication (radio frequency technologies such as WiFi, Zigbee, Bluetooth, or cellular networks that transmit commands and data through electromagnetic waves without physical cables), wired networks (physical cable connections such as Ethernet, fiber optic, or serial communication lines that provide stable, high-bandwidth data transmission through direct electrical or optical connections), and mesh networking topologies (distributed network architectures where multiple IoT controllers can communicate with each other and relay messages through interconnected nodes, creating redundant communication pathways that maintain connectivity even if individual devices fail), with these protocols specifically designed to enable real-time command transmission (immediate, low-latency delivery of control instructions and status updates that allow the asset controlling subsystem 220 to send ILSM commands and receive operational feedback within milliseconds or seconds rather than minutes). For example, in ICU Smoke Compartment A, the asset controlling subsystem 220 establishes communication protocols by configuring WiFi wireless communication (802.11 n protocol at 2.4 GHz frequency) to transmit real-time commands to IoT Controller IoT-001 controlling the smoke detection system, implementing wired Ethernet network connections (TCP/IP protocol over Cat6 cables) to ensure reliable communication with IoT Controller IoT-002 managing critical HVAC ventilation systems, and deploying Zigbee mesh networking topology (IEEE 802.15.4 protocol) that allows IoT Controllers IoT-003, IoT-004, and IoT-005 controlling emergency lighting, fire doors, and communication systems to relay messages through each other, thereby creating a robust multi-protocol communication infrastructure that ensures the asset controlling subsystem 220 can instantly transmit ILSM activation commands like “ACTIVATE_EMERGENCY_VENTILATION=TRUE” and immediately receive confirmation responses like “VENTILATION_SYSTEM_ACTIVATED=SUCCESS” within 2-3 seconds, enabling real-time automated protection of facility occupants.

For configuring the information associated with the selected plurality of ILSM actions with the plurality of IoT controllers 116 for adapting the plurality of IoT controllers 116 to automatically control the one or more assets, the asset controlling subsystem 220 is further configured to configure controller operating parameters by programming each IoT controller 116 with specific control logic, safety thresholds, and automated response sequences corresponding to the determined ILSM actions for protecting the one or more individuals. The asset controlling subsystem 220 configures controller operating parameters (specific settings, values, and configurations that control how IoT controllers and fire safety equipment function, such as detection sensitivity levels, response times, and activation thresholds) by performing comprehensive programming operations that systematically install and customize three critical operational components within each individual IoT controller: specific control logic (algorithmic decision-making rules, conditional statements, and programmed instructions that define how each controller should analyze sensor data, evaluate conditions, and determine appropriate responses, such as “IF smoke_density >threshold THEN activate_suppression AND send_alert”), safety thresholds (precise numerical limits, boundary values, and trigger points that define when automated safety actions must be initiated, including temperature limits, smoke density percentages, pressure readings, or time intervals that serve as decision criteria for emergency responses), and automated response sequences (predetermined step-by-step action protocols that specify the exact sequence, timing, and coordination of safety measures that each controller must execute when triggered conditions are detected, ensuring consistent and immediate protective responses without human intervention), where these parameters are specifically tailored and programmed to correspond directly to the determined ILSM actions for protecting facility occupants. The controller operating parameters are specific control logic such as conditional algorithms like “IF smoke_density >2.0% THEN activate_sprinkler_system AND send_alert_to_command_center” or “IF temperature_rise >20° F._per_minute THEN trigger_evacuation_alarm AND open_smoke_dampers,” safety thresholds such as numerical trigger points like “smoke_detection_threshold=1.5% obscuration,” “temperature_alarm_threshold=140° F.,” “sprinkler_activation_pressure=165° F.,” “emergency_lighting_activation_time=10_seconds,” or “fire_door_closure_delay=30_seconds,” and automated response sequences such as step-by-step protocols like “Sequence A: Step 1—Activate smoke detector alarm (0-5 seconds), Step 2—Send notification to fire panel (5-10 seconds), Step 3—Trigger sprinkler pre-action (10-15 seconds), Step 4—Activate emergency ventilation (15-20 seconds), Step 5—Close fire doors (20-25 seconds)” or “Sequence B: Emergency Lighting Protocol—Step 1—Switch to battery backup power, Step 2—Increase illumination to 100% brightness, Step 3—Activate directional evacuation indicators, Step 4—Maintain continuous operation for minimum 90 minutes.”

For example, when configuring IoT Controller IoT-001 installed in the smoke detection system for ICU Smoke Compartment A to implement the determined ILSM action “enhanced fire detection with immediate suppression activation,” the asset controlling subsystem 220 programs specific control logic including “IF (smoke_density >=1.5% obscuration OR temperature_rise >=15° F._per_minute) THEN execute_emergency_protocol,” safety thresholds including “smoke_detection_threshold=1.5% obscuration, temperature_alarm_threshold=135° F., response_time_limit=30_seconds,” and automated response sequences including “Step 1: Activate local alarm within 5 seconds, Step 2: Send alert to fire command center within 10 seconds, Step 3: Trigger sprinkler pre-action system within 15 seconds, Step 4: Activate emergency ventilation within 20 seconds, Step 5: Initiate evacuation lighting within 25 seconds,” thereby ensuring that this IoT controller 116 automatically executes the complete ILSM protection sequence whenever detection thresholds are exceeded, providing immediate automated protection for the 24 vulnerable ICU patients without requiring human decision-making or manual intervention during emergency conditions.

For configuring the information associated with the selected plurality of ILSM actions with the plurality of IoT controllers 116 for adapting the plurality of IoT controllers 116 to automatically control the one or more assets, the asset controlling subsystem 220 is further configured to execute fail-safe mechanisms within each of the plurality of IoT controllers 116 to determine whether safe operation and automatic reversion to safe states when communication failures and system malfunctions occur. The asset controlling subsystem 220 executes fail-safe mechanisms (automatic safety features built into IoT controllers that ensure fire safety equipment switches to a safe operating mode or shuts down safely when communication problems or system malfunctions occur) by implementing built-in safety protocols and automated protective systems within each individual IoT controller that continuously monitor system health and automatically trigger predetermined safe operating modes when communication failures (loss of network connectivity, interrupted data transmission, or inability to receive commands from the central asset controlling subsystem 220) and system malfunctions (hardware failures, software errors, sensor defects, or power supply disruptions) are detected, where fail-safe mechanisms are engineered safety features designed to ensure that the IoT controllers 116 default to the safest possible operational state rather than creating additional hazards when normal operation is compromised, and automatic reversion to safe states means that the IoT controllers 116 immediately switch to pre-programmed emergency operating modes that prioritize occupant protection over normal functionality. For example, when IoT Controller IoT-001 managing the fire suppression system in ICU Smoke Compartment A detects a communication failure (no command signals received from the asset controlling subsystem 220 for 60 seconds), the fail-safe mechanism automatically executes safe state protocols including “revert to local autonomous operation mode,” “maintain sprinkler system in ready-to-activate status,” “continue monitoring smoke detection sensors using last known threshold settings (1.5% obscuration),” “activate local alarm systems if smoke is detected,” and “send emergency beacon signal every 30 seconds to alert maintenance personnel of communication loss,” while if the same IoT controller experiences a system malfunction such as sensor calibration error, the fail-safe mechanism triggers automatic reversion including “switch to backup sensor array,” “reduce detection threshold to most sensitive setting (1.0% obscuration) for maximum protection,” “activate continuous monitoring mode,” and “maintain all safety systems in heightened alert status,” thereby ensuring that even when IoT controllers lose communication with the central system or experience technical failures, they continue protecting facility occupants through autonomous safe operation rather than becoming safety hazards themselves.

For configuring the information associated with the selected plurality of ILSM actions with the plurality of IoT controllers 116 for adapting the plurality of IoT controllers 116 to automatically control the one or more assets, the asset controlling subsystem 220 is further configured to coordinate multi-controller operations by synchronizing actions between the plurality of IoT controllers 116 when ILSM implementation requires coordinated control of interdependent assets across one or more areas of the smoke compartments. The asset controlling subsystem 220 coordinates multi-controller operations (coordinated actions where multiple IoT controllers work together simultaneously to control different fire safety equipment in a synchronized manner to achieve a common safety objective) by performing synchronized orchestration analysis that systematically manages and synchronizes actions between multiple IoT controllers 116 when ILSM implementation requires coordinated control of interdependent assets (fire safety equipment, detection devices, suppression systems, ventilation controls, and emergency systems that must work together in precise timing and sequence to achieve effective life safety protection) across one or more areas of the smoke compartments, where coordinated control means that multiple IoT controllers 116 must execute their individual commands simultaneously or in carefully timed sequences to ensure that interconnected safety systems operate as an integrated protective network rather than independent isolated devices, using synchronization protocols that include timing coordination, command sequencing, status verification, and feedback loops to ensure all controllers execute their assigned ILSM actions in proper coordination. For example, when implementing the ILSM action “activate comprehensive fire suppression and evacuation protocol” across ICU Smoke Compartment A, the asset controlling subsystem 220 coordinates multi-controller operations by synchronizing IoT Controller IoT-001 (smoke detection system) to “activate detection alarm at T=0 seconds and send trigger signal to other controllers,” IoT Controller IoT-002 (HVAC ventilation system) to “receive trigger signal and activate smoke evacuation mode at T=5 seconds,” IoT Controller IoT-003 (fire suppression system) to “receive coordination signal and initiate sprinkler pre-action sequence at T=10 seconds,” IoT Controller IoT-004 (emergency lighting system) to “activate full illumination and evacuation indicators at T=15 seconds,” and IoT Controller IoT-005 (fire door system) to “begin controlled closure sequence at T=20 seconds to contain smoke while maintaining egress paths,” thereby ensuring that all interdependent assets work together in coordinated timing where smoke detection triggers ventilation activation, which coordinates with suppression system preparation, which synchronizes with emergency lighting activation, which coordinates with fire door management, creating a comprehensive automated life safety response that protects facility occupants through precisely orchestrated multi-controller coordination rather than uncoordinated individual device actions.

For configuring the information associated with the selected plurality of ILSM actions with the plurality of IoT controllers 116 for adapting the plurality of IoT controllers 116 to automatically control the one or more assets, the asset controlling subsystem 220 is further configured to monitor a status of the plurality of IoT controllers by continuously receiving operational feedback, error reports, and performance data from each of the plurality of IoT controllers to verify proper ILSM action execution. The asset controlling subsystem 220 monitors the status of the plurality of IoT controllers 116 by performing continuous surveillance operations that systematically collect, analyze, and evaluate real-time information from each individual IoT controller 116 throughout the facility, where monitoring status means maintaining constant awareness of each controller's operational condition, performance metrics, and execution progress through three primary data streams: operational feedback (real-time status reports, confirmation messages, and performance indicators that each IoT controller 116 transmits back to the central system to confirm successful command execution, current operating parameters, and system health, such as “sprinkler system armed and ready,” “smoke detector sensitivity set to 1.5%,” or “emergency lighting at 100% brightness”), error reports (automated diagnostic messages, fault notifications, and malfunction alerts that controllers generate when they detect problems, failures, or abnormal conditions, including “sensor calibration error detected,” “communication timeout occurred,” or “battery backup power low”), and performance data (quantitative measurements, operational metrics, and effectiveness indicators that document how well each IoT controller is executing its assigned ILSM actions, including response times, accuracy measurements, and system efficiency statistics), with this continuous monitoring specifically designed to verify proper ILSM action execution and ensure that all interim life safety measures are functioning as intended. For example, when monitoring IoT Controller IoT-001 implementing the ILSM action “enhanced smoke detection with 1.5% sensitivity threshold” in ICU Smoke Compartment A, the asset controlling subsystem 220 continuously receives operational feedback including “smoke detector operational status: ACTIVE, current sensitivity setting: 1.5% obscuration, last calibration: successful, detection range: 95% coverage,” error reports such as “ERROR: Sensor drift detected at 14:23:15—automatic recalibration initiated” or “WARNING: Communication latency increased to 2.3 seconds—backup communication protocol activated,” and performance data including “detection response time: average 12 seconds, false alarm rate: 0.2% over 24-hour period, system uptime: 99.8%,” thereby enabling the asset controlling subsystem 220 to verify that the IoT controller 116 is properly executing the determined ILSM action, immediately identify any problems that could compromise occupant protection, and take corrective action to maintain effective interim life safety measure implementation.

For configuring the information associated with the selected plurality of ILSM actions with the plurality of IoT controllers 116 for adapting the plurality of IoT controllers 116 to automatically control the one or more assets, the asset controlling subsystem 220 is further configured to validate control effectiveness by analyzing the real-time sensor data and asset performance metrics to determine that the automatically controlled assets are successfully protecting the one or more individuals as intended by the plurality of ILSM actions. The asset controlling subsystem 220 validates control effectiveness (how well the automated fire safety equipment is actually working to protect people as intended by the interim life safety measures) by performing comprehensive verification analysis that systematically examines and evaluates two critical data sources: real-time sensor data (continuous measurements and readings from environmental sensors, detection devices, and monitoring equipment throughout the smoke compartments, including current smoke density levels, temperature readings, humidity measurements, air quality indicators, occupancy detection data, and system performance metrics that provide objective evidence of actual conditions within the protected areas) and asset performance metrics (quantitative measurements and operational statistics that document how well each controlled asset is functioning, including response times, accuracy rates, system uptime, detection sensitivity levels, suppression system pressure readings, ventilation flow rates, and emergency lighting illumination levels), using analytical algorithms and comparison models to determine that the automatically controlled assets are successfully protecting the one or more individuals as intended by the plurality of ILSM actions (confirming that the interim life safety measures are achieving their designed protective objectives and effectively reducing risk to facility occupants). For example, when validating the control effectiveness of the ILSM action “enhanced smoke detection and emergency ventilation activation” in ICU Smoke Compartment A, the asset controlling subsystem 220 analyzes real-time sensor data showing “current smoke density: 0.8% obscuration (well below 1.5% alarm threshold), temperature: 72° F. (normal range), air quality index: 95% (excellent), occupancy sensors: 24 patients and 6 staff detected and monitored” and asset performance metrics including “smoke detector response time: 8 seconds average (exceeds 15-second target), ventilation system air exchange rate: 12 changes per hour (exceeds 8 required), emergency lighting: 100% operational at full brightness, fire door systems: 98% operational status,” then determines that these measurements confirm the automatically controlled assets are successfully protecting the 30 individuals in the compartment because smoke levels remain well below dangerous thresholds, air quality is maintained at safe levels, all occupants are continuously monitored, detection systems are responding faster than required, ventilation is providing adequate air exchange, and emergency systems are fully operational, thereby validating that the ILSM implementation is effectively achieving its intended protective objectives and ensuring occupant safety as designed.

For configuring the information associated with the selected plurality of ILSM actions with the plurality of IoT controllers 116 for adapting the plurality of IoT controllers 116 to automatically control the one or more assets, the asset controlling subsystem 220 is further configured to generate control audit logs documenting a plurality of commands sent to the plurality of IoT controllers 116, controller responses, and asset control actions performed for regulatory compliance and system troubleshooting purposes. The asset controlling subsystem 220 generates the control audit logs (detailed records that document all commands sent to IoT controllers, their responses, and the actions performed by fire safety equipment for regulatory compliance and troubleshooting purposes) by performing comprehensive documentation operations that systematically create detailed electronic records capturing three critical categories of automated control activities: a plurality of commands sent to the plurality of IoT controllers 116 (complete chronological records of every instruction, parameter setting, activation signal, and control message transmitted from the central asset controlling subsystem 220 to individual IoT controllers, including command timestamps, target controller identifications, specific instruction content, and transmission confirmation status), controller responses (detailed logs of acknowledgment messages, status confirmations, error notifications, and feedback data that each IoT controller 116 sends back to the central system in response to received commands, documenting successful execution confirmations, failure notifications, and operational status updates), and asset control actions performed (comprehensive records of actual physical actions, system changes, and safety measure implementations executed by controlled assets, including device activations, parameter adjustments, system state changes, and protective measure deployments), where these audit logs serve dual purposes for regulatory compliance (providing documented evidence of proper ILSM implementation, system operation, and safety measure execution required by authorities such as The Joint Commission, CMS, NFPA standards, and local fire departments for inspection verification and compliance demonstration) and system troubleshooting purposes (enabling technical analysis, problem diagnosis, performance evaluation, and system optimization by providing detailed operational history for identifying patterns, diagnosing failures, and improving system performance). For example, the control audit log for ICU Smoke Compartment A implementing the ILSM action “enhanced fire detection and emergency ventilation” would document entries such as “2024-01-15 14:23:12—COMMAND SENT: Controller IoT-001, SET_SMOKE_SENSITIVITY=1.5%, ENABLE_CONTINUOUS_MONITORING=TRUE,” “2024-01-15 14:23:15—CONTROLLER RESPONSE: IoT-001, COMMAND_ACKNOWLEDGED, SENSITIVITY_UPDATED_SUCCESSFULLY, STATUS=OPERATIONAL,” “2024-01-15 14:23:18—ASSET CONTROL ACTION: Smoke Detector SMK-ICU-022, Sensitivity adjusted from 2.0% to 1.5% obscuration, Detection range verified at 95% coverage,” “2024-01-15 14:23:45—COMMAND SENT: Controller IoT-002, ACTIVATE_EMERGENCY_VENTILATION=TRUE, SET_EXHAUST_RATE=150%,” “2024-01-15 14:23:48—CONTROLLER RESPONSE: IoT-002, VENTILATION_ACTIVATED, EXHAUST_RATE_INCREASED, AIRFLOW_CONFIRMED,” and “2024-01-15 14:23:52—ASSET CONTROL ACTION: HVAC System HVC-ICU-025, Emergency ventilation mode activated, Air exchange rate increased to 12 changes/hour,” thereby creating a complete audit trail that demonstrates regulatory compliance with interim life safety measure requirements while providing technical personnel with detailed operational data for system maintenance, performance analysis, and troubleshooting when issues arise.

For configuring the information associated with the selected plurality of ILSM actions with the plurality of IoT controllers 116 for adapting the plurality of IoT controllers 116 to automatically control the one or more assets, the asset controlling subsystem 220 is further configured to update one or more configurations of the plurality of IoT controllers 116 dynamically in response to changes in ILSM requirements, asset status modifications, and emergency conditions that require immediate adjustment of automated control parameters. The asset controlling subsystem 220 updates one or more configurations of the plurality of IoT controllers 116 dynamically by performing real-time reconfiguration operations that automatically modify, adjust, and reprogram IoT controller settings, parameters, and operational instructions in response to three types of triggering events: changes in ILSM requirements (modifications to interim life safety measure specifications such as updated regulatory standards, revised facility policies, enhanced safety protocols, or new risk assessments that necessitate different protective measures, including changes like “increase smoke detection sensitivity from 1.5% to 1.0% obscuration” or “extend fire watch patrol frequency from hourly to every 30 minutes”), asset status modifications (alterations in the operational condition, performance capability, or functional status of controlled fire safety equipment such as equipment repairs, component replacements, system upgrades, or performance degradation that require corresponding controller adjustments, including scenarios like “smoke detector recalibrated to new sensitivity range” or “sprinkler system pressure increased after pump repair”), and emergency conditions (immediate life-threatening situations, critical system failures, or urgent safety incidents that demand instant modification of automated control parameters to provide maximum protection, such as “active fire detected requiring immediate suppression activation” or “evacuation ordered requiring emergency lighting and door control override”), where dynamic updating means these configuration changes occur automatically and immediately without human intervention or system downtime. For example, when an active fire is detected in ICU Smoke Compartment A at 15:45:30, the subsystem dynamically updates IoT Controller IoT-001 configuration from normal ILSM settings “smoke\_detection\_threshold=1.5%, alarm\_delay=30\_seconds, suppression\_activation=manual” to emergency response settings “smoke\_detection\_threshold=1.0%, alarm\_delay=0\_seconds, suppression\_activation=automatic\_immediate,” simultaneously updates IoT Controller IoT-002 from standard ventilation parameters “air\_exchange\_rate=8\_changes\_per\_hour, exhaust\_mode=normal” to emergency evacuation parameters “air\_exchange\_rate=15\_changes\_per\_hour, exhaust\_mode=smoke\_evacuation\_maximum,” and updates IoT Controller IoT-003 from routine lighting configuration “emergency\_lighting=standby\_mode, brightness=75%” to full emergency configuration “emergency\_lighting=continuous\_maximum, brightness=100%, evacuation\_indicators=flashing\_directional,” thereby ensuring that all IoT controllers automatically adapt their operational parameters within seconds to provide maximum protection during the evolving emergency situation without requiring manual intervention or system reconfiguration delays.

In an aspect, the plurality of subsystems 114 further includes a work order generating subsystem (not shown in FIG. 2), that is communicatively connected to the one or more hardware processors. The work order generating subsystem is configured to generate at least one of: the one or more maintenance work orders and the one or more corrective work orders, based on the determined one or more ILSMs. The work order generating subsystem performs automated documentation operations by systematically creating formal work requests and task assignments based on the determined plurality of ILSMs (the specific interim life safety measures that have been identified and selected for implementation), where this work order generating subsystem generates two distinct types of work orders: one or more maintenance work orders (formal requests for scheduled, preventive, or routine maintenance activities that support the ongoing implementation and effectiveness of interim life safety measures, including tasks such as equipment inspections, system calibrations, component replacements, or performance verifications that ensure ILSM-related assets continue operating properly) and one or more corrective work orders (urgent formal requests for immediate repair, replacement, or remediation activities that address the underlying deficiencies and failed inspection points that originally triggered the need for interim life safety measures, including tasks such as permanent equipment repairs, system restorations, compliance corrections, or infrastructure improvements that will eventually eliminate the need for temporary ILSM protections), with both types of work orders containing detailed specifications, resource requirements, priority levels, and completion timelines. For example, when the ILSM determining subsystem 218 selects the interim measure “implement 24-hour fire watch patrol with portable smoke detectors” for ICU Smoke Compartment A due to failed smoke detector SMK-ICU-022, the work order generating subsystem automatically generates maintenance work order MWO-2024-0156 specifying “Weekly calibration and battery replacement for portable smoke detectors PSD-001 through PSD-008, estimated duration: 4 hours, assigned to: Fire Safety Technician, priority: routine maintenance, completion deadline: every Friday” to ensure the temporary detection equipment remains operational, and simultaneously generates corrective work order CWO-2024-0089 specifying “Replace and commission permanent smoke detector SMK-ICU-022 with new photoelectric sensor meeting 1.5% obscuration sensitivity specification, estimated duration: 6 hours, assigned to: Licensed Fire Systems Contractor, priority: high urgency, completion deadline: within 10 business days” to permanently resolve the underlying detection system failure, thereby ensuring that both the temporary interim protection measures are properly maintained and the permanent corrections are systematically planned and executed to restore full fire safety system functionality.

The work order generating subsystem is further configured to determine the status of the one or more maintenance work orders that is indicated as not completed and a status of the one or more corrective work orders that comprise one or more reported deficiencies requiring an immediate corrective action. The work order generating subsystem determines the status of work orders by performing systematic status tracking analysis that continuously monitors and evaluates the completion progress of two specific categories of work orders: the one or more maintenance work orders that is indicated as not completed (maintenance work orders that have been generated and assigned but remain in active, pending, or in-progress status rather than closed or finished status, meaning the scheduled maintenance activities supporting ILSM implementation have not yet been fully executed, such as “portable smoke detector battery replacement scheduled for Friday but not yet performed,” “fire watch equipment calibration assigned to technician but still pending,” or “emergency lighting inspection initiated but not completed”), and the one or more corrective work orders that comprise one or more reported deficiencies requiring an immediate corrective action (corrective work orders that contain urgent repair or remediation tasks addressing critical safety deficiencies that demand immediate attention due to their severity, regulatory compliance implications, or potential impact on occupant safety, such as “permanent smoke detector replacement required within 48 hours due to complete system failure,” “fire sprinkler pressure restoration needed immediately due to total suppression loss,” or “emergency egress door repair required urgently due to life safety code violation”), where status determination involves querying work order databases, analyzing completion timestamps, evaluating priority classifications, and identifying overdue or critical tasks. For example, the subsystem determines status by identifying maintenance work order MWO-2024-0156 “Weekly portable smoke detector calibration” shows status as “NOT COMPLETED—assigned to Fire Safety Technician on Monday, currently 3 days overdue, impacting ILSM effectiveness in ICU Compartment A,” and corrective work order CWO-2024-0089 “Replace permanent smoke detector SMK-ICU-022” shows status as “REPORTED DEFICIENCY REQUIRING IMMEDIATE CORRECTIVE ACTION—critical priority due to complete detection system failure, regulatory compliance violation, 24 vulnerable ICU patients at risk, must be completed within 48 hours to prevent facility citation,” thereby enabling the work order generating subsystem to identify which maintenance activities are falling behind schedule and potentially compromising interim protection effectiveness, and which corrective actions involve urgent deficiencies that require immediate escalation and resource allocation to protect facility occupants and maintain regulatory compliance.

In an aspect, the plurality of subsystems 114 further includes a profile retrieving subsystem (not shown in FIG. 2), that is communicatively connected to the one or more hardware processors. The profile retrieving subsystem is configured to retrieve the risk profile corresponding to the location environment associated with each of the one or more assets in each of the one or more smoke compartments, from the database 104. The risk profile comprises at least one of the potential risk factors, the plurality of risk levels, plurality of risk assessment scores, and the aggregated score. The profile retrieving subsystem performs systematic database query operations by executing targeted data retrieval processes that systematically locate, extract, and obtain a risk profile (a comprehensive data record containing consolidated risk assessment information and safety analysis results) corresponding to the location environment (the specific physical, operational, and contextual characteristics of the area where each asset is installed, including factors such as room type, occupancy classification, environmental conditions, proximity to high-risk areas, accessibility for maintenance, and operational criticality) associated with each of the one or more assets in each of the one or more smoke compartments, where this risk profile comprises at least one of four critical data components: the potential risk factors (identified hazards, vulnerabilities, and safety concerns that could contribute to fire incidents or compromise life safety, such as “high patient density,” “flammable materials present,” or “limited egress access”), the plurality of risk levels (categorized risk classifications such as “low risk,” “moderate risk,” “high risk,” or “critical risk” assigned to each smoke compartment based on comprehensive risk analysis), plurality of risk assessment scores (numerical values assigned to individual inspection points reflecting the importance and potential harm when specific assets fail inspections, such as “smoke detector inspection point score: 85,” “sprinkler system inspection point score: 92”), and the aggregated score (the cumulative numerical value representing total risk impact from all failed inspection points within each smoke compartment). For example, when retrieving the risk profile for Fire Sprinkler Asset SPR-ICU-015 located in ICU Smoke Compartment A, the profile retrieving subsystem queries the database and retrieves risk profile RP-ICU-A-SPR015 containing: potential risk factors including “high-vulnerability patient population (24 ICU patients on life support), limited mobility for evacuation, presence of oxygen-rich environment, 24/7 occupancy with critical medical equipment,” plurality of risk levels showing “smoke compartment classification: CRITICAL RISK due to patient vulnerability and life support dependency,” plurality of risk assessment scores including “sprinkler pressure inspection point: 92 points, sprinkler coverage inspection point: 88 points, sprinkler response time inspection point: 85 points,” and aggregated score showing “current compartment aggregated score: 385 points (exceeds threshold of 300 points),” thereby providing comprehensive risk context that enables the work order generating subsystem to understand the complete safety profile and prioritize maintenance and corrective work orders based on the specific risk characteristics and safety requirements of each asset's location environment.

Following the previous step, the work order generating subsystem is further configured to prioritize each of at least of: the one or more maintenance work orders and the one or more corrective work orders, based on the risk profile. The work order generating subsystem prioritizes each of at least the one or more maintenance work orders and the one or more corrective work orders by performing systematic ranking analysis that evaluates and orders work requests according to their relative importance, urgency, and safety impact using the comprehensive risk profile data (the consolidated risk assessment information containing potential risk factors, risk levels, risk assessment scores, and aggregated scores associated with each asset's location environment) as the primary decision-making criteria, where prioritization means assigning numerical priority rankings, urgency classifications, and resource allocation levels that determine the sequence and speed with which work orders should be executed, with higher-risk profiles receiving higher priority assignments to ensure that maintenance and corrective actions addressing the most critical safety concerns are completed first. For example, when prioritizing work orders for ICU Smoke Compartment A, the work order generating subsystem analyzes risk profiles and assigns Priority Level 1 (CRITICAL— complete within 24 hours) to corrective work order CWO-2024-0089 “Replace permanent smoke detector SMK-ICU-022” because its risk profile shows “CRITICAL RISK compartment classification, 24 vulnerable ICU patients, aggregated score 385 exceeding threshold 300, complete detection system failure,” assigns Priority Level 2 (HIGH—complete within 72 hours) to maintenance work order MWO-2024-0156 “Calibrate portable smoke detectors supporting ILSM implementation” because its risk profile indicates “supporting critical interim protection for high-vulnerability population,” and assigns Priority Level 3 (MODERATE—complete within 1 week) to maintenance work order MWO-2024-0157 “Inspect emergency lighting in administrative office” because its risk profile shows “LOW RISK compartment classification, standard occupancy, aggregated score 150 below threshold 200, non-critical support function,” thereby ensuring that work orders addressing assets in higher-risk environments with more vulnerable populations, higher aggregated risk scores, and more critical safety functions receive immediate attention and resources, while lower-risk maintenance activities are scheduled appropriately but do not delay critical life safety corrections.

The plurality of subsystems 114 further includes the output subsystem 222 that is communicatively connected to the one or more hardware processors 110. The output subsystem 222 is configured to provide the one or more controlled activities of the one or more assets within each of the one or more smoke compartments, to the one or more users through the one or more user interfaces associated with the one or more electronic devices 106 of the one or more users. The output subsystem 222 performs comprehensive information delivery operations by systematically presenting and transmitting one or more controlled activities (detailed records and real-time status information documenting the specific automated actions, operational changes, system adjustments, and protective measures that have been executed by the IoT controllers on fire safety assets, including activities such as “smoke detector sensitivity adjusted to 1.5% obscuration,” “emergency ventilation activated at 150% capacity,” “fire sprinkler system armed for immediate activation,” “emergency lighting switched to continuous operation,” or “fire doors configured for controlled closure sequence”) of the one or more assets within each of the one or more smoke compartments to one or more users (facility managers, fire safety officers, maintenance technicians, nursing supervisors, security personnel, and other authorized individuals responsible for facility safety oversight and emergency response coordination) through one or more user interfaces (interactive display screens, mobile applications, web-based dashboards, alert notification systems, and communication platforms that present information in accessible, organized, and actionable formats) associated with one or more electronic devices (computers, tablets, smartphones, wall-mounted displays, and specialized monitoring equipment) of the one or more users, where this output delivery ensures that relevant personnel receive timely, accurate, and comprehensive information about automated ILSM implementation and asset control status. For example, the output subsystem 222 provides controlled activities information by displaying on Fire Safety Manager John Smith's tablet interface “ICU Smoke Compartment A—Controlled Activities Status: (1) Smoke Detector SMK-ICU-022 sensitivity automatically adjusted from 2.0% to 1.5% obscuration at 14:23:15, (2) HVAC System HVC-ICU-025 emergency ventilation activated at 150% exhaust rate at 14:23:45, (3) Emergency Lighting EML-ICU-008 switched to continuous 100% brightness at 14:24:10, (4) Fire Sprinkler SPR-ICU-015 armed for automatic activation with 30-second delay at 14:24:30,” while simultaneously sending mobile app notifications to Nursing Supervisor Mary Johnson's smartphone showing “ILSM Active: ICU-A protected by enhanced detection+emergency ventilation, 24 patients monitored, all systems operational,” and displaying on the main fire command center wall monitor “Real-time Asset Control Dashboard: ICU Compartment A—4 assets under automated ILSM control, risk level reduced from CRITICAL to MODERATE, estimated protection duration 10-14 days,” thereby ensuring that all relevant personnel receive appropriate levels of detailed information about the controlled activities through their preferred electronic devices and user interfaces, enabling informed decision-making and coordinated emergency response.

In another aspect, the output subsystem 222 is configured to provide one or more notifications corresponding to the status of the created at least one of: the one or more maintenance work orders and the one or more corrective work orders, as the output, to the one or more electronic devices 106 associated with the one or more users. The one or more notifications inform the one or more users to provide instructions for generating at least one of: the one or more maintenance work orders or the one or more corrective work orders and inform the one or more users to monitor the status. In an exemplary embodiment, the output subsystem 222 is configured to generate the one or more notifications that the determined ILSM is no longer needed when at least one of the one or more work orders and the one or more corrective work orders associated with the generated ILSM are completed.

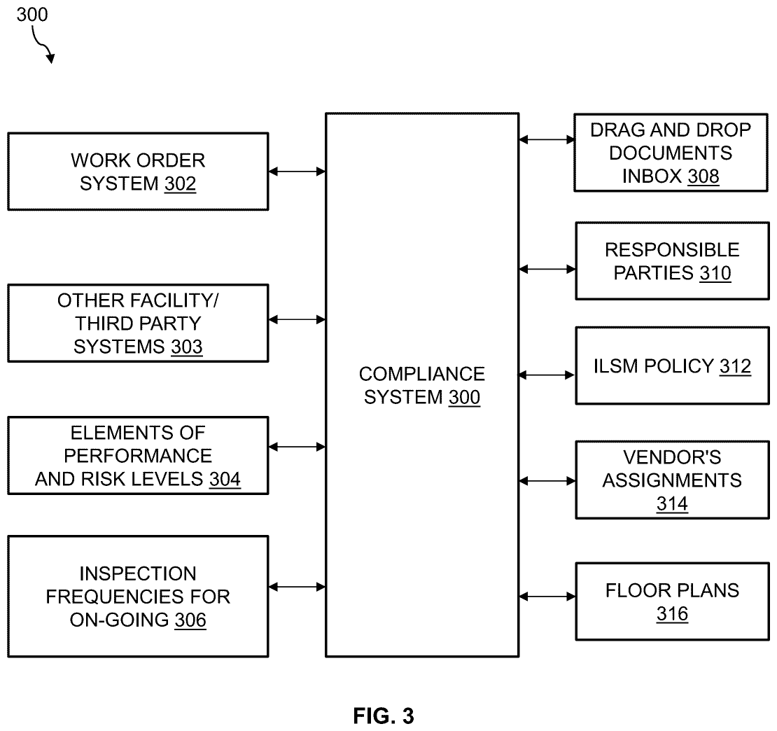

FIG. 3 illustrates an exemplary block diagram representation of an enterprise mobile, a cloud-based compliance system 300 (i.e., computer-implemented system 102) to increase hospital facility management compliance and to improve management and maintenance of healthcare facilities, in accordance with an embodiment of the present disclosure. Embodiments of the present disclosure integrate with multiple sources of existing data to determine where the facility is in terms of compliance, what needs to be performed, and provide the facility a schedule of what needs to be completed. To perform these functions, the compliance system 300 is initially set up to operate in a specific environment as discussed below. The smoke compartments in hospitals include, but are not limited to, patient care areas, operating rooms, operating rooms, emergency department, intensive care unit (ICU), imaging and diagnostic areas, imaging and diagnostic areas, and the like.

The patient care areas in hospitals typically have multiple smoke compartments to separate patient care areas, such as hospital rooms, nursing stations, and treatment rooms, from other areas of the building, such as administrative offices, lobbies, and mechanical rooms. Further, operating rooms are often located within a smoke compartment that is separated from other areas of the hospital to prevent the spread of smoke and fire. Furthermore, emergency department of a hospital may have a smoke compartment to separate the treatment area from other parts of the building, such as waiting rooms and administrative offices. Additionally, the intensive care unit (ICU) may be often located within a smoke compartment to protect critically ill patients from smoke and fire. The imaging and diagnostic areas, such as radiology and magnetic resonance imaging (MRI) rooms, may be located within a smoke compartment to prevent the spread of smoke and fire to other areas of the hospital. Smoke compartments in hospitals are designed to protect patients, staff, and visitors from the dangers of smoke and fire, and are typically required by building codes and fire safety regulations.

The set up to operate in a specific environment includes integrating the compliance and risk assessment system 300 with a work order system 302 associated with the facility, other facility/third party systems 303, providing elements of performance (EP) 304 and associated risk assessment scores based on mandated frequencies and risk levels, providing on-going inspection frequencies 306 associated with the facility such as a hospital, and setting up an inbox 308 for documents emailed to the compliance system 300. In addition, responsible parties 310 are associated with each EP, an interim life safety measures policy 312 is provided to the compliance system 300, as are vendor assignments 314 and floor plans 316 for the associated facility.

Mandated standards include a plurality of elements of performance (EPs) to indicate what needs to be completed to satisfy the standard. An example EP could be “the fire doors need to be inspected once per year. Embodiments of the present disclosure look not only at what frequency an asset should be inspected, but also how the inspection should be performed (what should be the inspection points). Each inspection point indicates a requirement of the respective EP. For example, an inspection point can be an asset inspection, a document, or an action to be taken. More specifically, an inspection point is any requirement relating to assets, machinery, instruments and other facility optional equipment, facility condition inspections, construction status and/or inspections relating to having valid current supporting document, as they relate to environmental care, life safety, and emergency management.

If any of those inspection points is such that risk assessment is required embodiments of the present disclosure determine whether a life safety measure should be in place. As inspections are performed, the associated documentation automatically files in the right place. In addition, if an inspection result requires a work order to be created, the compliance system 300 communicates with the work order system 302 to file a work order into the work order system 302 and then tracks the work order until it is completed. For actions performed by vendors outside the system, the vendors can email their report to the system and the report can be attached to a particular EP.

Embodiments of the present disclosure can be realized using an enterprise mobility system, wherein users utilize mobile assets, such as tablets and smart phones, to interact with the data and computer programs provided via the embodiments of the present disclosure. In general, the enterprise mobility nature of the embodiments of the present disclosure allows for increased productivity and decreased expenses for the facility.

In addition, embodiments of the present disclosure can be realized via Internet-based computing delivered to the facility's computers and devices through the Internet, often referred to as cloud-based computing. This allows embodiments to be accessed and shared as virtual resources in a secure and scalable manner, which further supports enterprise mobility as described above. In some aspect, embodiments of the present disclosure can be accessed and delivered via the Internet, instead of a local hard drive. In this manner, the related infrastructure of the compliance system 300 can be maintained by a provider, instead of the health facility itself.

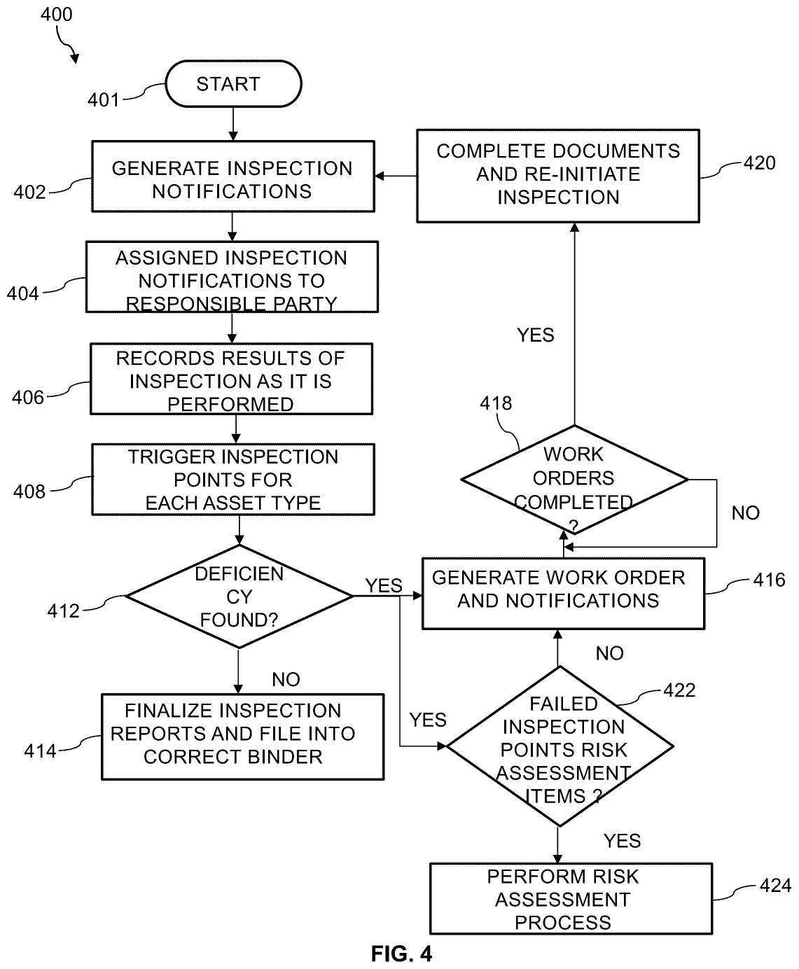

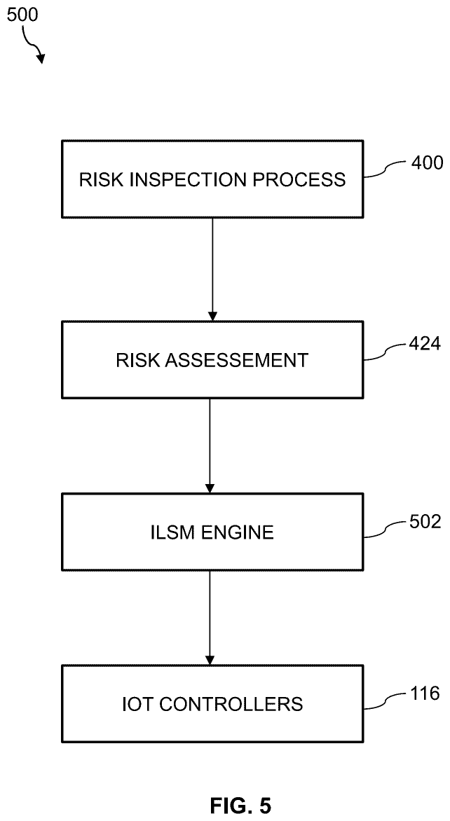

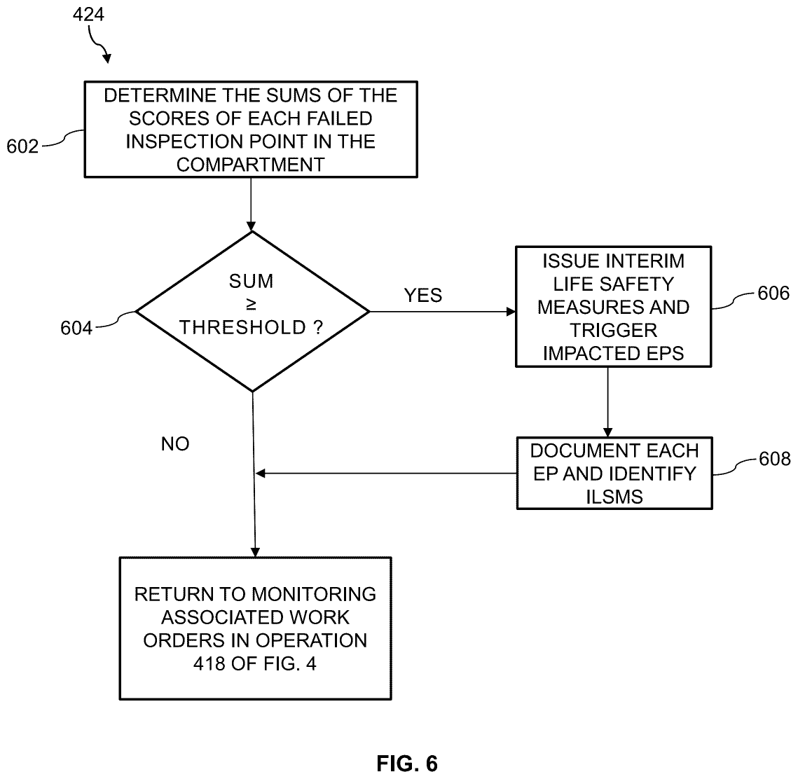

FIG. 4 illustrates an exemplary flow diagram representation of an inspection method 400 for compliance and risk assessment, in accordance with an embodiment of the present disclosure.

At step 401, initialization and set up operations are performed. Initialization and set up operations can include, for example, integrating the compliance system with a facility's work order system, providing EP and associated risk assessment scores, providing the hospital's on-going inspection frequencies, associating responsible parties with each EP, providing an interim life safety measures policy, determining vendor assignments, and uploading facility floor plans.

At step 402, inspection notifications are generated, and, at step 404, these notifications are assigned to in-house responsible party or an outside vendor. Based on a predetermined frequency, inspection notifications for EPs are generated by the compliance system. In one embodiment, the compliance system shows the user the standard requirements and the related elements of performance. Beside each EP the user is shown the mandated frequency and the specific person assigned to each EP.

At step 406, the system records the results of the inspection as it is performed. During the inspection, inspection points are triggered for each asset type, at step 408. In addition, rounds and drills are recorded with respect to whether they have been performed. Embodiments of the present disclosure analyze each requirement of the JC to determine if the requirement relates to an asset inspection, a document, or if the requirement relates to a miscellaneous action that must be performed, such as filing rounds that performed every day.

A determination is then made as to whether a deficiency has been found in any inspection point, at step 412. If no deficiency is found in any inspection point, the method 400 continues to step 414. Otherwise, the method 400 branches to step 416 and step 422.

At step 414, the inspection reports are finalized and filed into the correct binder. During this operation, the inspection reports are e-signed, and the inspection points are reset to the next inspection date. In one embodiment, work from vendors, policies that must be reviewed, etc. are all stored in a repository that is used to show the user whether they have satisfied each requirement. If any requirement is not satisfied, the user can view into the repository and view what is upcoming, what is due, what is past due, where they are now, and how does that affect them in terms of the inspection that will be performed by the JC.

In one embodiment, the compliance system 300 has a plurality of binders. When documents are attached to the compliance system 300, the document is automatically filed into the appropriate binder. Items emailed to the system are sent to the inbox, as indicated in FIG. 3. From this inbox, email attachments can be attached to a particular EP. Each EP has its own document requirements indicating what the document needs to include to satisfy that EP. These requirements can include such aspects as, for example, a signature and/or a date. When a user attempts to attach a document to an EP, embodiments present a number of questions to the user based on what is required to satisfy the EP's document requirements. For the document to be accepted, the user must answer in the affirmative for each question.

If deficiencies are found in any inspection point, the method 400 branches to step 416 and step 422. In one embodiment, whenever an inspection point that relates to a risk assessment item fails inspection, the user is given a choice of whether to have “Time to Resolve” or to “Mark as deficiency.” When the user selects “Time to Resolve,” the user is given a predetermined amount of time, for example four hours, to resolve the inspection point deficiency before the inspection point failure result is transferred to steps 416 and 422. If the user resolves the deficiency within the predetermined time, the inspection point failure is not transferred, and the method moves to step 414. Otherwise, the inspection point failure is transferred to steps 416 and 422. If the user selects “Mark as deficiency,” the inspection point failure is immediately transferred to steps 416 and 422.

At step 416, a work order and notifications are created. As mentioned previously, embodiments of the present disclosure are initially integrated with a work order system. At step 416, the compliance system communicates with the work order system to create one or more work orders to correct the deficiencies found during the inspection. The compliance system then monitors these work orders to determine when they are completed.

At step 418, a determination is made as to whether the work orders are completed. If the work orders are not completed, the method 400 loops to another step 418 as it continues to monitor the work orders. If the work orders are completed the method 400 continues to step 420, where the documents are completed, and the inspection is reinitiated.

In addition to performing step 416, the method 400 branches to step 422 when deficiencies are found in any inspection point. At step 422, a decision is made as to whether any of the failed inspection points are risk assessment items. If none of the failed inspection points are risk assessment items, no additional actions are performed, and the method continues to generate work orders and monitor them as discussed above with respect to steps 416 and 418. Otherwise, a risk assessment may be performed at step 424.

FIG. 5 illustrates an exemplary block diagram representation of elements of a compliance and risk assessment computer program 500, in accordance with an embodiment of the present disclosure. The computer program 500 includes computer instructions that perform the inspection method 400 described previously with respect to FIG. 4, computer instructions that perform risk assessment 424 as described with reference to FIG. 6, and an ILSM engine 502 that issues Interim Life Safety Measures (ILSMs).

As noted above, during the inspection method 400, whenever an inspection point that relates to a risk assessment item fails inspection, the user is given a choice of whether to have “Time to Resolve” or to “Mark as deficiency.” When the user selects “Time to Resolve,” the user is given a predetermined amount of time, for example four hours, to resolve the inspection point deficiency before the inspection point failure result is transferred to risk assessment 424. If the user resolves the deficiency within the predetermined time, the inspection point failure is not transferred to risk assessment 424, otherwise the inspection point failure is transferred to risk assessment 424. If the user selects “Mark as deficiency,” the inspection point failure is immediately transferred to risk assessment process 424. In risk assessment 424, a determination is made as to whether failed points of inspection of the EP data indicate that the inspection point failure should be sent to the ILSM engine 502, where an ILSM is issued if required. Further, when the corresponding ILSM is determined for each of the one or more assets within each of the one or more smoke compartments, the plurality of IoT controllers 116 configured in the one or more assets is activated by automatically performing corresponding one or more actions to control the one or more assets within each of the one or more smoke compartments to protect the one or more individuals in the one or more smoke compartments of the facility.

Embodiments of the present disclosure analyze each specific requirement of the standard ILSM to create the process required within each function. In addition, the facility's own ILSM processes, as determined in their own policy, can be added to the ILSM engine. In addition to creation via assets, ILSMs can also be triggered by events, such as construction. These additional events are determined during set up of the compliance and risk assessment system based on existing standards and the requirements of the particular facility using the present disclosure.

FIG. 6 illustrates an exemplary flow diagram representation of a process for risk assessment 424 in the compliance system 300, in accordance with an embodiment of the present disclosure. During an inspection, if a deficiency is found in an inspection point that is a risk assessment item, the risk assessment process 424 is performed. In the compliance system 300, particular inspection points require risk analysis, and thus are given a score based on the importance and the harm created when the inspection point fails inspection.

At step 602, all deficiencies in a particular compartment are collected and the sums of the scores of each failed inspection point in the compartment is calculated. Each facility is subdivided into a plurality of compartments. These compartments can be of any size and are usually contained within a single floor of a facility. A compartment can comprise a single room, a portion of a room, a plurality of rooms, or any other combination that facilitates risk assessment for the facility. For example, as mentioned previously, floor plans of the facility are uploaded to the system during initialization. Each floor plan indicates the location of assets on the floor and shows the location of each compartment that makes up the floor. As assets are inspected, any deficiencies within the compartment are tracked. Deficiencies for items that require risk assessment are collected for each compartment, and the sums of failed risk assessment items scores are calculated for each compartment.

It should be noted that embodiments of the present disclosure can allow the user to drag and drop assets information into an icon created which can be then dragged and dropped on to the floor plan, select an asset on the floor plan to show all the details for the asset, which comes from the integrated work order system or can be manually added. The user can then inspect the asset, since the compliance system shows all the inspection points that are relevant to that type of asset. For example, a fire door can have certain inspection points and the compliance system shows whether they comply. Some of the inspection points include a score. These inspection points are required to go through a risk assessment to determine if interim safety measures are required.

A decision is then made, at step 604, as to whether the sum of the scores of each failed inspection point in a compartment equals or exceeds a predetermined threshold for the compartment. If the sum does not equal or exceed the predetermined threshold for the compartment, no additional action is required, and the method continues to generate work orders and monitor them as discussed above with respect to steps 416 and 418. However, if the sum equals or exceeds the predetermined threshold for the compartment the method 424 branches to step 606 where one or more interim life safety measure are issued. At step 608, the method 424 includes documenting each EP and identifying ILSMs.

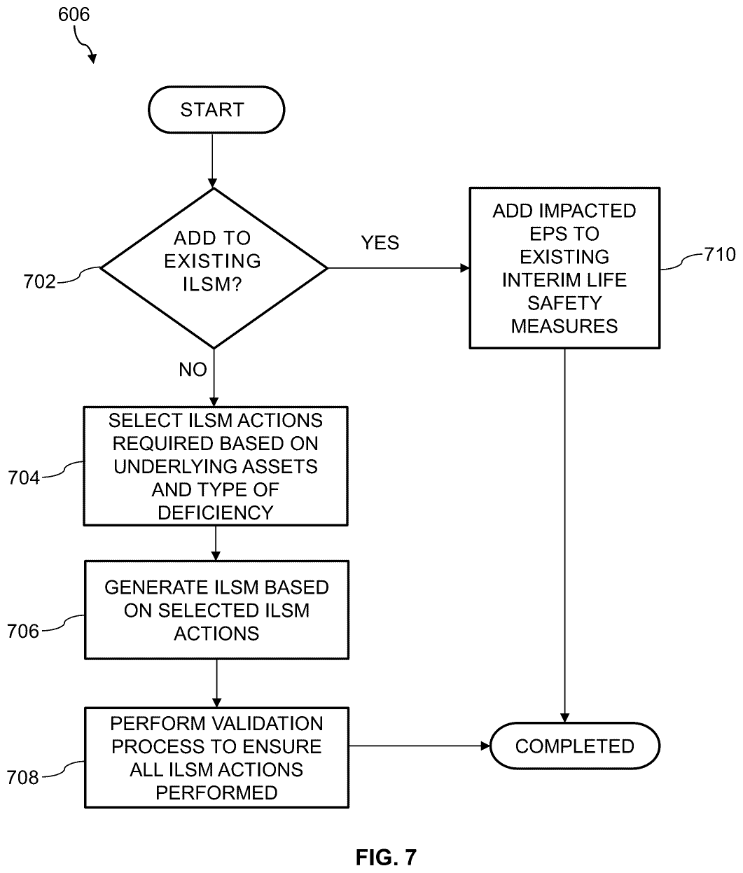

FIG. 7 illustrates an exemplary flow diagram representation of a method 606 for issuing an ILSM, in accordance with an embodiment of the present disclosure. Because an existing ILSM may already be in place, a determination is made, at step 702, as to whether to create a new ILSM or add the asset that created the ILSM to an existing ILSM. If a new ILSM is to be generated, the method 606 continues to step 704, otherwise the method 606 branches to step 710 where the impacted EPs are added to an existing ILSM.

At step 704, ILSM actions are selected based on the underlying assets and the type of deficiencies involved. Embodiments of the present disclosure store in a database a list of ILSM actions defined by accrediting bodies and the particular facility that, when put into action, protect the safety and health of patients by compensating for hazards caused by Life Safety Code deficiencies or construction activity. In addition to the list of ILSM actions, embodiments store in a database correlating relationship between the types of deficiencies that may be encountered and the ILSM actions that address these deficiencies. Embodiments of the present disclosure then automatically identify and determine, based on the asset deficiencies, ILSM actions to be taken.

Embodiments of the present disclosure then generate an ILSM based on the selected ILSM actions, in operation 706. The ILSM is a list of selected ILSM actions that are to be performed. As noted above, ILSMs are health and safety measures put in place to protect the safety of patients, visitors, and staff who work in the hospital. ILSMs can, for example, take the form of exit signs and pathways to an egress point, fire protection systems including smoke detectors, fire suppression, fire extinguishers and fire alarm systems, smoke barriers, emergency evacuation plans, in addition to many other items that contribute to the well-being and safety of occupants in the hospital or healthcare facility. Construction or maintenance activities can have an impact on the life safety systems in the hospital, thus requiring an Interim plan to address the deficiencies created by the work activity.

When an ILSM is issued, ILSM actions, which are required steps, are selected from a pre-defined list, and documented. For example, ILSM actions that may be suggested by ILSM engine might include for example:
(a) Provides temporary but equivalent fire alarm and detection systems for use when a fire system is impaired.
(b) Post signage identifying the location of alternative exits to everyone affected a picture of the posted signage can be taken and each of the item provide for the ability to take pictures and enter notes.
(c) Enforces storage, housekeeping, and debris-removal practices that reduce the building's flammable load.
(d) Enforces storage, housekeeping, and debris-removal practices that reduce the building's flammable load.
(e) debris removal
(f) Uses temporary construction partitions that are smoke-tight or made of noncombustible or limited-combustible material that will not contribute to the development or spread of fire.
(g) when the hospital identifies Life Safety Code deficiencies that cannot be immediately the hospital either evacuates the building or
(h) Initiates a fire watch and
(i) Other pre-defined items.

At step 708, a validation process is performed to ensure all ILSM actions are performed. The ILSM engine of the embodiments of the present disclosure provides the user with the ability to document each ILSM action taken and indicate as performed. Once an ILSM is created, the validation process is activated to confirm that all documents and all required action were performed. Alerts are issued if ILSM actions are not performed within the required timeframe. Alerts also are issued at each step of the process, upon deficiency discovery, upon generation of ILSM, and upon actions taken based on ILSM requirements, which provides full documentations of action item and steps taken and are reaffirmed, based on current ILSM incident duration. The ILSM engine 502 also monitors the work order system for completion of all work orders underlying as ILSM incident. Once the work orders are completed, it allows for closing of that ILSM incident, as discussed subsequently.

Referring back to FIG. 6, during an inspection, if an item requires risk assessment the item will have a score. Embodiments of the present disclosure examine all the items which are risk assessment items, put them in a holding area, and then determine whether all those combined within a particular compartment reach a threshold.

For example, on an exemplary floor within a hospital, there exist a plurality of small compartments. Within one small compartment there may be, for example, one item that has a score of 3 and another item that has a score of 4. In this example, the compartment has a threshold of 6. In this example, since the sum of the failed risk assessment items is 7, which exceeds the compartment threshold of 6, an ILSM is issued. If only one of these are present, then an ILSM is not issued because the sum did not exceed the compartment threshold of 6.

Embodiments of the present disclosure utilized the fourteen elements of performance mandated by the JC that are required to be addressed. The compliance system of the embodiments of the present disclosure issues these items, thus requiring the user to address the items. At step 608, each of these items is required to be documented that “yes” the user has taken an action on the item. Thereafter, the process 424 continues to monitor the associated work orders in operation 418 of FIG. 4. Once those work orders are closed, the deficiencies are closed, and the ILSM is no longer required. Thereafter, the assets are posted again for re-inspection in operation 420 of FIG. 2.

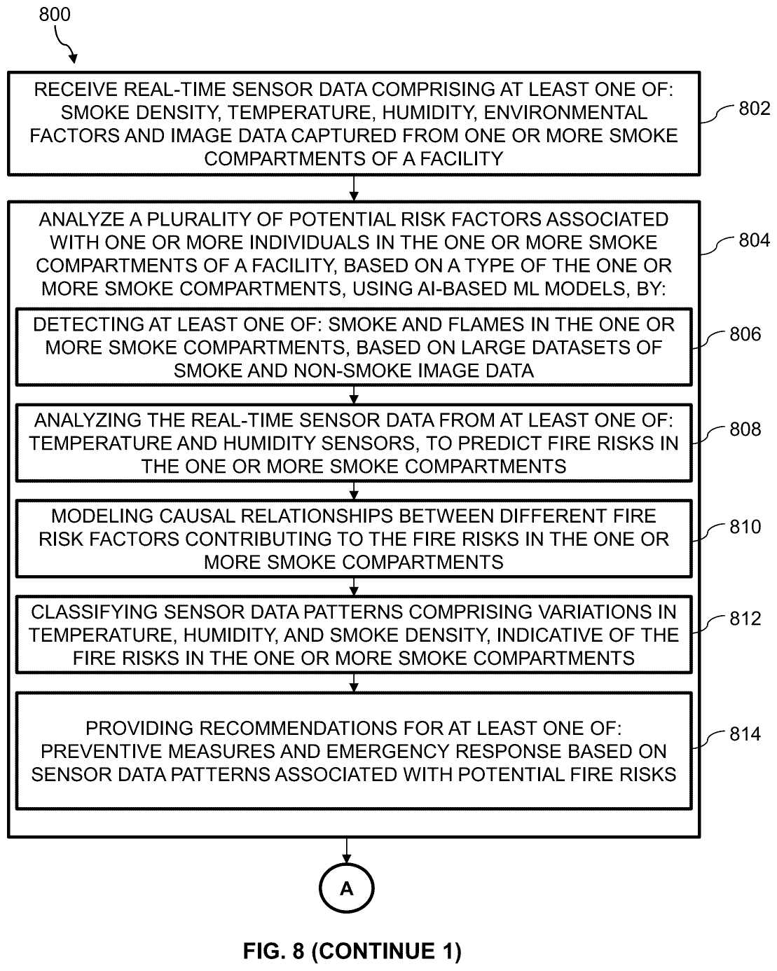

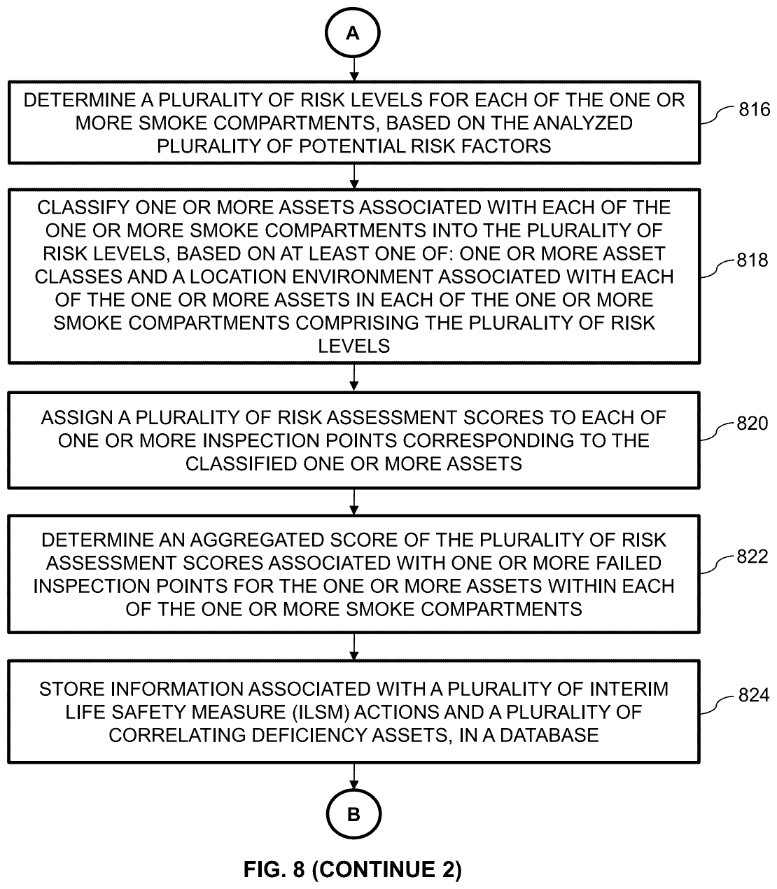

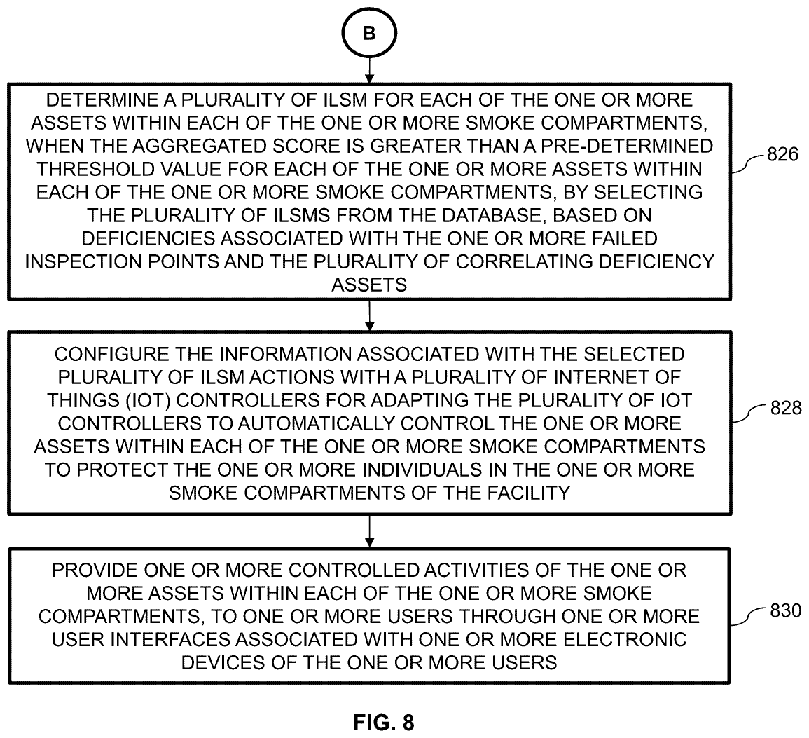

FIG. 8 illustrates a flow chart depicting a computer-implemented method 800 for determining the interim life safety measure (ILSM) to automatically control the one or more assets within the one or more smoke compartments in the facility, according to an example embodiment of the present disclosure.

At step 802, the computer-implemented method 800 may include receiving, by the one or more hardware processors 110 associated with the computer-implemented system 102, the real-time sensor data comprising at least one of: smoke density, temperature, humidity, environmental factors and image data captured from the one or more smoke compartments of the facility.

At step 804, the computer-implemented may further include analyzing, by the one or more hardware processors 110, a plurality of potential risk factors associated with one or more individuals in the one or more smoke compartments of the facility, based on a type of the one or more smoke compartments. Analyzing the plurality of potential risk factors, comprises utilizing, by the one or more hardware processors, the one or more trained AI-based machine learning models, by at least one of: (a) detecting, by the one or more hardware processors, at least one of: the smoke and flames in the one or more smoke compartments, based on the large datasets of the smoke and non-smoke image data, as shown in step 806, (b) analyzing, by the one or more hardware processors, the real-time sensor data from at least one of: the temperature and humidity sensors, to predict the fire risks in the one or more smoke compartments, as shown in step 808, (c) modeling, by the one or more hardware processors, the causal relationships between the different fire risk factors contributing to the fire risk in the one or more smoke compartments, as shown in step 810, (d) classifying, by the one or more hardware processors, the sensor data patterns comprising the variations in temperature, humidity, and smoke density, indicative of the fire risk in the one or more smoke compartments, as shown in step 812, and (e) providing, by the one or more hardware processors, the recommendations for at least one of: preventive measures and emergency response based on the sensor data patterns associated with potential fire risks, as shown in step 814. In an embodiment, the one or more trained AI-based machine learning models comprise at least one of: first convolutional neural networks (CNNs), recurrent neural networks (RNNs), Bayesian networks, first support vector machines (SVMs), decision trees, and the like.

At step 816, the computer-implemented method 800 may further include determining, by the one or more hardware processors 110, the plurality of risk levels for each of the one or more smoke compartments, based on the analyzed plurality of potential risk factors.

At step 818, the computer-implemented method 800 may further include, in response to determining the plurality of risk levels, classifying, by the one or more hardware processors 110, the one or more assets associated with each of the one or more smoke compartments into the plurality of risk levels, based on at least one of: the one or more asset classes and the location environment associated with each of the one or more assets in each of the one or more smoke compartments comprising the plurality of risk levels. The one or more asset classes are generated using at least one of: asset class inheritance-based AI models and asset class acquisition-based AI models. At least one of: the asset class inheritance-based AI models and asset class acquisition-based AI models is trained to: (a) analyze image data and historical data associated with the one or more assets, and (b) classify the one or more assets into asset classes based on the analyzed image data and historical data. At least one of the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, Generative Adversarial Networks (GANs), and the like.

At step 820, the computer-implemented method 800 may further include, assigning, by the one or more hardware processors 110, a plurality of risk assessment scores to each of the one or more inspection points corresponding to the classified one or more assets. The risk assessment score corresponds to at least one of: the importance and the potential harm created when the one or more inspection point fails the inspection. Each of the one or more inspection points corresponds to the requirement of the element of performance (EP).

At step 822, the computer-implemented method 800 may further include determining, by the one or more hardware processors 110, the aggregated score of the plurality of risk assessment scores associated with the plurality of failed inspection points for the one or more assets within each of the one or more smoke compartments.

At step 824, the computer-implemented method 800 may further include storing, by the one or more hardware processors 110, the information associated with the plurality of Interim Life Safety Measure (ILSM) actions and the plurality of correlating deficiency assets, in the database 104. Each ILSM action defines the action related to protecting the one or more individuals at the facility. each correlating deficiency asset defines the correlation between the deficiency encountered at the facility and at least one ILSM action.

At step 826, the computer-implemented method 800 may further include determining, by the one or more hardware processors, one or more ILSMs (i.e., the plurality of ILSMs), when the aggregated score is greater than the pre-determined threshold value for each of the one or more assets within each of the one or more smoke compartments. The ILSM is the health and safety measure to protect the one or more individuals at the facility. In an embodiment, determining the plurality of ILSMs comprises selecting, by the one or more hardware processors 110, the plurality of ILSMs from the database, based on deficiencies associated with the one or more failed inspection points and the plurality of correlating deficiency assets.

At step 828, the computer-implemented method 800 may further include configuring, by the one or more hardware processors 110, the information associated with the selected plurality of ILSM actions with the plurality of internet of things (IoT) controllers 116 for adapting the plurality of IoT controllers 116 to automatically control the one or more assets within each of the one or more smoke compartments to protect the one or more individuals in the one or more smoke compartments of the facility. In an embodiment, each IoT controller of the plurality of IoT controllers 116 is configured in corresponding one or more assets within each of the one or more smoke compartments.

At step 830, the computer-implemented method 800 may further include providing, by the one or more hardware processors 110, the one or more controlled activities of the one or more assets within each of the one or more smoke compartments, as the output to the one or more users through the one or more user interfaces associated with the one or more electronic devices 106 of the one or more users.

The order in which the computer-implemented method 800 is described is not intended to be construed as a limitation, and any number of the described method blocks may be combined or otherwise performed in any order to implement the computer-implemented method 800 or an alternate method. Additionally, individual blocks may be deleted from the computer-implemented method 800 without departing from the spirit and scope of the present disclosure described herein. Furthermore, the computer-implemented method 800 may be implemented in any suitable hardware, software, firmware, or a combination thereof, that exists in the related art or that is later developed. The computer-implemented method 800 describes, without limitation, the implementation of the computer-implemented system 102 and 300. A person of skill in the art will understand that computer-implemented method 800 may be modified appropriately for implementation in various manners without departing from the scope and spirit of the disclosure.

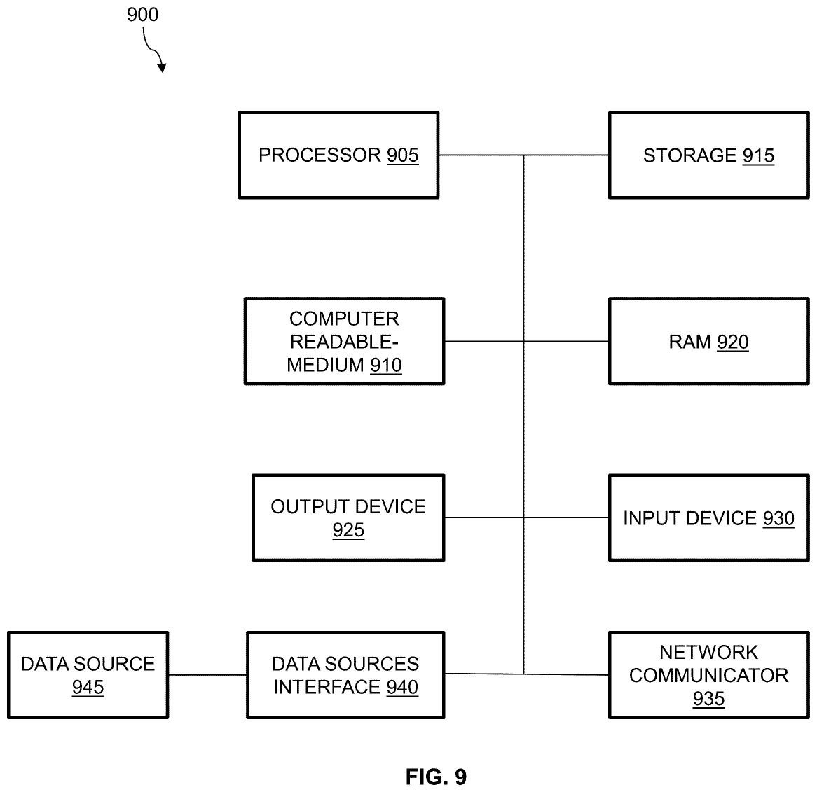

FIG. 9 illustrates an exemplary block diagram representation of a hardware platform 900 for implementation of the disclosed computer-implemented system 102, according to an example embodiment of the present disclosure. For the sake of brevity, the construction, and operational features of the computer-implemented system 102 which are explained in detail above are not explained in detail herein. Particularly, computing machines such as but not limited to internal/external server clusters, quantum computers, desktops, laptops, smartphones, tablets, and wearables which may be used to execute the computer-implemented system 102 or may include the structure of the hardware platform 900. As illustrated, the hardware platform 900 may include additional components not shown, and some of the components described may be removed and/or modified. For example, a computer system with multiple GPUs may be located on external-cloud platforms including Amazon Web Services, or internal corporate cloud computing clusters, or organizational computing resources.

The hardware platform 900 may be a computer system such as the computer-implemented system 102 that may be used with the embodiments described herein. The computer system may represent a computational platform that includes components that may be in a server or another computer system. The computer system may execute, by the processor 905 (e.g., a single or multiple processors) or other hardware processing circuit, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 905 that executes software instructions or code stored on a non-transitory computer-readable storage medium 910 to perform methods of the present disclosure. The software code includes, for example, instructions to generate time-based contextual graph in cloud computing environment. In an example, the potential risk analyzing subsystem 206, the risk level determining subsystem 208, the asset classifying subsystem 210, the score assigning subsystem 212, the aggregated score determining subsystem 214, the action storing subsystem 216, the Interim Life Safety Measure (ILSM) determining subsystem 218, the asset controlling subsystem 220, the output subsystem 222, the work order generating subsystem, and the profile retrieving subsystem, may be software codes or components performing these rules.

The instructions on the computer-readable storage medium 910 are read and stored the instructions in storage 915 or in random access memory (RAM). The storage 915 may provide a space for keeping static data where at least some instructions could be stored for later execution. The stored instructions may be further compiled to generate other representations of the instructions and dynamically stored in the RAM such as RAM 920. The processor 905 may read instructions from the RAM 920 and perform actions as instructed.

The computer system may further include the output device 925 to provide at least some of the results of the execution as output including, but not limited to, visual information to users, such as external agents. The output device 925 may include a display on computing devices and virtual reality glasses. For example, the display may be a mobile phone screen or a laptop screen. GUIs and/or text may be presented as an output on the display screen. The computer system may further include an input device 930 to provide a user or another device with mechanisms for entering data and/or otherwise interact with the computer system. The input device 930 may include, for example, a keyboard, a keypad, a mouse, or a touchscreen. Each of these output devices 925 and input device 930 may be joined by one or more additional peripherals. For example, the output device 925 may be used to display the results such as bot responses by the executable chatbot.

A network communicator 935 may be provided to connect the computer system to a network and in turn to other devices connected to the network including other clients, servers, data stores, and interfaces, for example. A network communicator 935 may include, for example, a network adapter such as a LAN adapter or a wireless adapter. The computer system may include a data sources interface 940 to access the data source 945. The data source 945 may be an information resource. As an example, a database of exceptions and rules may be provided as the data source 945. Moreover, knowledge repositories and curated data may be other examples of the data source 945.

Embodiments of the present disclosure provide the computer-implemented system 102 and method 800 for determining the interim life safety measure (ILSM) to automatically control the one or more assets within the one or more smoke compartments in a facility. The present disclosure provides the computer-implemented system 102 and method 800 that affords hospitals the ability to assure ongoing compliance and reduce cost by migrating from their current existing costly manual inspections to using a central repository and tracking solution that helps improve healthcare physical environment quality of service and provides for successful compliance outcomes. Embodiments of the present disclosure ensure Interim Life Safety Measures (ILSMs) are completed, while also ensuring and validating the required activities are performed on time. Thus, embodiments of the present disclosure schedule regulatory compliance activities around regulatory mandated timelines. Embodiments of the present disclosure also provide warnings and alarms when deadlines approach, give emergency notification to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall.

The computer-implemented system 102 employs sophisticated artificial intelligence architectures including convolutional neural networks (CNNs), recurrent neural networks (RNNs), Bayesian networks, support vector machines (SVMs), and decision trees to perform real-time analysis of multi-dimensional sensor data comprising smoke density, temperature, humidity, environmental factors, and image data, enabling automated smoke and flame detection based on large training datasets, predictive fire risk analysis through temporal pattern recognition, causal relationship modeling between interconnected fire risk factors, intelligent classification of sensor data patterns indicative of emerging threats, and proactive recommendations for preventive measures and emergency response protocols, while simultaneously utilizing asset class inheritance-based and acquisition-based AI models incorporating CNNs, random forests, LSTM networks, SVMs, and Generative Adversarial Networks (GANs) to automatically analyze historical performance data and classify assets into appropriate risk categories, thereby providing intelligent threat prediction, automated risk quantification, and adaptive learning capabilities that continuously improve accuracy and responsiveness compared to traditional reactive fire safety systems that rely on manual assessment and predetermined thresholds.

The computer-implemented system 102 further performs seamless translation of human-readable interim life safety measure (ILSM) actions into machine-executable control commands compatible with distributed IoT controllers using multiple communication protocols including wireless networks, wired connections, and mesh networking topologies, while implementing sophisticated fail-safe mechanisms that ensure automatic reversion to safe operational states during communication failures or system malfunctions, coordinating multi-controller operations through precisely synchronized timing sequences that manage interdependent fire safety assets across smoke compartments, dynamically updating controller configurations in real-time based on changing ILSM requirements and emergency conditions, and maintaining continuous operational monitoring through automated feedback collection, error reporting, and performance validation, thereby eliminating human intervention delays, reducing operator error risks, ensuring coordinated system responses, and providing adaptive automated control that maintains protective coverage even during system disruptions or evolving emergency scenarios without requiring manual reconfiguration or expert technical intervention.

The computer-implemented system 102 further integrates sophisticated aggregation algorithms including weighted summation, root mean square computations, and maximum value selection with temporal decay functions and compartment-specific weighting factors to generate precise aggregated risk scores that account for cumulative failure impacts, proximity-based risk amplification, and time-sensitive degradation patterns, while maintaining a comprehensive database of correlating deficiency assets that enables automated selection of optimal ILSM actions based on specific deficiency types, regulatory requirements, and facility characteristics, automatically generating detailed implementation plans with resource requirements, timelines, and effectiveness metrics, creating comprehensive audit logs documenting all control activities for regulatory compliance, generating prioritized maintenance and corrective work orders based on risk profiles, and providing multi-platform output delivery through mobile applications, web dashboards, and monitoring interfaces that ensure appropriate personnel receive timely, accurate information about system status and controlled activities, thereby enabling evidence-based decision making, ensuring regulatory compliance, facilitating proactive maintenance scheduling, and providing complete operational transparency that supports coordinated emergency response and systematic safety management without requiring manual documentation, subjective risk assessment, or reactive maintenance approaches.

One of ordinary skill in the art will appreciate that techniques consistent with the present disclosure are applicable in other contexts as well without departing from the scope of the disclosure.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, and the like. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of embodiments of the present disclosure. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the disclosure need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, and the like. of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based here on. Accordingly, the embodiments of the present disclosure are intended to be illustrative, but not limiting, of the scope of the disclosure, which is set forth in the following claims.

What is claimed is:

1. A computer-implemented system for determining an interim life safety measure (ILSM) to automatically control one or more assets within one or more smoke compartments in a facility, the computer-implemented system comprising:
one or more hardware processors; and
a memory coupled to the one or more hardware processors, wherein the memory comprises a set of program instructions in form of a plurality of subsystems that are configured to be executed by the one or more hardware processors, wherein the plurality of subsystems comprises:
a potential risk analyzing subsystem configured to:
receive real-time sensor data comprising at least one of: smoke density, temperature, humidity, environmental factors and image data captured from the one or more smoke compartments of the facility; and
analyze a plurality of potential risk factors associated with one or more individuals in the one or more smoke compartments of the facility, based on a type of the one or more smoke compartments, wherein in analyzing the plurality of potential risk factors, the potential risk analyzing subsystem is configured to utilize one or more trained Artificial Intelligence (AI)-based machine learning models, by at least one of:
detecting at least one of: smoke and flames in the one or more smoke compartments, based on large datasets of smoke and non-smoke image data;
analyzing the real-time sensor data from at least one of: temperature and humidity sensors, to predict fire risks in the one or more smoke compartments;
modeling causal relationships between one or more fire risk factors contributing to the fire risks in the one or more smoke compartments;
classifying sensor data patterns comprising variations in temperature, humidity, and smoke density, indicative of the fire risks in the one or more smoke compartments; and
providing recommendations for at least one of: preventive measures and emergency response based on the sensor data patterns associated with potential fire risks,
wherein the one or more trained AI-based machine learning models comprise at least one of: first convolutional neural networks (CNNs), recurrent neural networks (RNNs), Bayesian networks, first support vector machines (SVMs), and decision trees;
a risk level determining subsystem configured to determine a plurality of risk levels for each of the one or more smoke compartments, based on the analyzed plurality of potential risk factors;
in response to determining the plurality of risk levels, an asset classifying subsystem configured to classify one or more assets associated with each of the one or more smoke compartments into the plurality of risk levels, based on at least one of: one or more asset classes and a location environment associated with each of the one or more assets in each of the one or more smoke compartments comprising the plurality of risk levels,
wherein the one or more asset classes are generated using at least one of: asset class inheritance-based AI models and asset class acquisition-based AI models, wherein at least one of: the asset class inheritance-based AI models and asset class acquisition-based AI models is trained to:
analyze image data and historical data associated with the one or more assets; and
classify the one or more assets into asset classes based on the analyzed image data and historical data, and
wherein at least one of: the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, and Generative Adversarial Networks (GANs);
a score assigning subsystem configured to assign a plurality of risk assessment scores to each of one or more inspection points corresponding to the classified one or more assets, wherein the risk assessment score corresponds to at least one of an importance and a potential harm created when the one or more inspection points fail an inspection, and wherein each of the one or more inspection points corresponds to a requirement of an element of performance (EP);
an aggregated score determining subsystem configured to determine an aggregated score of the plurality of risk assessment scores associated with one or more failed inspection points for the one or more assets within each of the one or more smoke compartments;
an action storing subsystem is configured to store information associated with a plurality of Interim Life Safety Measure (ILSM) actions and a plurality of correlating deficiency assets, in a database, wherein each ILSM action defines an action related to protecting the one or more individuals at the facility, and wherein each correlating deficiency asset defines a correlation between a deficiency encountered at the facility and at least one ILSM action;
an Interim Life Safety Measure (ILSM) determining subsystem configured to determine a plurality of ILSMs for each of the one or more assets within each of the one or more smoke compartments, when the aggregated score is greater than a pre-determined threshold value for each of the one or more assets within each of the one or more smoke compartments, wherein the ILSM is a health and safety measure to protect the one or more individuals at the facility,
wherein in determining the plurality of ILSMs, the ILSM determining subsystem is configured to select the plurality of ILSMs from the database, based on deficiencies associated with the one or more failed inspection points and the plurality of correlating deficiency assets;
an asset controlling subsystem configured to configure the information associated with the selected plurality of ILSM actions with a plurality of internet of things (IoT) controllers for adapting the plurality of IoT controllers to automatically control the one or more assets within each of the one or more smoke compartments to protect the one or more individuals in the one or more smoke compartments of the facility, wherein each IoT controller of the plurality of IoT controllers is configured in corresponding one or more assets within each of the one or more smoke compartments; and
an output subsystem configured to provide one or more controlled activities of the one or more assets within each of the one or more smoke compartments, to one or more users through one or more user interfaces associated with one or more electronic devices of the one or more users.

2. The computer-implemented system of claim 1, wherein in determining the plurality of risk levels for each of the one or more smoke compartments, based on the analyzed plurality of potential risk factors, the risk level determining subsystem is configured to:
analyze one or more correlation patterns between the plurality of potential risk factors and historical fire incident data to determine risk weighting coefficients for each potential risk factor within each of the one or more smoke compartments;
apply the one or more trained AI-based machine learning models comprising at least one of: neural networks, decision trees, and regression models to process the analyzed plurality of potential risk factors and generate quantitative risk assessment values for each of the one or more smoke compartments;
categorize the quantitative risk assessment values into the plurality of risk levels comprising at least one of: low risk, moderate risk, high risk, and critical risk levels based on predetermined threshold ranges;
incorporate environmental context factors comprising at least one of: occupancy type, building age, construction materials, and facility usage patterns to adjust the plurality of risk levels for each of the one or more smoke compartments;
perform dynamic risk level re-computation in real-time based on changes in the real-time sensor data and updated potential risk factors to maintain current risk level assessments;
validate risk level accuracy by comparing determined risk levels against historical incident patterns and regulatory compliance standards for analogous smoke compartments; and
generate risk level confidence scores indicating the reliability of each determined risk level based on data quality, sensor accuracy, and completeness of the analyzed plurality of potential risk factors.

3. The computer-implemented system of claim 1, wherein in classifying the one or more assets associated with each of the one or more smoke compartments into the plurality of risk levels, the asset classifying subsystem is configured to:
retrieve asset inventory data comprising asset identifiers, asset specifications, installation dates, and maintenance history for each of the one or more assets within each of the one or more smoke compartments from the database;
determine asset criticality levels by analyzing functional importance of each of the one or more assets to life safety operations within the corresponding one or more smoke compartments using predefined criticality matrices;
map asset locations to specific zones within each of the one or more smoke compartments using coordinate data, floor plans, and spatial relationship algorithms to establish the location environment for each asset;
apply the asset class inheritance-based AI models to automatically inherit risk characteristics from parent asset categories and propagate risk classifications to child assets based on hierarchical asset relationships;
execute the asset class acquisition-based AI models to dynamically acquire new risk classifications by analyzing real-time performance data and environmental conditions affecting each of the one or more assets;
correlate the one or more asset classes with the plurality of risk levels associated with each smoke compartment by matching asset safety functions, failure impact potential, and regulatory compliance requirements to the determined plurality of risk levels;
assign weighted risk factors to each of the one or more assets based on proximity to high-risk areas, interdependencies with other critical assets, and potential cascade failure effects within the smoke compartments;
validate asset classifications by cross-referencing determined asset risk levels against regulatory standards, manufacturer specifications, and historical failure patterns for similar assets;
generate asset risk profiles comprising asset class, assigned risk level, location environment factors, and classification confidence scores for each of the one or more assets; and
update asset classifications dynamically in response to changes in smoke compartment risk levels, asset performance degradation, or modifications to the location environment within the smoke compartments.

4. The computer-implemented system of claim 1, wherein in determining the aggregated score of the plurality of risk assessment scores associated with the one or more failed inspection points, the aggregated score determining subsystem is configured to:
identify the one or more failed inspection points by retrieving inspection results data and comparing actual inspection outcomes against required element of performance (EP) standards for each of the one or more inspection points within each of the one or more smoke compartments;
filter the plurality of risk assessment scores to isolate the plurality of risk assessment scores corresponding to the one or more failed inspection points during excluding scores from the one or more inspection points that passed the inspection;
apply one or more aggregation models comprising at least one of: weighted summation, root mean square computations, and maximum value selection, to combine the plurality of risk assessment scores associated with the one or more failed inspection points within each smoke compartment;
incorporate smoke compartment weighting factors based on compartment size, occupancy levels, and critical function designations to adjust the aggregated score computation for each of the one or more smoke compartments;
determine cumulative risk impact by analyzing the combined effect of the plurality of failed inspection points within the same smoke compartment, comprising potential synergistic effects amplifying overall risk levels;
apply temporal decay functions to adjust the plurality of risk assessment scores based on time elapsed as each inspection point failure was identified, wherein recent inspection point failures receive optimized weighting in the aggregated score determination;
normalize the aggregated scores across distinct smoke compartments to adapt consistent comparison and threshold evaluation regardless of compartment size and a number of the one or more assets contained within each compartment;
validate aggregated score accuracy by cross-referencing computed scores against historical incident data and regulatory risk assessment benchmarks for analogous facility types and smoke compartment configurations; and
generate aggregated score breakdown reports documenting the individual risk assessment scores, weighting factors, and computation methodologies used to determine a final aggregated score for each smoke compartment.

5. The computer-implemented system of claim 1, wherein in determining the plurality of ILSMs for each of the one or more assets within each of the one or more smoke compartments, the ILSM determining subsystem is configured to:
compare the aggregated score against the pre-determined threshold value for each of the one or more smoke compartments to identify the one or more smoke compartments requiring the plurality of ILSMs;
retrieve threshold configuration parameters from the database comprising pre-determined threshold values specific to distinct facility types, occupancy classifications, and regulatory requirements applicable to each of the one or more smoke compartments;
identify triggering assets by analyzing which of the one or more assets within each smoke compartment contributed the one or more failed inspection points that caused the aggregated score to exceed the pre-determined threshold value;
correlate the one or more failed inspection points with the plurality of correlating deficiency assets stored in the database to determine relationships between specific deficiencies and applicable ILSM actions;
select corresponding ILSM actions from the plurality of ILSM actions stored in the database based on the type of deficiencies, asset classifications, and smoke compartment characteristics associated with the one or more failed inspection points;
prioritize the plurality of ILSM actions based on severity of risk, regulatory compliance requirements, and potential impact on the one or more individuals within each of the one or more smoke compartments;
validate ILSM appropriateness by cross-referencing selected ILSM actions against regulatory standards, facility policies, and best practices for similar deficiency scenarios;
generate ILSM implementation plans comprising specific actions, required resources, implementation timelines, and responsible parties for each determined ILSM within each affected smoke compartment;
determine ILSM effectiveness metrics to estimate risk reduction achieved by implementing each determined ILSM action based on historical performance data and risk mitigation models; and
generate a report associated with ILSM determinations comprising justification for each selected ILSM action, expected duration of implementation, and criteria for ILSM termination when permanent corrections are completed.

6. The computer-implemented system of claim 1, wherein in configuring the information associated with the selected plurality of ILSM actions with the plurality of IoT controllers for adapting the plurality of IoT controllers to automatically control the one or more assets, the asset controlling subsystem is configured to:
identify IoT controller assignments by mapping each of the plurality of IoT controllers to corresponding one or more assets within each of the one or more smoke compartments based on asset location data and controller communication capabilities;
translate the plurality of ILSM actions into control commands by converting the selected plurality of ILSM actions into machine-readable instructions and control parameters compatible with the plurality of IoT controllers;
establish communication protocols between the asset controlling subsystem and the plurality of IoT controllers using at least one of: wireless communication, wired networks, and mesh networking topologies to enable real-time command transmission;
configure controller operating parameters by programming each IoT controller with specific control logic, safety thresholds, and automated response sequences corresponding to the determined ILSM actions for protecting the one or more individuals;
execute fail-safe mechanisms within each of the plurality of IoT controllers to determine whether safe operation and automatic reversion to safe states when communication failures and system malfunctions occur;
coordinate multi-controller operations by synchronizing actions between the plurality of IoT controllers when ILSM implementation requires coordinated control of interdependent assets across one or more areas of the smoke compartments;
monitor a status of the plurality of IoT controllers by continuously receiving operational feedback, error reports, and performance data from each of the plurality of IoT controllers to verify proper ILSM action execution;
validate control effectiveness by analyzing the real-time sensor data and asset performance metrics to determine that the automatically controlled assets are successfully protecting the one or more individuals as intended by the plurality of ILSM actions;
generate control audit logs documenting a plurality of commands sent to the plurality of IoT controllers, controller responses, and asset control actions performed for regulatory compliance and system troubleshooting purposes; and
update one or more configurations of the plurality of IoT controllers dynamically in response to changes in ILSM requirements, asset status modifications, and emergency conditions that require immediate adjustment of automated control parameters.

7. The computer-implemented system of claim 1, wherein the plurality of subsystems further comprises:
a work order generating subsystem configured to:
generate at least one of one or more maintenance work orders and one or more corrective work orders, based on the determined plurality of ILSMs; and
determine a status of the one or more maintenance work orders that is indicated as not completed and a status of the one or more corrective work orders that comprise one or more reported deficiencies requiring an immediate corrective action;
a profile retrieving subsystem configured to retrieve a risk profile corresponding to the location environment associated with each of the one or more assets in each of the one or more smoke compartments, from the database, wherein the risk profile comprises at least one of the potential risk factors, the plurality of risk levels, plurality of risk assessment scores, and the aggregated score; and
the work order generating subsystem further configured to prioritize each of at least of: the one or more maintenance work orders and the one or more corrective work orders, based on the risk profile.

8. The computer-implemented system of claim 1, wherein the score assigning subsystem is further configured to:
determine at least one of: the one or more failed inspection points correspond to risk assessment assets, the location environment of the one or more assets corresponding to at least one of one or more supplementary facilities and one or more third party vendors, and a proximity to a subsequently discovered risk assessment assets; and
increment the risk assessment score in response to the determined risk assessment assets impacting the aggregated risk assessment score.

9. A computer-implemented method for determining an interim life safety measure (ILSM) to automatically control one or more assets within one or more smoke compartments in a facility, the computer-implemented method comprising:
receiving, by one or more hardware processors associated with a computer-implemented system, real-time sensor data comprising at least one of: smoke density, temperature, humidity, environmental factors and image data captured from the one or more smoke compartments of the facility;
analyzing, by the one or more hardware processors, a plurality of potential risk factors associated with one or more individuals in the one or more smoke compartments of the facility, based on a type of the one or more smoke compartments, wherein analyzing the plurality of potential risk factors, comprises utilizing, by the one or more hardware processors, one or more trained Artificial Intelligence (AI)-based machine learning models, by at least one of:
detecting, by the one or more hardware processors, at least one of: smoke and flames in the one or more smoke compartments, based on large datasets of smoke and non-smoke image data;
analyzing, by the one or more hardware processors, the real-time sensor data from at least one of: temperature and humidity sensors, to predict fire risks in the one or more smoke compartments;
modeling, by the one or more hardware processors, causal relationships between one or more fire risk factors contributing to the fire risks in the one or more smoke compartments;
classifying, by the one or more hardware processors, the sensor data patterns comprising variations in temperature, humidity, and smoke density, indicative of the fire risks in the one or more smoke compartments; and
providing, by the one or more hardware processors, recommendations for at least one of: preventive measures and emergency response based on the sensor data patterns associated with potential fire risks,
wherein the one or more trained AI-based machine learning models comprise at least one of: first convolutional neural networks (CNNs), recurrent neural networks (RNNs), Bayesian networks, first support vector machines (SVMs), and decision trees;
determining, by the one or more hardware processors, a plurality of risk levels for each of the one or more smoke compartments, based on the analyzed plurality of potential risk factors;
in response to determining the plurality of risk levels, classifying, by the one or more hardware processors, one or more assets associated with each of the one or more smoke compartments into the plurality of risk levels, based on at least one of one or more asset classes and a location environment associated with each of the one or more assets in each of the one or more smoke compartments comprising the plurality of risk levels,
wherein the one or more asset classes are generated using at least one of: asset class inheritance-based AI models and asset class acquisition-based AI models, wherein at least one of: the asset class inheritance-based AI models and asset class acquisition-based AI models is trained to:
analyze image data and historical data associated with the one or more assets; and
classify the one or more assets into asset classes based on the analyzed image data and historical data, and
wherein at least one of the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, and Generative Adversarial Networks (GANs);
assigning, by the one or more hardware processors, a plurality of risk assessment scores to each of one or more inspection points corresponding to the classified one or more assets, wherein the risk assessment score corresponds to at least one of an importance and a potential harm created when the one or more inspection point fails an inspection, and wherein each of the one or more inspection points corresponds to a requirement of an element of performance (EP);
determining, by the one or more hardware processors, an aggregated score of the plurality of risk assessment scores associated with one or more failed inspection points for the one or more assets within each of the one or more smoke compartments;
storing, by the one or more hardware processors, information associated with a plurality of Interim Life Safety Measure (ILSM) actions and a plurality of correlating deficiency assets, in a database, wherein each ILSM action defines an action related to protecting the one or more individuals at the facility, and wherein each correlating deficiency asset defines a correlation between a deficiency encountered at the facility and at least one ILSM action;
determining, by the one or more hardware processors, a plurality of ILSMs for each of the one or more assets within each of the one or more smoke compartments, when the aggregated score is greater than a pre-determined threshold value for each of the one or more assets within each of the one or more smoke compartments, wherein the ILSM is a health and safety measure to protect the one or more individuals at the facility,
wherein determining the plurality of ILSMs comprises selecting, by the one or more hardware processors, the plurality of ILSMs from the database, based on deficiencies associated with the one or more failed inspection points and the plurality of correlating deficiency assets;
configuring, by the one or more hardware processors, the information associated with the selected plurality of ILSM actions with a plurality of internet of things (IoT) controllers for adapting the plurality of IoT controllers to automatically control the one or more assets within each of the one or more smoke compartments to protect the one or more individuals in the one or more smoke compartments of the facility, wherein each IoT controller of the plurality of IoT controllers is configured in corresponding one or more assets within each of the one or more smoke compartments; and
providing, by the one or more hardware processors, one or more controlled activities of the one or more assets within each of the one or more smoke compartments, as an output to one or more users through one or more user interfaces associated with one or more electronic devices of the one or more users.

10. The computer-implemented method of claim 9, wherein determining the plurality of risk levels for each of the one or more smoke compartments, based on the analyzed plurality of potential risk factors, comprises:
analyzing, by the one or more hardware processors, one or more correlation patterns between the plurality of potential risk factors and historical fire incident data to determine risk weighting coefficients for each potential risk factor within each of the one or more smoke compartments;
applying, by the one or more hardware processors, the one or more trained AI-based machine learning models comprising at least one of: neural networks, decision trees, and regression models to process the analyzed plurality of potential risk factors and generate quantitative risk assessment values for each of the one or more smoke compartments;
categorizing, by the one or more hardware processors, the quantitative risk assessment values into the plurality of risk levels comprising at least one of: low risk, moderate risk, high risk, and critical risk levels based on predetermined threshold ranges;
incorporating, by the one or more hardware processors, environmental context factors comprising at least one of: occupancy type, building age, construction materials, and facility usage patterns to adjust the plurality of risk levels for each of the one or more smoke compartments;
performing, by the one or more hardware processors, dynamic risk level re-computation in real-time based on changes in the real-time sensor data and updated potential risk factors to maintain current risk level assessments;
validating, by the one or more hardware processors, risk level accuracy by comparing determined risk levels against historical incident patterns and regulatory compliance standards for analogous smoke compartments; and
generating, by the one or more hardware processors, risk level confidence scores indicating the reliability of each determined risk level based on data quality, sensor accuracy, and completeness of the analyzed plurality of potential risk factors.

11. The computer-implemented method of claim 9, wherein classifying the one or more assets associated with each of the one or more smoke compartments into the plurality of risk levels, comprises:
retrieving, by the one or more hardware processors, asset inventory data comprising asset identifiers, asset specifications, installation dates, and maintenance history for each of the one or more assets within each of the one or more smoke compartments from the database;
determining, by the one or more hardware processors, asset criticality levels by analyzing functional importance of each of the one or more assets to life safety operations within the corresponding one or more smoke compartments using predefined criticality matrices;
mapping, by the one or more hardware processors, asset locations to specific zones within each of the one or more smoke compartments using coordinate data, floor plans, and spatial relationship algorithms to establish the location environment for each asset;
applying, by the one or more hardware processors, the asset class inheritance-based AI models to automatically inherit risk characteristics from parent asset categories and propagate risk classifications to child assets based on hierarchical asset relationships;
executing, by the one or more hardware processors, the asset class acquisition-based AI models to dynamically acquire new risk classifications by analyzing real-time performance data and environmental conditions affecting each of the one or more assets;
correlating, by the one or more hardware processors, the one or more asset classes with the plurality of risk levels associated with each smoke compartment by matching asset safety functions, failure impact potential, and regulatory compliance requirements to the determined plurality of risk levels;
assigning, by the one or more hardware processors, weighted risk factors to each of the one or more assets based on proximity to high-risk areas, interdependencies with other critical assets, and potential cascade failure effects within the smoke compartments;
validating, by the one or more hardware processors, asset classifications by cross-referencing determined asset risk levels against regulatory standards, manufacturer specifications, and historical failure patterns for similar assets;
generating, by the one or more hardware processors, asset risk profiles comprising asset class, assigned risk level, location environment factors, and classification confidence scores for each of the one or more assets; and
updating, by the one or more hardware processors, asset classifications dynamically in response to changes in smoke compartment risk levels, asset performance degradation, or modifications to the location environment within the smoke compartments.

12. The computer-implemented method of claim 9, wherein determining the aggregated score of the plurality of risk assessment scores associated with the one or more failed inspection points, comprises:
identifying, by the one or more hardware processors, the one or more failed inspection points by retrieving inspection results data and comparing actual inspection outcomes against required element of performance (EP) standards for each of the one or more inspection points within each of the one or more smoke compartments;
filtering, by the one or more hardware processors, the plurality of risk assessment scores to isolate the plurality of risk assessment scores corresponding to the one or more failed inspection points during excluding scores from the one or more inspection points that passed the inspection;
applying, by the one or more hardware processors, one or more aggregation models comprising at least one of: weighted summation, root mean square computations, and maximum value selection, to combine the plurality of risk assessment scores associated with the one or more failed inspection points within each smoke compartment;
incorporating, by the one or more hardware processors, smoke compartment weighting factors based on compartment size, occupancy levels, and critical function designations to adjust the aggregated score computation for each of the one or more smoke compartments;
determining, by the one or more hardware processors, cumulative risk impact by analyzing the combined effect of the plurality of failed inspection points within the same smoke compartment, comprising potential synergistic effects amplifying overall risk levels;
applying, by the one or more hardware processors, temporal decay functions to adjust the plurality of risk assessment scores based on time elapsed as each inspection point failure was identified, wherein recent inspection point failures receive optimized weighting in the aggregated score determination;
normalizing, by the one or more hardware processors, the aggregated scores across distinct smoke compartments to adapt consistent comparison and threshold evaluation regardless of compartment size and a number of the one or more assets contained within each compartment;
validating, by the one or more hardware processors, aggregated score accuracy by cross-referencing computed scores against historical incident data and regulatory risk assessment benchmarks for analogous facility types and smoke compartment configurations; and
generating, by the one or more hardware processors, aggregated score breakdown reports documenting the individual risk assessment scores, weighting factors, and computation methodologies used to determine a final aggregated score for each smoke compartment.

13. The computer-implemented method of claim 9, wherein determining the plurality of ILSMs for each of the one or more assets within each of the one or more smoke compartments, comprises:
comparing, by the one or more hardware processors, the aggregated score against the pre-determined threshold value for each of the one or more smoke compartments to identify the one or more smoke compartments requiring the plurality of ILSMs;
retrieving, by the one or more hardware processors, threshold configuration parameters from the database comprising pre-determined threshold values specific to distinct facility types, occupancy classifications, and regulatory requirements applicable to each of the one or more smoke compartments;
identifying, by the one or more hardware processors, triggering assets by analyzing which of the one or more assets within each smoke compartment contributed the one or more failed inspection points that caused the aggregated score to exceed the pre-determined threshold value;
correlating, by the one or more hardware processors, the one or more failed inspection points with the plurality of correlating deficiency assets stored in the database to determine relationships between specific deficiencies and applicable ILSM actions;
selecting, by the one or more hardware processors, corresponding ILSM actions from the plurality of ILSM actions stored in the database based on the type of deficiencies, asset classifications, and smoke compartment characteristics associated with the one or more failed inspection points;
prioritizing, by the one or more hardware processors, the plurality of ILSM actions based on severity of risk, regulatory compliance requirements, and potential impact on the one or more individuals within each of the one or more smoke compartments;
validating, by the one or more hardware processors, ILSM appropriateness by cross-referencing selected ILSM actions against regulatory standards, facility policies, and best practices for similar deficiency scenarios;
generating, by the one or more hardware processors, ILSM implementation plans comprising specific actions, required resources, implementation timelines, and responsible parties for each determined ILSM within each affected smoke compartment;
determining, by the one or more hardware processors, ILSM effectiveness metrics to estimate risk reduction achieved by implementing each determined ILSM action based on historical performance data and risk mitigation models; and
generating, by the one or more hardware processors, report associated with ILSM determinations comprising justification for each selected ILSM action, expected duration of implementation, and criteria for ILSM termination when permanent corrections are completed.

14. The computer-implemented method of claim 9, wherein configuring the information associated with the selected plurality of ILSM actions with the plurality of IoT controllers for adapting the plurality of IoT controllers to automatically control the one or more assets, comprises:
identifying, by the one or more hardware processors, IoT controller assignments by mapping each of the plurality of IoT controllers to corresponding one or more assets within each of the one or more smoke compartments based on asset location data and controller communication capabilities;
translating, by the one or more hardware processors, the plurality of ILSM actions into control commands by converting the selected plurality of ILSM actions into machine-readable instructions and control parameters compatible with the plurality of IoT controllers;
establishing, by the one or more hardware processors, communication protocols between the asset controlling subsystem and the plurality of IoT controllers using at least one of: wireless communication, wired networks, and mesh networking topologies to enable real-time command transmission;
configuring, by the one or more hardware processors, controller operating parameters by programming each IoT controller with specific control logic, safety thresholds, and automated response sequences corresponding to the determined ILSM actions for protecting the one or more individuals;
executing, by the one or more hardware processors, fail-safe mechanisms within each of the plurality of IoT controllers to determine whether safe operation and automatic reversion to safe states when communication failures and system malfunctions occur;
coordinating, by the one or more hardware processors, multi-controller operations by synchronizing actions between the plurality of IoT controllers when ILSM implementation requires coordinated control of interdependent assets across one or more areas of the smoke compartments;
monitoring, by the one or more hardware processors, a status of the plurality of IoT controllers by continuously receiving operational feedback, error reports, and performance data from each of the plurality of IoT controllers to verify proper ILSM action execution;
validating, by the one or more hardware processors, control effectiveness by analyzing the real-time sensor data and asset performance metrics to determine that the automatically controlled assets are successfully protecting the one or more individuals as intended by the plurality of ILSM actions;
generating, by the one or more hardware processors, control audit logs documenting a plurality of commands sent to the plurality of IoT controllers, controller responses, and asset control actions performed for regulatory compliance and system troubleshooting purposes; and
updating, by the one or more hardware processors, one or more configurations of the plurality of IoT controllers dynamically in response to changes in ILSM requirements, asset status modifications, and emergency conditions that require immediate adjustment of automated control parameters.

15. The computer-implemented method of claim 9, further comprising:
generating, by the one or more hardware processors, at least one of one or more maintenance work orders and one or more corrective work orders, based on the determined plurality of ILSMs;
determining, by the one or more hardware processors, a status of the one or more maintenance work orders that is indicated as not completed and a status of the one or more corrective work orders that comprise one or more reported deficiencies requiring an immediate corrective action;
retrieving, by the one or more hardware processors, a risk profile corresponding to the location environment associated with each of the one or more assets in each of the one or more smoke compartments, from the database, wherein the risk profile comprises at least one of the potential risk factors, the plurality of risk levels, plurality of risk assessment scores, and the aggregated score; and
prioritizing, by the one or more hardware processors, each of at least of: the one or more maintenance work orders and the one or more corrective work orders, based on the risk profile.

16. The computer-implemented method of claim 9, further comprising:
determining, by the one or more hardware processors, at least one of: the one or more failed inspection points correspond to risk assessment assets, the location environment of the one or more assets corresponding to at least one of one or more supplementary facilities and one or more third party vendors, and a proximity to a subsequently discovered risk assessment assets; and
incrementing, by the one or more hardware processors, the risk assessment score in response to the determined risk assessment assets impacting the aggregated risk assessment score.

17. A non-transitory computer-readable storage medium having instructions stored therein that when executed by one or more hardware processors, cause the one or more hardware processors to execute operations of:
receiving real-time sensor data comprising at least one of: smoke density, temperature, humidity, environmental factors and image data captured from the one or more smoke compartments of a facility;
analyzing a plurality of potential risk factors associated with one or more individuals in the one or more smoke compartments of the facility, based on a type of the one or more smoke compartments, wherein analyzing the plurality of potential risk factors, comprises utilizing, by the one or more hardware processors, one or more trained Artificial Intelligence (AI)-based machine learning models, by at least one of:
detecting at least one of: smoke and flames in the one or more smoke compartments, based on large datasets of smoke and non-smoke image data;
analyzing the real-time sensor data from at least one of: temperature and humidity sensors, to predict fire risks in the one or more smoke compartments;
modeling causal relationships between one or more fire risk factors contributing to the fire risks in the one or more smoke compartments;
classifying the sensor data patterns comprising variations in temperature, humidity, and smoke density, indicative of the fire risks in the one or more smoke compartments; and
providing recommendations for at least one of: preventive measures and emergency response based on sensor data patterns associated with potential fire risks,
wherein the one or more trained AI-based machine learning models comprise at least one of: first convolutional neural networks (CNNs), recurrent neural networks (RNNs), Bayesian networks, first support vector machines (SVMs), and decision trees;
determining a plurality of risk levels for each of the one or more smoke compartments, based on the analyzed plurality of potential risk factors;
in response to determining the plurality of risk levels, classifying, by the one or more hardware processors, one or more assets associated with each of the one or more smoke compartments into the plurality of risk levels, based on at least one of one or more asset classes and a location environment associated with each of the one or more assets in each of the one or more smoke compartments comprising the plurality of risk levels, wherein the one or more asset classes are generated using at least one of: asset class inheritance-based AI models and asset class acquisition-based AI models, wherein the at least one of: the asset class inheritance-based AI models and asset class acquisition-based AI models is trained to:
analyze image data and historical data associated with the one or more assets; and
classify the one or more assets into asset classes based on the analyzed image data and historical data, and
wherein at least one of: the asset class inheritance-based AI models and the asset class acquisition-based AI models comprise at least one of: second Convolutional Neural Networks (CNNs), random forests, Long Short term Memory (LSTM) networks, second Standard Vector Machines (SVMs) based models, and Generative Adversarial Networks (GANs);
assigning a plurality of risk assessment scores to each of one or more inspection points corresponding to the classified one or more assets, wherein the risk assessment score corresponds to at least one of an importance and a potential harm created when the one or more inspection point fails an inspection, and wherein each of the one or more inspection points corresponds to a requirement of an element of performance (EP);
determining an aggregated score of the plurality of risk assessment scores associated with one or more failed inspection points for the one or more assets within each of the one or more smoke compartments;
storing, by the one or more hardware processors, information associated with a plurality of Interim Life Safety Measure (ILSM) actions and a plurality of correlating deficiency assets, in a database, wherein each ILSM action defines an action related to protecting the one or more individuals at the facility, and wherein each correlating deficiency asset defines a correlation between a deficiency encountered at the facility and at least one ILSM action;
determining, by the one or more hardware processors, a plurality of ILSMs for each of the one or more assets within each of the one or more smoke compartments, when the aggregated score is greater than a pre-determined threshold value for each of the one or more assets within each of the one or more smoke compartments, wherein the ILSM is a health and safety measure to protect the one or more individuals at the facility, wherein determining the plurality of ILSMs comprises selecting, by the one or more hardware processors, the plurality of ILSMs from the database, based on deficiencies associated with the one or more failed inspection points and the plurality of correlating deficiency assets;
configuring, by the one or more hardware processors, the information associated with the selected plurality of ILSM actions with a plurality of internet of things (IoT) controllers for adapting the plurality of IoT controllers to automatically control the one or more assets within each of the one or more smoke compartments to protect the one or more individuals in the one or more smoke compartments of the facility, wherein each IoT controller of the plurality of IoT controllers is configured in corresponding one or more assets within each of the one or more smoke compartments; and
providing, by the one or more hardware processors, one or more controlled activities of the one or more assets within each of the one or more smoke compartments, as an output to one or more users through one or more user interfaces associated with one or more electronic devices of the one or more users.

18. The non-transitory computer-readable storage medium of claim 17, wherein determining the plurality of ILSMs for each of the one or more assets within each of the one or more smoke compartments, comprises:
comparing the aggregated score against the pre-determined threshold value for each of the one or more smoke compartments to identify the one or more smoke compartments requiring the plurality of ILSMs;
retrieving threshold configuration parameters from the database comprising pre-determined threshold values specific to distinct facility types, occupancy classifications, and regulatory requirements applicable to each of the one or more smoke compartments;
identifying triggering assets by analyzing which of the one or more assets within each smoke compartment contributed the one or more failed inspection points that caused the aggregated score to exceed the pre-determined threshold value;
correlating the one or more failed inspection points with the plurality of correlating deficiency assets stored in the database to determine relationships between specific deficiencies and applicable ILSM actions;
selecting corresponding ILSM actions from the plurality of ILSM actions stored in the database based on the type of deficiencies, asset classifications, and smoke compartment characteristics associated with the one or more failed inspection points;
prioritizing the plurality of ILSM actions based on severity of risk, regulatory compliance requirements, and potential impact on the one or more individuals within each of the one or more smoke compartments;
validating ILSM appropriateness by cross-referencing selected ILSM actions against regulatory standards, facility policies, and best practices for similar deficiency scenarios;
generating ILSM implementation plans comprising specific actions, required resources, implementation timelines, and responsible parties for each determined ILSM within each affected smoke compartment;
determining ILSM effectiveness metrics to estimate risk reduction achieved by implementing each determined ILSM action based on historical performance data and risk mitigation models; and
generating report associated with ILSM determinations comprising justification for each selected ILSM action, expected duration of implementation, and criteria for ILSM termination when permanent corrections are completed.

19. The non-transitory computer-readable storage medium of claim 17, wherein configuring the information associated with the selected plurality of ILSM actions with the plurality of IoT controllers for adapting the plurality of IoT controllers to automatically control the one or more assets, comprises:
identifying IoT controller assignments by mapping each of the plurality of IoT controllers to corresponding one or more assets within each of the one or more smoke compartments based on asset location data and controller communication capabilities;
translating the plurality of ILSM actions into control commands by converting the selected plurality of ILSM actions into machine-readable instructions and control parameters compatible with the plurality of IoT controllers;
establishing communication protocols between the asset controlling subsystem and the plurality of IoT controllers using at least one of: wireless communication, wired networks, and mesh networking topologies to enable real-time command transmission;
configuring controller operating parameters by programming each IoT controller with specific control logic, safety thresholds, and automated response sequences corresponding to the determined ILSM actions for protecting the one or more individuals;
executing fail-safe mechanisms within each of the plurality of IoT controllers to determine whether safe operation and automatic reversion to safe states when communication failures and system malfunctions occur;
coordinating multi-controller operations by synchronizing actions between the plurality of IoT controllers when ILSM implementation requires coordinated control of interdependent assets across one or more areas of the smoke compartments;
monitoring a status of the plurality of IoT controllers by continuously receiving operational feedback, error reports, and performance data from each of the plurality of IoT controllers to verify proper ILSM action execution;
validating control effectiveness by analyzing the real-time sensor data and asset performance metrics to determine that the automatically controlled assets are successfully protecting the one or more individuals as intended by the plurality of ILSM actions;
generating control audit logs documenting a plurality of commands sent to the plurality of IoT controllers, controller responses, and asset control actions performed for regulatory compliance and system troubleshooting purposes; and
updating one or more configurations of the plurality of IoT controllers dynamically in response to changes in ILSM requirements, asset status modifications, and emergency conditions that require immediate adjustment of automated control parameters.

20. The non-transitory computer-readable storage medium of claim 17, further comprising:
determining at least one of: the one or more failed inspection points correspond to risk assessment assets, the location environment of the one or more assets corresponding to at least one of one or more supplementary facilities and one or more third party vendors, and a proximity to a subsequently discovered risk assessment assets; and
incrementing the risk assessment score in response to the determined risk assessment assets impacting the aggregated risk assessment score.
